(19) United States
(12) Patent Application Publication
(10) Pub. No.: US 2006/0241666 A1
(43) Pub. Date: Oct. 26, 2006

(54) Method and apparatus for body fluid sampling and analyte sensing

(76) Inventors: Barry Dean Briggs, Campbell, CA (US); Tom Schulte, Palo Alto, CA (US); Don Alden, Sunnyvale, CA (US); James Ross, Livermore, CA (US); Ganapati Mauze, Sunnyvale, CA (US); Dominique M. Freeman, La Honda, CA (US)

Correspondence Address: HELLER EHRMAN LLP, 275 MIDDLEFIELD ROAD, MENLO PARK, CA, 94025-3506, US

(21) Appl. No.: 10/560,263

(22) PCT Filed: Jun. 14, 2004
(86) PCT No.: PCT/US04/19125

§ 371 Date: Jun. 23, 2006

Related U.S. Application Data
(60) Provisional application No. 60/478,041, filed on Jun. 11, 2003.
(60) Provisional application No. 60/478,662, filed on Jun. 13, 2003.
(60) Provisional application No. 60/478,692, filed on Jun. 13, 2003.
(60) Provisional application No. 60/483,324, filed on Jun. 27, 2003.

(51) Int. Cl. A61B 17/32

(52) U.S. Cl. 606/181

(57) ABSTRACT

The method comprises providing a lancing device comprising a penetrating member (68) driver having a position sensor (74) and a processor (60) that can determine the relative position and velocity of the penetrating member (18, 72) based on measuring relative position of the penetrating member with respect to time; providing a predetermined velocity control trajectory based on a model of the driver (68) and a model of tissue to be contacted. Furthermore, a feedforward control is able to maintain penetrating member velocity along the trajectory.

### BACKGROUND OF THE INVENTION

Lancing devices are known in the medical health-care products industry for piercing the skin to produce blood for analysis. Typically, a drop of blood for this type of analysis is obtained by making a small incision in the fingertip, creating a small wound, which generates a small blood droplet on the surface of the skin.

Early methods of lancing included piercing or slicing the skin with a needle or razor. Current methods utilize lancing devices that contain a multitude of spring, cam and mass actuators to drive the lancet. These include cantilever springs, diaphragms, coil springs, as well as gravity plumbs used to drive the lancet. The device may be held against the skin and mechanically triggered to ballistically launch the lancet. Unfortunately, the pain associated with each lancing event using known technology discourages patients from testing. In addition to vibratory stimulation of the skin as the driver impacts the end of a launcher stop, known spring based devices have the possibility of firing lancets that harmonically oscillate against the patient tissue, causing multiple strikes due to recoil. This recoil and multiple strikes of the lancet is one major impediment to patient compliance with a structured glucose monitoring regime.

Another impediment to patient compliance is the lack of spontaneous blood flow generated by known lancing technology. In addition to the pain as discussed above, a patient may need more than one lancing event to obtain a blood sample since spontaneous blood generation is unreliable using known lancing technology. Thus the pain is multiplied by the number of attempts required by a patient to successfully generate spontaneous blood flow. Different skin thickness may yield different results in terms of pain perception, blood yield and success rate of obtaining blood between different users of the lancing device. Known devices poorly account for these skin thickness variations.

Variations in skin thickness including the stratum corneum and hydration of the epidermis can yield different results between different users. Spontaneous blood droplet generation is dependent on reaching the blood capillaries and venuoles, which yield the blood sample. It is therefore an issue of correct depth of penetration of the cutting device. Due to variations in skin thickness and hydration, some types of skin will deform more before cutting starts, and hence the actual depth of penetration will be less, resulting in less capillaries and venuoles cut and less spontaneous blood generation.

Known lancing devices fail to provide accurate sensing of lancet position. Thus they do not know exactly how far the penetrating member has cut into the tissue. This lack of position sensing is one reason for more painful lancing associated with known fluid sampling devices.

Additionally, known lancing devices fail to have sufficiently accurate control of lancet position and velocity to achieve a spontaneous blood generation in a relatively pain free manner.

### SUMMARY OF THE INVENTION

The present invention provides solutions for at least some of the drawbacks discussed above. The technical field relates to the lancing of the finger to obtain a body fluid or blood sample for the analysis of that sample. Because the penetration distance is a strong predictor of the success of the lancing event for spontaneous blood generation, the ability of the device to accurately control this distance is of interest. Specifically, some embodiments of the present invention provide an improved body fluid sampling device. For some embodiments of penetrating member drivers, the invention provides improved methods for controlling the velocity and cutting efficient of a penetrating member. At least some of these and other objectives described herein will be met by embodiments of the present invention.

In one aspect, the present invention provides improved lancing devices operating with adaptive control algorithms. Because of the very high speeds that embodiments of the present invention may move their penetrating members, feedback control may not be sufficient, due to the short amount of time available. In one embodiment, the present invention provides desired parameters, based on the models of the penetrating member, the penetrating member driver, and the targeted tissue. Based on this model, the system may have predictive information stored in lookup tables on how to drive the penetrating member driver and when to apply braking force so that the device performs as desired to arrive at a desired depth and to provide a desired level of cutting efficiency and/or performance.

In one embodiment, a method of controlling a penetrating member is provided. The method comprises providing a lancing device having a penetrating member driver with a position sensor and a processor that can determine the relative position and velocity of the penetrating member based on measuring relative position of the penetrating member with respect to time; providing a look up table having desired velocity trajectory based on empirical data; and using control to adjust lancet velocity to maintain penetrating member velocity along said trajectory.

In another embodiment, the present invention relates to the way that an electronically driven lancing device controls the trajectory of the inbound lancet up to the point of maximum extension or penetration into a target tissue. This is the point of maximum penetration of the lancet into the skin. This embodiment of the present invention comprises a control algorithm, that when combined with the necessary hardware to execute the control instructions, increases the depth accuracy of the penetrating member. The present invention also provides improved cutting efficiency by providing lancet behavior that is optimized for cutting tissue.

In one aspect, the present invention involves learning through testing what the ideal setup parameters are and then using more complicated feedback systems to get results similar to a feed-forward system.

In other aspects, the present invention may involve manual braking, braking with zero residual energy, braking only, preserving acceleration, and appropriate force for smart braking.

The system may further comprise means for coupling the force generator with one of the penetrating members.

The system may further comprise a penetrating member sensor positioned to monitor a penetrating member coupled to the force generator, the penetrating member sensor configured to provide information relative to a depth of penetration of a penetrating member through a skin surface.

The depth of penetration may be about 100 to 2500 microns.

The depth of penetration may be about 500 to 750 microns.

The depth of penetration may be, in this nonlimiting example, no more than about 1000 microns beyond a stratum corneum thickness of a skin surface.

The depth of penetration may be no more than about 500 microns beyond a stratum corneum thickness of a skin surface.

The depth of penetration may be no more than about 300 microns beyond a stratum corneum thickness of a skin surface.

The depth of penetration may be less than a sum of a stratum corneum thickness of a skin surface and 400 microns.

The penetrating member sensor may be further configured to control velocity of a penetrating member.

The active penetrating member may move along a substantially linear path into the tissue.

The active penetrating member may move along an at least partially curved path into the tissue.

The driver may be a voice coil drive force generator.

The driver may be a rotary voice coil drive force generator.

The penetrating member sensor may be coupled to a processor with control instructions for the penetrating member driver.

The processor may include a memory for storage and retrieval of a set of penetrating member profiles utilized with the penetrating member driver.

The processor may be utilized to monitor position and speed of a penetrating member as the penetrating member moves in a first direction.

The processor may be utilized to adjust an application of force to a penetrating member to achieve a desired speed of the penetrating member.

The processor may be utilized to adjust an application of force to a penetrating member when the penetrating member contacts a target tissue so that the penetrating member penetrates the target tissue within a desired range of speed.

The processor may be utilized to monitor position and speed of a penetrating member as the penetrating member moves in the first direction toward a target tissue, wherein the application of a launching force to the penetrating member is controlled based on position and speed of the penetrating member.

The processor may be utilized to control a withdraw force to the penetrating member so that the penetrating member moves in a second direction away from the target tissue.

In the first direction, the penetrating member may move toward the target tissue at a speed that is different than a speed at which the penetrating member moves away from the target tissue.

In the first direction the penetrating member may move toward the target tissue at a speed that is greater than a speed at which the penetrating member moves away from the target tissue.

The speed of a penetrating member in the first direction may be the range of about 2.0 to 10.0 m/sec.

The average velocity of the penetrating member during a tissue penetration stroke in the first direction may be about 100 to about 1000 times greater than the average velocity of the penetrating member during a withdrawal stroke in a second direction.

A further understanding of the nature and advantages of the invention will become apparent by reference to the remaining portions of the specification and drawings.

### BRIEF DESCRIPTION OF THE DRAWINGS

FIG. 1 illustrates an embodiment of a controllable force driver in the form of a cylindrical electric penetrating member driver using a coiled solenoid-type configuration.

FIG. 2A illustrates a displacement over time profile of a penetrating member driven by a harmonic spring/mass system.

FIG. 2B illustrates the velocity over time profile of a penetrating member driver by a harmonic spring/mass system.

FIG. 2C illustrates a displacement over time profile of an embodiment of a controllable force driver.

FIG. 2D illustrates a velocity over time profile of an embodiment of a controllable force driver.

FIG. 3 is a diagrammatic view illustrating a controlled feed-back loop.

FIG. 4 is a perspective view of a tissue penetration device having features of the invention.

FIG. 5 is an elevation view in partial longitudinal section of the tissue penetration device of FIG. 4.

FIG. 6 shows a cutaway perspective view of one embodiment of the device according to the present invention.

FIG. 7 shows an exploded perspective view of one embodiment of the present invention.

FIGS. 8 and 9 show top and bottom views of one embodiment of a chassis for use with the present invention.

FIGS. 10 to 14 show various views of a cartridge according to the present invention.

FIG. 15 shows one embodiment of cartridge with one embodiment of a seal and an analyte detecting member layer.

FIG. 16 shows one embodiment of the analyte detecting member layer.

FIGS. 17-21 show still further embodiments of a cartridge according to the present invention.

FIG. 22-29 show other embodiments of housing multiple penetrating members.

FIGS. 30 and 31 show a simplified launcher for use with a cartridge.

FIGS. 32-37 show still further embodiments of a cartridge according to the present invention.

FIGS. 38-39 show embodiments of a clutch for engaging a penetrating member.

FIGS. 40-42 show a device for loading a plurality of penetrating members.

FIGS. 43-52 show embodiments for engaging penetrating members.

FIGS. 53-58 show various configurations for housing a plurality of penetrating members.

FIGS. 59-82 show still further embodiments for housing a plurality of penetrating members.

FIGS. 83-87 show various perspective views of one embodiment of a device for breaking a seal and rotating the cartridge.

FIGS. 88 to 91 show views of a cam and cam follower for controlling motion of a punch device.

FIGS. 92-93 show still further embodiments of a device according to the present invention.

FIGS. 94-99 show perspective views of a housing according to the present invention.

FIGS. 100-101 show a schematic of a device according to the present invention.—FIGS. 102-103 show perspective views of a housing according to the present invention.

FIG. 104 shows a schematic of a device according to the present invention.—

FIG. 105 shows a close-up view of a cartridge according to the present invention.

FIG. 106 shows a close up of a punch according to the present invention.

FIG. 107 shows one embodiment of disc for use with the present invention.

FIG. 108 shows one view of the disc in a penetrating member device.

FIG. 109 shows another embodiment of a device that may use a disc as described in FIG. 107.

FIG. 110 shows a cutaway perspective view of a device housing a cartridge according to the present invention.

FIGS. 111-113 shows perspective views of interior elements for indexing a cartridge according to the present invention.

FIG. 114 shows elements for indexing a cartridge.

### DESCRIPTION OF THE SPECIFIC EMBODIMENTS

The present invention provides a multiple analyte detecting member solution for body fluid sampling. Specifically, some embodiments of the present invention provides a multiple analyte detecting member and multiple penetrating member solution to measuring analyte levels in the body. The invention may use a high density design. It may use penetrating members of smaller size, such as but not limited to diameter or length, than known lancets. The device may be used for multiple lancing events without having to remove a disposable from the device. The invention may provide improved sensing capabilities. At least some of these and other objectives described herein will be met by embodiments of the present invention.

It is to be understood that both the foregoing general description and the following detailed description are exemplary and explanatory only and are not restrictive of the invention, as claimed. It must be noted that, as used in the specification and the appended claims, the singular forms “a”, “an” and “the” include plural referents unless the context clearly dictates otherwise. Thus, for example, reference to “a material” may include mixtures of materials, reference to “a chamber” may include multiple chambers, and the like. References cited herein are hereby incorporated by reference in their entirety, except to the extent that they conflict with teachings explicitly set forth in this specification.

In this specification and in the claims which follow, reference will be made to a number of terms which shall be defined to have the following meanings:

“Optional” or “optionally” means that the subsequently described circumstance may or may not occur, so that the description includes instances where the circumstance occurs and instances where it does not. For example, if a device optionally contains a feature for analyzing a blood sample, this means that the analysis feature may or may not be present, and, thus, the description includes structure wherein a device possesses the analysis feature and structures wherein the analysis feature is not present.

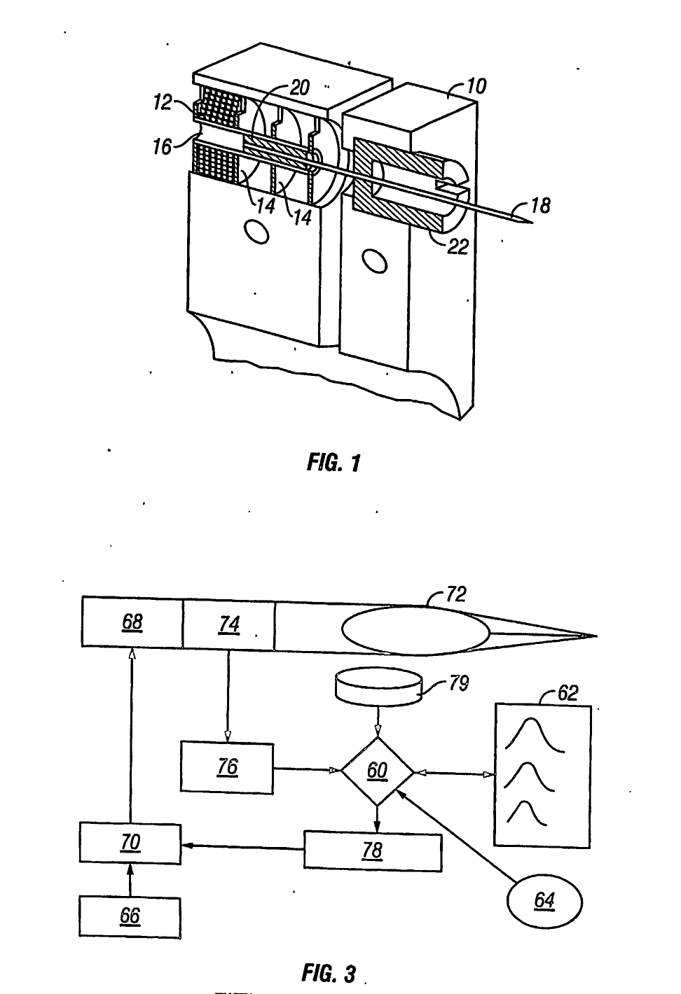

The present invention may be used with a variety of different penetrating member drivers. It is contemplated that these penetrating member drivers may be spring based, solenoid based, magnetic driver based, nanomuscle based, or based on any other mechanism useful in moving a penetrating member along a path into tissue. It should be noted that the present invention is not limited by the type of driver used with the penetrating member feed mechanism. One suitable penetrating member driver for use with the present invention is shown in FIG. 1. This is an embodiment of a solenoid type electromagnetic driver that is capable of driving an iron core or slug mounted to the penetrating member assembly using a direct current (DC) power supply. The electromagnetic driver includes a driver coil pack that is divided into three separate coils along the path of the penetrating member, two end coils and a middle coil. Direct current is alternated to the coils to advance and retract the penetrating member. Although the driver coil pack is shown with three coils, any suitable number of coils may be used, for example, 4, 5, 6, 7 or more coils may be used.

The present invention may be used with a variety of different penetrating member drivers. It is contemplated that these penetrating member drivers may be spring based, solenoid based, magnetic driver based, nanomuscle based, or based on any other mechanism useful in moving a penetrating member along a path into tissue. It should be noted that the present invention is not limited by the type of driver used with the penetrating member feed mechanism. One suitable penetrating member driver for use with the present invention is shown in FIG. 1. This is an embodiment of a solenoid type electromagnetic driver that is capable of driving an iron core or slug mounted to the penetrating member assembly using a direct current (DC) power supply. The electromagnetic driver includes a driver coil pack that is divided into three separate coils along the path of the penetrating member, two end coils and a middle coil. Direct current is alternated to the coils to advance and retract the penetrating member. Although the driver coil pack is shown with three coils, any suitable number of coils may be used, for example, 4, 5, 6, 7 or more coils may be used.

Referring to the embodiment of FIG. 1, the stationary iron housing 10 may contain the driver coil pack with a first coil 12 flanked by iron spacers 14 which concentrate the magnetic flux at the inner diameter creating magnetic poles. The inner insulating housing 16 isolates the penetrating member 18 and iron core 20 from the coils and provides a smooth, low friction guide surface. The penetrating member guide 22 further centers the penetrating member 18 and iron core 20. The penetrating member 18 is protracted and retracted by alternating the current between the first coil 12, the middle coil, and the third coil to attract the iron core 20. Reversing the coil sequence and attracting the core and penetrating member back into the housing retracts the penetrating member. The penetrating member guide 22 also serves as a stop for the iron core 20 mounted to the penetrating member 18.

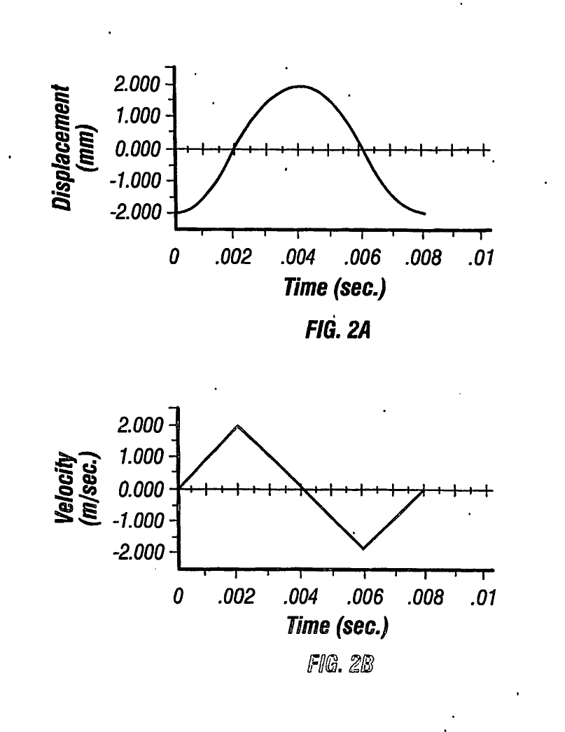

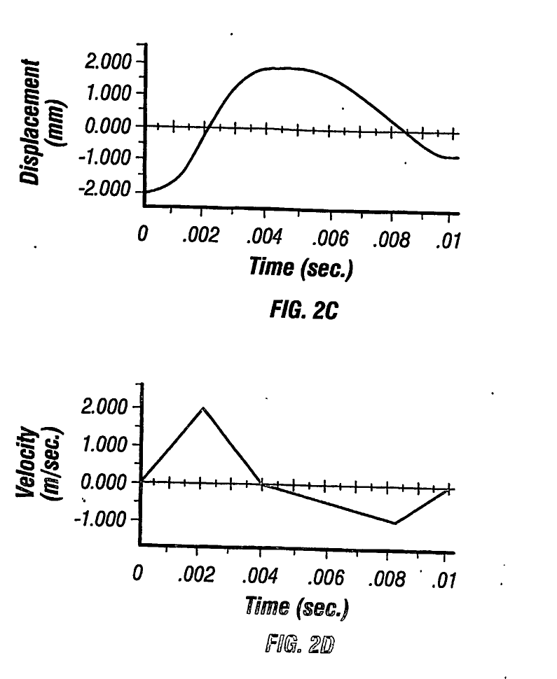

As discussed above, tissue penetration devices which employ spring or cam driving methods have a symmetrical or nearly symmetrical actuation displacement and velocity profiles on the advancement and retraction of the penetrating member as shown in FIGS. 2 and 3. In most of the available lancet devices, once the launch is initiated, the stored energy determines the velocity profile until the energy is dissipated. Controlling impact, retraction velocity, and dwell time of the penetrating member within the tissue can be useful in order to achieve a high success rate while accommodating variations in skin properties and minimize pain. Advantages can be achieved by taking into account of the fact that tissue dwell time is related to the amount of skin deformation as the penetrating member tries to puncture the surface of the skin and variance in skin deformation from patient to patient based on skin hydration.

In this embodiment, the ability to control velocity and depth of penetration may be achieved by use of a controllable force driver where feedback is an integral part of driver control. Such drivers can control either metal or polymeric penetrating members or any other type of tissue penetration element. The dynamic control of such a driver is illustrated in FIG. 2C which illustrates an embodiment of a controlled displacement profile and FIG. 2D which illustrates an embodiment of a the controlled velocity profile. These are compared to FIGS. 2A and 2B, which illustrate embodiments of displacement and velocity profiles, respectively, of a harmonic spring/mass powered driver. Reduced pain can be achieved by using impact velocities of greater than about 2 m/s entry of a tissue penetrating element, such as a lancet, into tissue. Other suitable embodiments of the penetrating member driver are described in commonly assigned, copending U.S. patent application Ser. No. 10/127,395, (Attorney Docket No. 38187-2551) filed Apr. 19, 2002 and previously incorporated herein.

FIG. 3 illustrates the operation of a feedback loop using a processor 60. The processor 60 stores profiles 62 in non-volatile memory. A user inputs information 64 about the desired circumstances or parameters for a lancing event. The processor 60 selects a driver profile 62 from a set of alternative driver profiles that have been preprogrammed in the processor 60 based on typical or desired tissue penetration device performance determined through testing at the factory or as programmed in by the operator. The processor 60 may customize by either scaling or modifying the profile based on additional user input information 64. Once the processor has chosen and customized the profile, the processor 60 is ready to modulate the power from the power supply 66 to the penetrating member driver 68 through an amplifier 70. The processor 60 may measure the location of the penetrating member 72 using a position sensing mechanism 74 through an analog to digital converter 76 linear encoder or other such transducer. Examples of position sensing mechanisms have been described in the embodiments above and may be found in the specification for commonly assigned, copending U.S. patent application Ser. No. 10/127,395, (Attorney Docket No. 38187-2551) filed Apr. 19, 2002 and previously incorporated herein. The processor 60 calculates the movement of the penetrating member by comparing the actual profile of the penetrating member to the predetermined profile. The processor 60 modulates the power to the penetrating member driver 68 through a signal generator 78, which may control the amplifier 70 so that the actual velocity profile of the penetrating member does not exceed the predetermined profile by more than a preset error limit. The error limit is the accuracy in the control of the penetrating member.

After the lancing event, the processor 60 can allow the user to rank the results of the lancing event. The processor 60 stores these results and constructs a database 80 for the individual user. Using the database 79, the processor 60 calculates the profile traits such as degree of painlessness, success rate, and blood volume for various profiles 62 depending on user input information 64 to optimize the profile to the individual user for subsequent lancing cycles. These profile traits depend on the characteristic phases of penetrating member advancement and retraction. The processor 60 uses these calculations to optimize profiles 62 for each user. In addition to user input information 64, an internal clock allows storage in the database 79 of information such as the time of day to generate a time stamp for the lancing event and the time between lancing events to anticipate the user's diurnal needs. The database stores information and statistics for each user and each profile that particular user uses.

In addition to varying the profiles, the processor 60 can be used to calculate the appropriate penetrating member diameter and geometry suitable to realize the blood volume required by the user. For example, if the user requires about 1-5 microliter volume of blood, the processor 60 may select a 200 micron diameter penetrating member to achieve these results. For each class of lancet, both diameter and lancet tip geometry, is stored in the processor 60 to correspond with upper and lower limits of attainable blood volume based on the predetermined displacement and velocity profiles.

The lancing device is capable of prompting the user for information at the beginning and the end of the lancing event to more adequately suit the user. The goal is to either change to a different profile or modify an existing profile. Once the profile is set, the force driving the penetrating member is varied during advancement and retraction to follow the profile. The method of lancing using the lancing device comprises selecting a profile, lancing according to the selected profile, determining lancing profile traits for each characteristic phase of the lancing cycle, and optimizing profile traits for subsequent lancing events.

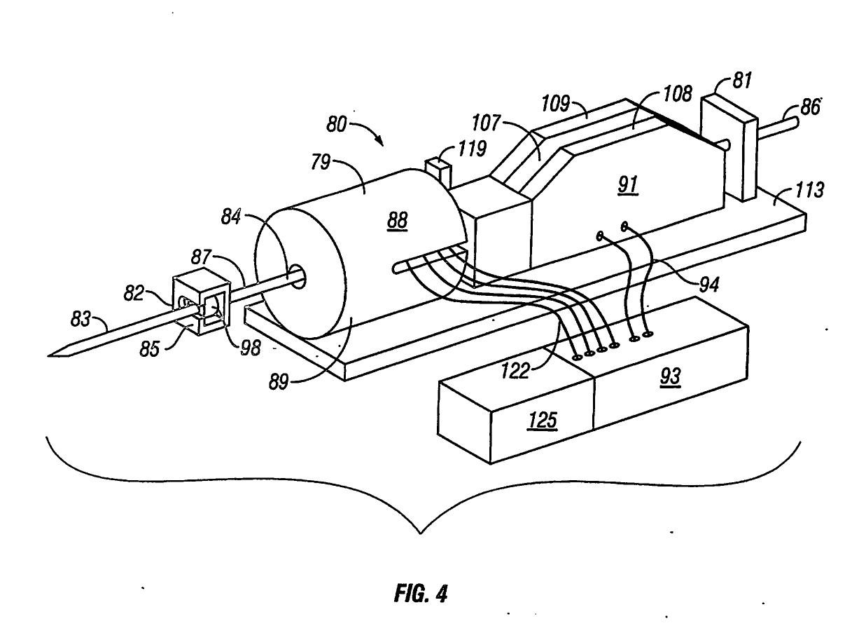

FIG. 4 illustrates an embodiment of a tissue penetration device, more specifically, a lancing device 80 that includes a controllable driver 179 coupled to a tissue penetration element. The lancing device 80 has a proximal end 81 and a distal end 82. At the distal end 82 is the tissue penetration element in the form of a penetrating member 83, which is coupled to an elongate coupler shaft 84 by a drive coupler 85. The elongate coupler shaft 84 has a proximal end 86 and a distal end 87. A driver coil pack 88 is disposed about the elongate coupler shaft 84 proximal of the penetrating member 83. A position sensor 91 is disposed about a proximal portion 92 of the elongate coupler shaft 84 and an electrical conductor 94 electrically couples a processor 93 to the position sensor 91. The elongate coupler shaft 84 driven by the driver coil pack 88 controlled by the position sensor 91 and processor 93 form the controllable driver, specifically, a controllable electromagnetic driver.

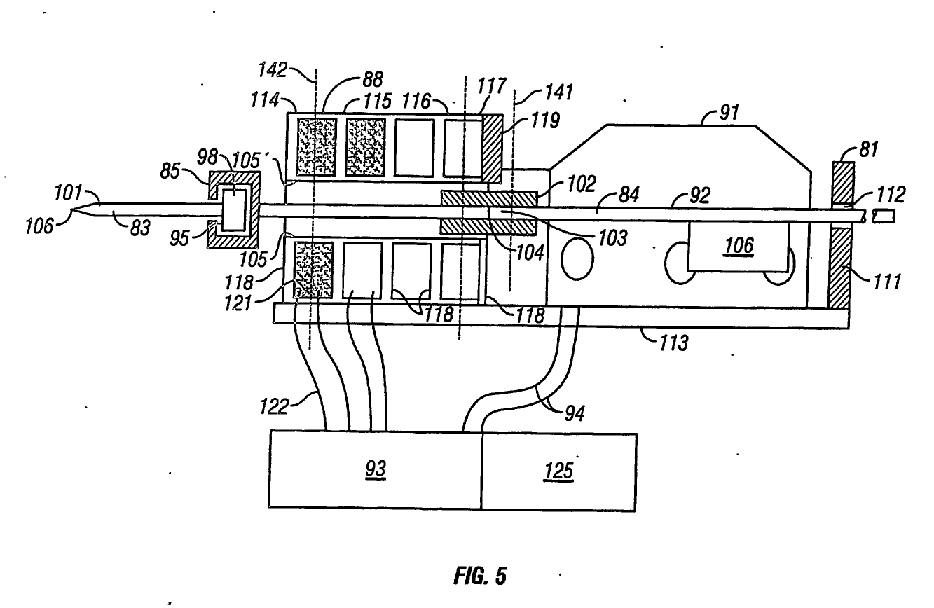

Referring to FIG. 5, the lancing device 80 can be seen in more detail, in partial longitudinal section. The penetrating member 83 has a proximal end 95 and a distal end 96 with a sharpened point at the distal end 96 of the penetrating member 83 and a drive head 98 disposed at the proximal end 95 of the penetrating member 83. A penetrating member shaft 201 is disposed between the drive head 98 and the sharpened point 97. The penetrating member shaft 201 may be comprised of stainless steel, or any other suitable material or alloy and have a transverse dimension of about 0.1 to about 0.4 mm. The penetrating member shaft may have a length of about 3 mm to about 50 mm, specifically, about 15 mm to about 20 mm. The drive head 98 of the penetrating member 83 is an enlarged portion having a transverse dimension greater than a transverse dimension of the penetrating member shaft 201 distal of the drive head 98. This configuration allows the drive head 98 to be mechanically captured by the drive coupler 85. The drive head 98 may have a transverse dimension of about 0.5 to about 2 mm.

A magnetic member 102 is secured to the elongate coupler shaft 84 proximal of the drive coupler 85 on a distal portion 203 of the elongate coupler shaft 84. The magnetic member 102 is a substantially cylindrical piece of magnetic material having an axial lumen 204 extending the length of the magnetic member 102. The magnetic member 102 has an outer transverse dimension that allows the magnetic member 102 to slide easily within an axial lumen 105 of a low friction, possibly lubricious, polymer guide tube 105′ disposed within the driver coil pack 88. The magnetic member 102 may have an outer transverse dimension of about 1.0 to about 5.0 mm, specifically, about 2.3 to about 2.5 mm. The magnetic member 102 may have a length of about 3.0 to about 5.0 mm, specifically, about 4.7 to about 4.9 mm. The magnetic member 102 can be made from a variety of magnetic materials including ferrous metals such as ferrous steel, iron, ferrite, or the like. The magnetic member 102 may be secured to the distal portion 203 of the elongate coupler shaft 84 by a variety of methods including adhesive or epoxy bonding, welding, crimping or any other suitable method.

Proximal of the magnetic member 102, an optical encoder flag 206 is secured to the elongate coupler shaft 84. The optical encoder flag 206 is configured to move within a slot 107 in the position sensor 91. The slot 107 of the position sensor 91 is formed between a first body portion 108 and a second body portion 109 of the position sensor 91. The slot 107 may have separation width of about 1.5 to about 2.0 mm. The optical encoder flag 206 can have a length of about 14 to about 18 mm, a width of about 3 to about 5 mm and a thickness of about 0.04 to about 0.06 mm.

The optical encoder flag 206 interacts with various optical beams generated by LEDs disposed on or in the position sensor body portions 108 and 109 in a predetermined manner. The interaction of the optical beams generated by the LEDs of the position sensor 91 generates a signal that indicates the longitudinal position of the optical flag 206 relative to the position sensor 91 with a substantially high degree of resolution. The resolution of the position sensor 91 may be about 200 to about 400 cycles per inch, specifically, about 350 to about 370 cycles per inch. The position sensor 91 may have a speed response time (position/time resolution) of 0 to about 120,000 Hz, where one dark and light stripe of the flag constitutes one Hertz, or cycle per second. The position of the optical encoder flag 206 relative to the magnetic member 102, driver coil pack 88 and position sensor 91 is such that the optical encoder 91 can provide precise positional information about the penetrating member 83 over the entire length of the penetrating member's power stroke.

An optical encoder that is suitable for the position sensor 91 is a linear optical incremental encoder, model HEDS 9200, manufactured by Agilent Technologies. The model HEDS 9200 may have a length of about 20 to about 30 mm, a width of about 8 to about 12 mm, and a height of about 9 to about 11 mm. Although the position sensor 91 illustrated is a linear optical incremental encoder, other suitable position sensor embodiments could be used, provided they posses the requisite positional resolution and time response. The HEDS 9200 is a two channel device where the channels are 90 degrees out of phase with each other. This results in a resolution of four times the basic cycle of the flag. These quadrature outputs make it possible for the processor to determine the direction of penetrating member travel. Other suitable position sensors include capacitive encoders, analog reflective sensors, such as the reflective position sensor discussed above, and the like. %

A coupler shaft guide 111 is disposed towards the proximal end 81 of the lancing device 80. The guide 111 has a guide lumen 112 disposed in the guide 111 to slidingly accept the proximal portion 92 of the elongate coupler shaft 84. The guide 111 keeps the elongate coupler shaft 84 centered horizontally and vertically in the slot 102 of the optical encoder 91.

In another aspect of the present invention, a still further embodiment of a body fluid sampling device will now be described. Additional details of a suitable body fluid sampling device can be found in commonly assigned, copending U.S. patent application Ser. No. ______ (Attorney Docket No. 38187-2662) filed May 1, 2003, fully incorporated by reference for all purposes.

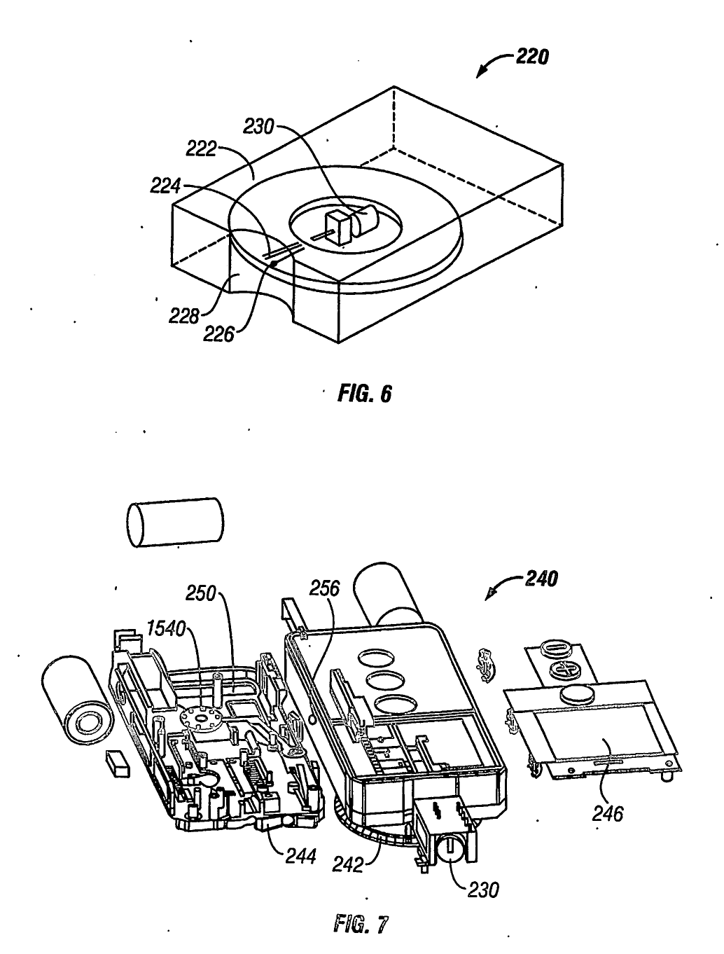

Referring now to FIG. 6, one embodiment of a sampling device 220 is shown. In this embodiment, a cartridge 222 have a disc shape may be used to house a plurality fo penetrating members 224 that may extend outward from an opening 226. A finger or other tissue may be placed at interface 228. It should be understood that a variety of different penetrating members may be used including but not limited to solid, elongate members or patent needle members. In this embodiment, a penetrating member driver 230 may be used to individually engage each penetrating member 224. The penetrating member driver 230 may include those described herein those described in commonly assigned, copending U.S. patent application Ser. No. 10/127,395 (Attorney Docket No. 38187-2551) filed Apr. 19, 2002 or U.S. patent application Ser. No. 10/425,815 (Attorney Docket No. 38187-2663) filed May 30, 2003, each fully incorporated by reference for all purposes

Referring now to FIG. 7, a still further embodiment of a fluid sampling device 240 is shown. In this embodiment, a cartridge 242 is shown which may be inserted into the underside of the device 240. A front end 244 is shown. In this embodiment a solenoid may be used as the penetrating member driver. It should be understood that FIG. 7 shows an exploded view of the components used in the present embodiment. A visual display 246 may be used for various reasons including but not limited to relaying information to the user, to display lancing performance, to provide device status, and other information as described in commonly assigned, copending U.S. patent application Ser. No. 10/237,261 (Attorney Docket No. 38187-2595) filed Sep. 5, 2002 and U.S. patent application Ser. No. 10/335,215 (Attorney Docket No. 38187-2634), fully incorporated herein by reference.

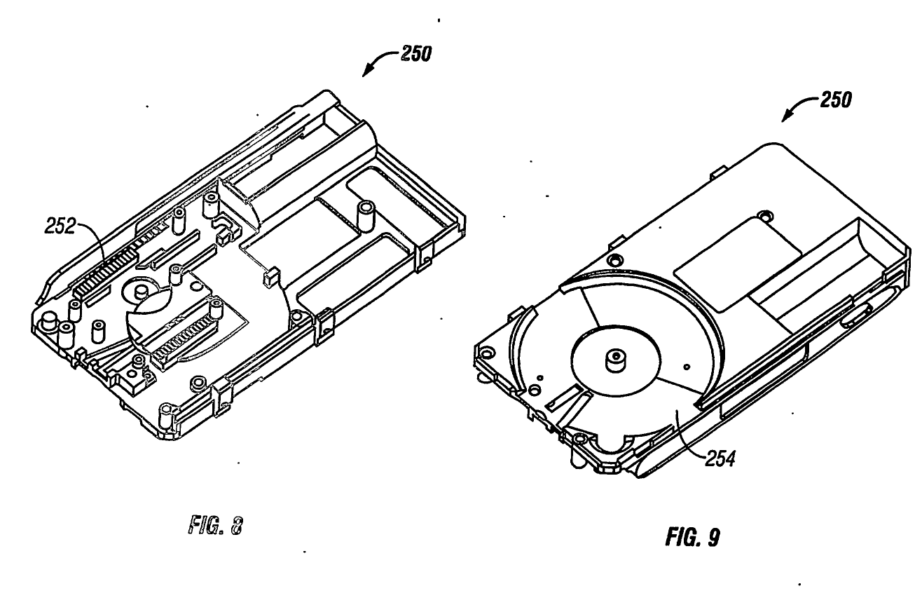

Referring now to FIGS. 8 and 9, top and bottom view of a chassis 250 for use with the device 240 is shown in further detail. FIG. 8 shows various toothed surfaces 252. FIG. 9 shows an underside with a recessed portion 254 for receiving a cartridge 242. The cartridge 242 may be rotated or indexed based on movement of the slider 256 (see FIG. 7).

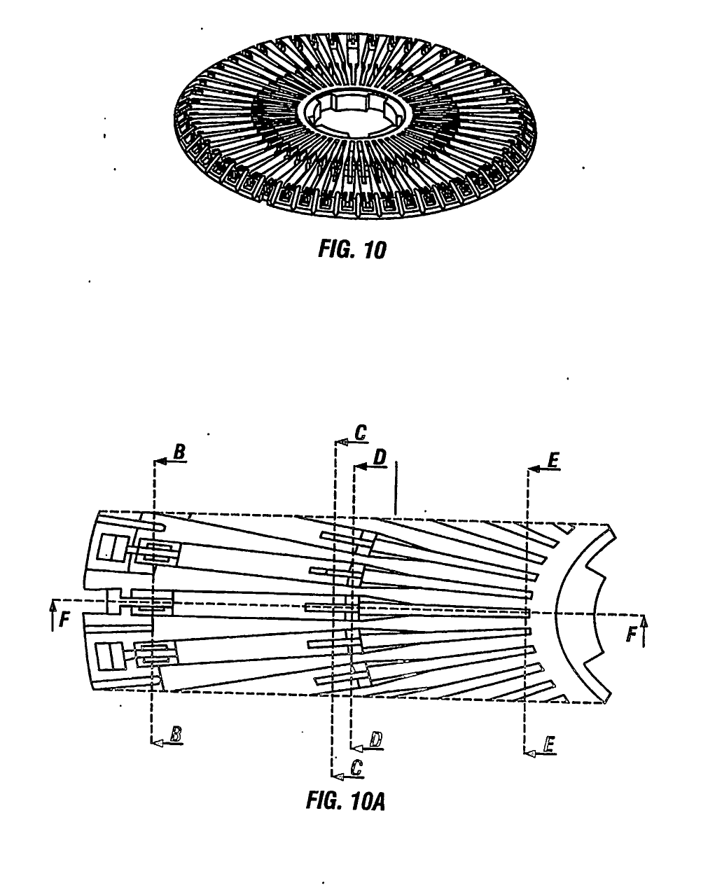

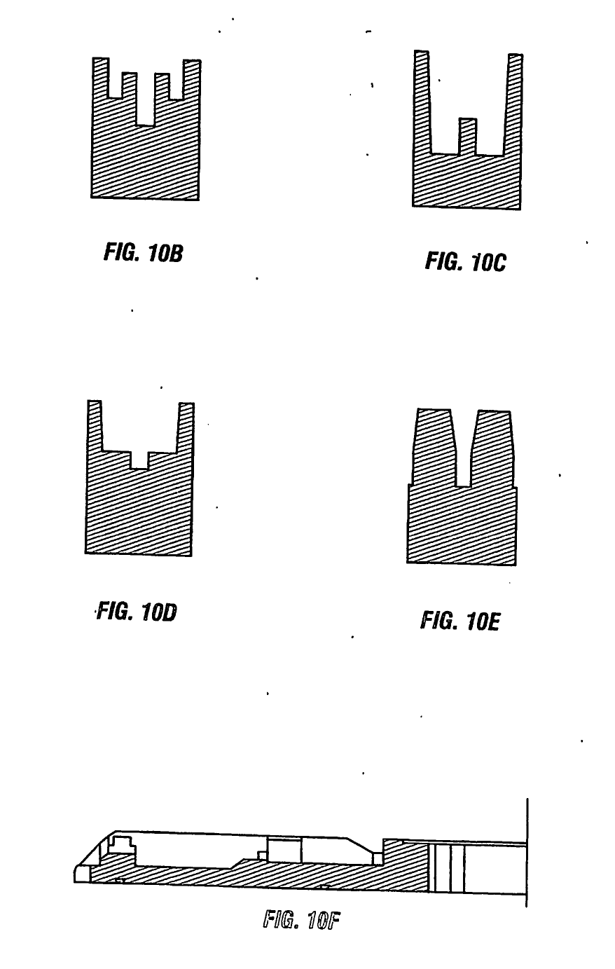

Referring now to FIG. 10, various cross-sectional views of one embodiment of a cartridge 242 according to the present invention is shown. It should be understood that the cartridge 242 may include a sterility barrier (not shown in FIG. 10 for ease of illustration). It should be understood that all measurements and dimensions shown in FIG. 10 are purely exemplary and other sizes may be used without departing from the spirit of the present invention. It should be understood that the dimensions near cross-section D are such that, in this embodiment, the penetrating member may be gripped or held in place by the cartridge, preventing the penetrating member from extending outward.

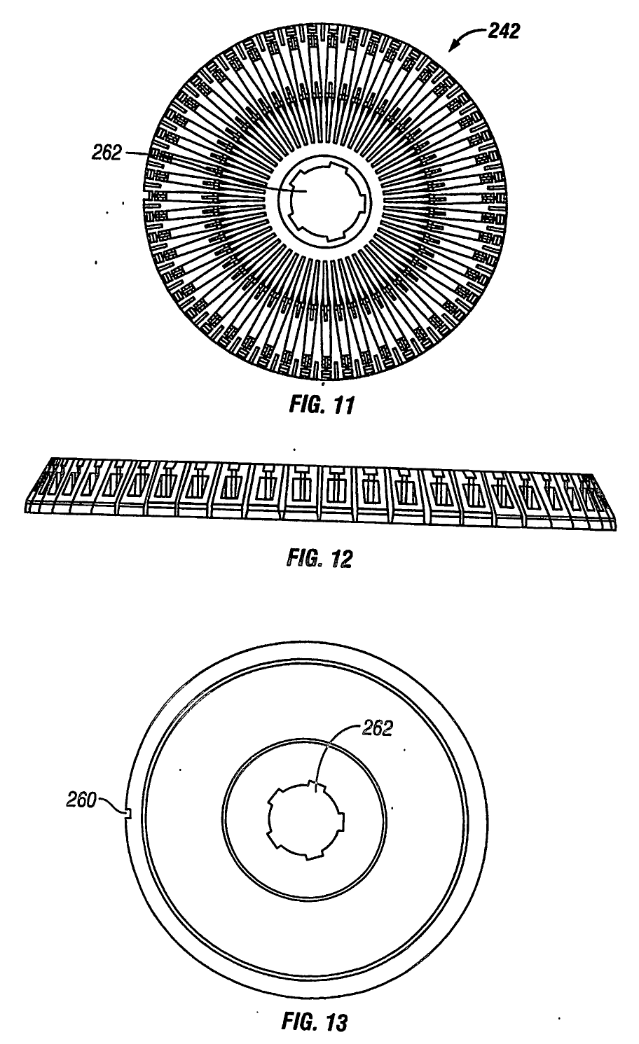

Referring now to FIGS. 11, 12, and 13, top down, side and bottom view of the cartridge 242 are shown. Again, all dimensions are purely exemplary and other sizes may be used or varied without departing from the spirit of the present invention. As seen the cartridge 242 may include a notch 260 on the outer perimeter (FIG. 13). This may be used for positioning of the cartridge 242 into the device. It may also be used for counting purposes, by tracking where this notch is, relative to the device 240, the number of penetrating members remaining may be calculated. The notch 260, in another device, may be be used to align the cartridge 242 on a rotating disc or surface, where rotation of the surface is used to track the position of the cartridge 242 and the number of penetrating members remaining. As seen in FIGS. 12 and 13, the inner diameter may include notches 262 for position purposes and for purposes of turning or rotating the cartridge 242.

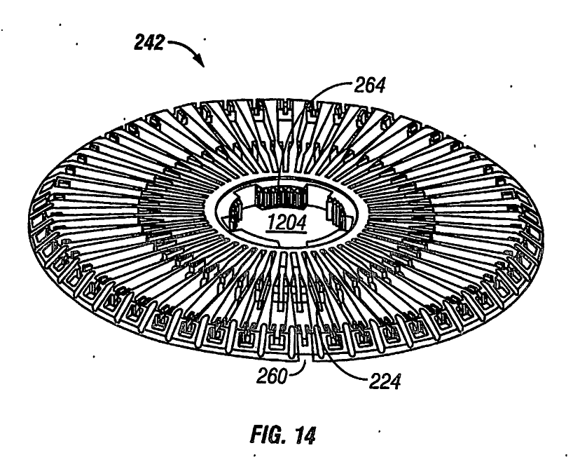

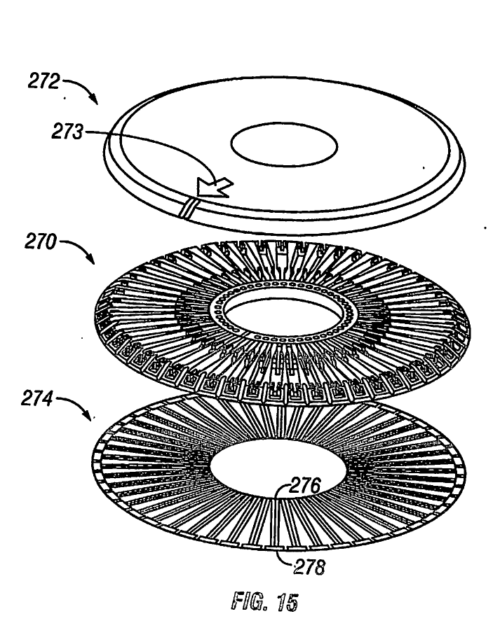

Referring now to FIG. 14, a perspective view of an embodiment of the cartridge 242 is shown. As seen in FIG. 14, penetrating members 224 are shown housed in the cartridge 242. FIG. 14 also shows an embodiment where a toothed surface 264 is included on the inner circumference of the cartridge 242.

Referring now to FIG. 15, a still further embodiment of a cartridge according to the present invention will be described. As seen in FIG. 15, this embodiment has a cartridge 270 with a sterility barrier 272 and a sensor layer 274. As seen FIG. 15, the sensor layer 274 has a plurality of leads 276 which extend from sensor 278. In one embodiment, these leads 276 extend to the inner circumference to connect to couplers (not shown) the sampling device 240. As seen on layer 272, an arrow 273, triangle, circle, dot, square, or other orientation marker may be on the layer 272 or in other embodiments on the cartridge 270 (or cartridge 242) or on the layer 274 to facilitate positioning by the user.

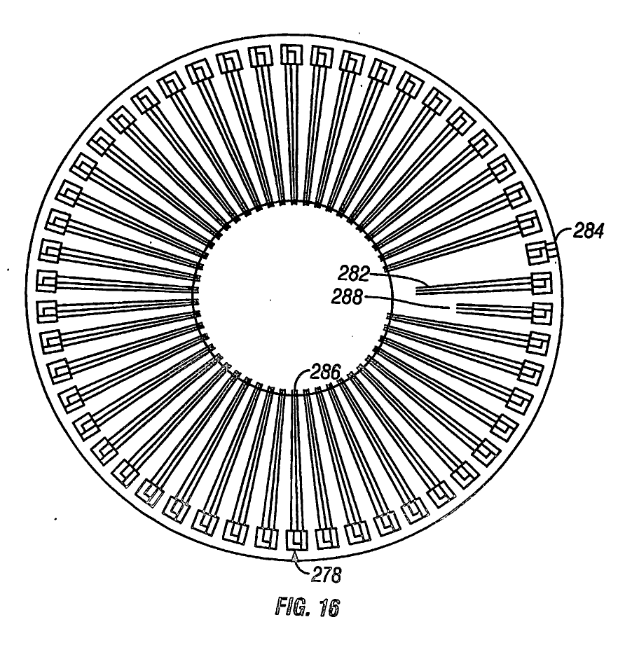

Referring now to FIG. 16, a top down view is shown of one embodiment of the sensor layer 274. It should be understood the leads 276 may also be configured to extend short of the inner diameter. For ease of illustration, various configurations are shown on the same sensor layer 274. It should be understood that the leads on the sensor layer 274 may all have the same configuration, any combination of the configurations shown herein, or other configurations. The leads 280 may extend roughly halfway while leads 282 extend further. They may then engage various contact pads (not shown) on the device 240 which can engage the leads. A still further embodiment has leads 284 that extend towards the outer circumference of the cartridge 242 to engage various contact pads or other connectors as known to those of skill in the art.

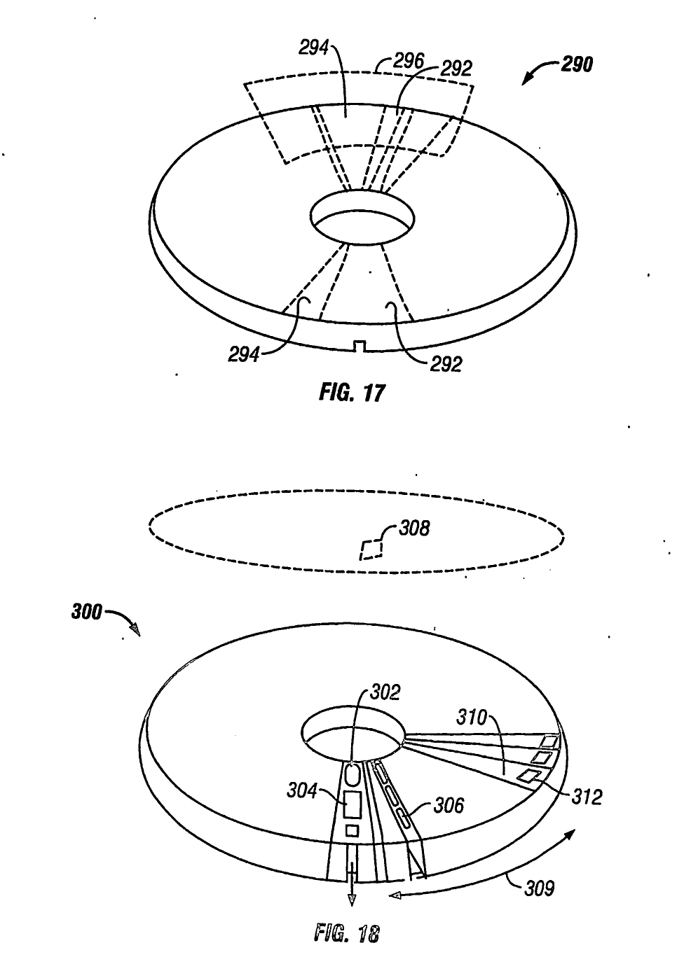

Referring now to FIG. 17, a still further embodiment of the present invention will be described. As seen in this embodiment, various visual markings may be made on the top surface and/or the bottom surface of the cartridge 290. FIG. 17 only shows the top side of the cartridge 290, however, these same markings may also be made on the underside. Although not limited to the following, these markings may be in the form of colors, numbers, bar codes, or other markings. The embodiment in FIG. 17 shows a region 292 that may be warning region indicating to the user that number of unused penetrating members are running low. The region 294 may be used to indicate that the penetrating members are almost all used. Although not limited to the following, these regions 294 may be color coded. The regions may also have certain properties that may be detectable by other sensors, such as but not limited to being magnetic or having other detectable properties. In some embodiments, the markings may be be on portions directly opposite of the active region since the window 296 may be on the side of the cartridge that is not the active side. These markings may cover only a portion of the cartridge or the entire cartridge may contain markings that provide information. Binary or other machine readable information may also be placed on the cartridge and then detected by a reader in the device 240 for detecting status information based on how the cartridge is positioned.

Referring now to FIG. 18, a still further embodiment of a cartridge according to the present invention will be described. For ease of illustration, multiple elements are shown on the cartridge 300. It should be understood that the cartridge 300 and others described herein may include some, none, or all of these features. This cartridge may include a dessicant 302 that may be included in the cartridge 300 to absorb moisture that may enter therein. Dessicant 302 may be in the form of a pellet or other embodiment that is a separate element from the cartridge. In other embodiments, the dessicant 304 may be printed, deposited, or otherwise integrated to the cartridge 300. The dessicant 306 may be on the side surfaces of cavities in the cartridge 300. A still further embodiment has the dessicant 308 attached by some technique such as, but not limited to printing, adhering, forming, or other wise integrating the dessicant 308 to some material coupled to the cartridge, such as but not limited to the sterility barrier. These techniques may also be used to attach these dessicants to the cartridge or even the sensor layer. They may also be coupled to a separate layer of material attached to the cartridge that only covers a portion of the cartridge.

A still further feature of the cartridge 300 is to include a separate test that the cartridge 300 may be rotated to if the processor on the device detects that the analyte readings from a regular test requires a specialized test to be conducted. The cartridge 300 will be rotated as indicated by arrow 309. As a nonlimiting example, if glucose readings are detected outside a desired range, a processor may recommend a HB1AC test be conducted. The cartridge 300 may then be rotated to designated position to align a cavity 310 with a sensor 312 for this test. This rotation may occur by mechanical actuation or by electric powered rotation of the cartridge. In this embodiment, each cartridge may have three HB1AC tests. Other cartridges may have other numbers or they may have other test areas in place of the HB1AC tests.

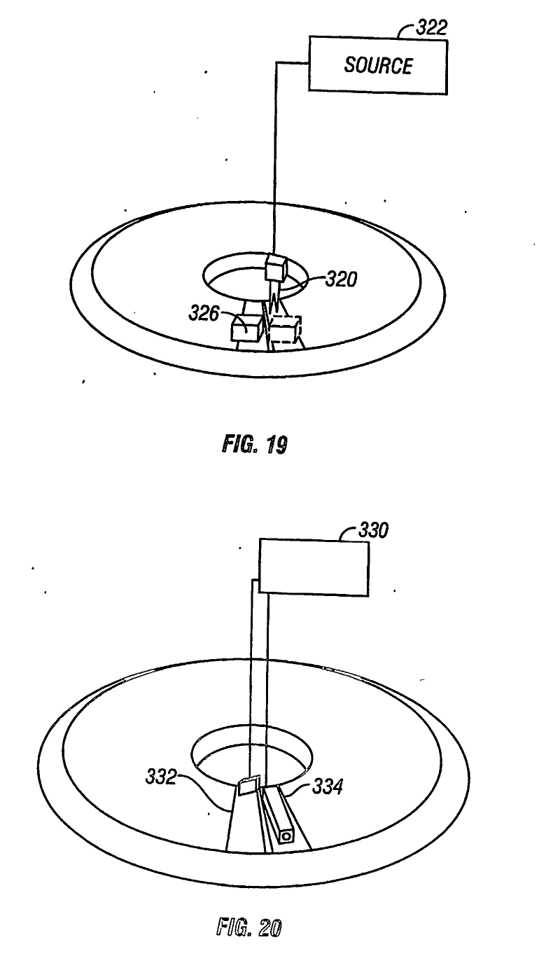

Referring now to FIG. 19, a still further embodiment of penetrating member coupler will now be described. In this embodiment, the coupler 320 is linked to an electrical source 322 that can be controlled to release a charge to sterilize a penetrating member coupled to the coupler 320. Thus, prior to use, the coupler 320 can sterilize the penetrating member before the member is driven into tissue.

FIG. 19 also shows a heating element 326 that may be used to melt a portion of the cartridge after the penetrating member has been actuated. This will hold the penetrating member in place and prevent the sharp from being released from the cartridge. The heating element 326 may be positioned at a cavity adjacent or in other embodiments, simply away from the active cavity. In ether embodiments, the heating element 326 (shown in phantom) may be positioned over the activity cavity.

Referring now to FIG. 20, a still further embodiment of the present invention may use a processor 330 to detect the amount of force used a cutter 332 or a punch 334 to pierce the sterility barrier on the cartridge. Although not limited to the following, the amount of force may be used to detect if the cavity on the cartridge is a used and the sterility barrier has already been breached.

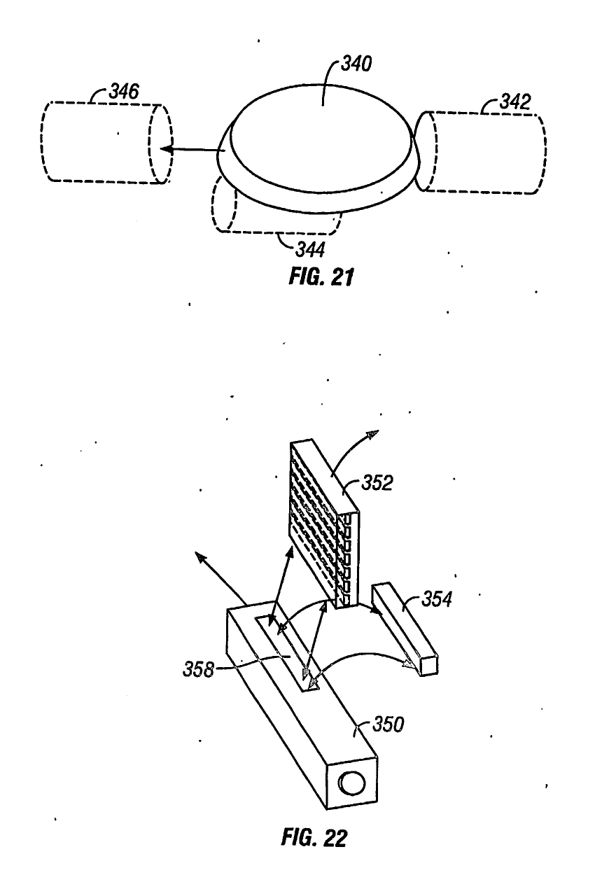

Referring now to FIG. 21, a still further embodiment of the present invention will now be described. As seen in FIG. 21, one embodiment of a cartridge 340 according to the present invention may not have a opening in the center like those cartridges shown in the previous figures. FIG. 21 also shows various receptacles 342 and 344 shown in phantom. In this embodiment, the penetrating members are not parked in the cartridge after use. They may be actuated and then released. They may fall into container 344 through a slot in the bottom of the cartridge. Or they be dumped into the receptacle 342 after use. In some configurations, the user may simply grab the used penetrating member, pull it out of the cartridge, and deposit it into a receptacle. In a still further embodiment, a used penetrating member may be ejected from the cartridge during the process of loading a new penetrating member. The used penetrating member is ejected out into a receptacle 346.

Referring now to FIG. 22, another embodiment of the present invention will now be described. FIG. 22 shows a method for use with a penetrating member driver 350 which is brought together with a multiple penetrating member cartridge 352 or single cartridge 354 only during use. After use, the cartridge and driver are separated for ease of storage, sterility, or other reasons. As seen, the cartridge 352 may be inserted into slot 358. Although not limited to the following, the penetrating member driver may be any of those described herein or the applications incorporated by reference. The driver may also be any of those described in commonly assigned, copending U.S. patent application Ser. No. 10/425,815 (Attorney Docket No. 38187-2663) filed May 30, 2003, fully incorporated herein by reference. In one embodiment, a case may be provided to store both the driver and the cartridge, or just the driver, or just the cartridge.

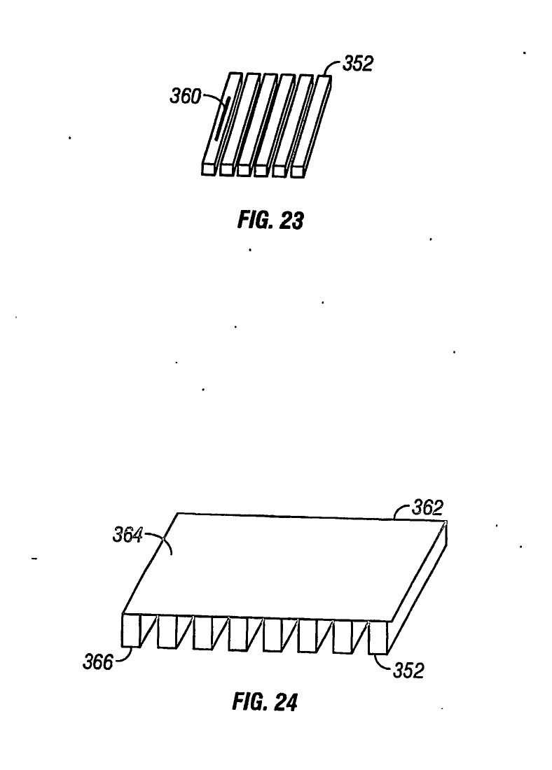

Referring now to FIG. 23, a plurality of the cartridges 352 are shown. Each may contain a penetrating member 360. These cartridge 352, in this embodiment, may be substantially equivalent to on cavity on the cartridge 242. The cartridge 352 may include an area that has sufficient interference fit to grip a penetrating member and it may have an elongate opening on one side, such as but not limited to top or bottom, to allow a penetrating member coupler to engage the penetrating member 360 therein. It should be understood, that in some embodiments, only proximal and distal end openings are provided.

Referring now to the embodiment shown in FIG. 24, a plurality of cartridges 352 are coupled together by some layer of material 362. They may use a sterility barrier to couple them together. In another embodiment, a mold plastic or polymer may be used to couple the cartridge 352 together. The cartridges may be designed to allow access from the top as indicated by arrow 364 or from the bottom 366.

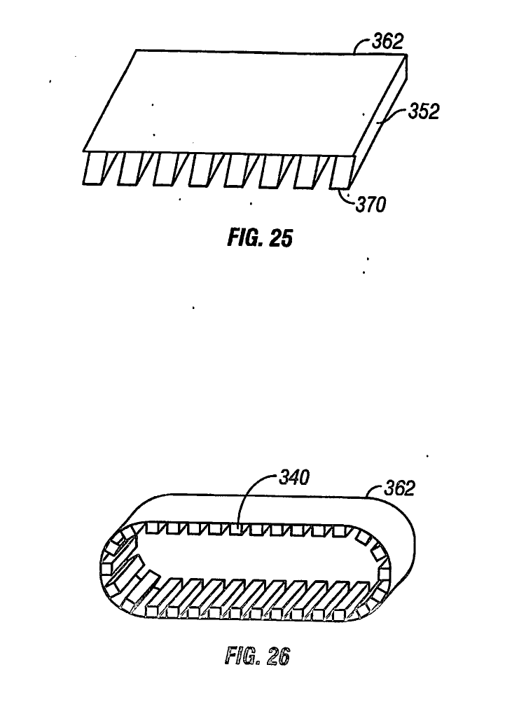

FIG. 25 shows a still further embodiment where a distal portion 370 is not covered by the sheet 362. In this embodiment, this is where a punch would come down and punch down material covering the front.

FIG. 26 shows an embodiment where the cartridges may be coupled into a configuration as shown.

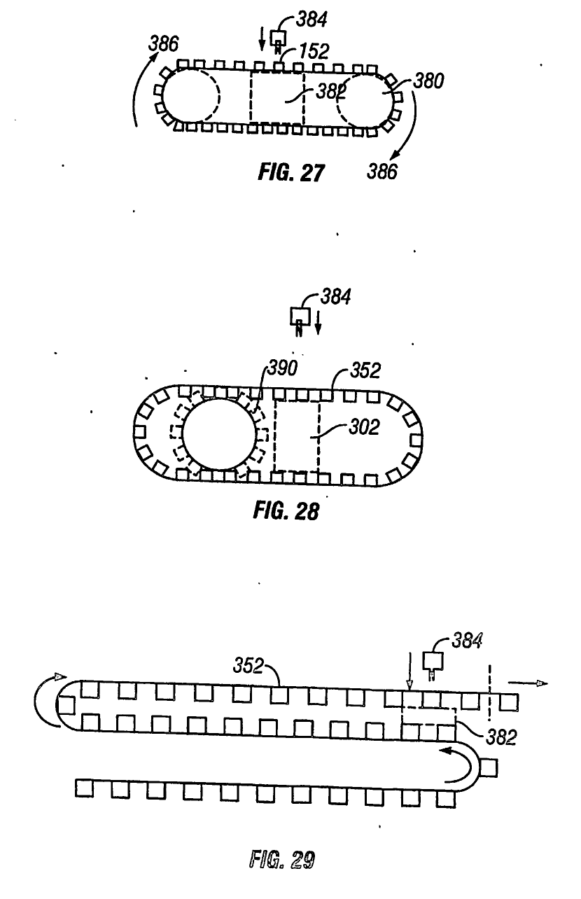

Referring now to FIGS. 27 and 28, embodiments of cartridges 352 coupled together is shown used with rollers 380 and a support surface 382 over the active region. A penetrating member coupler 384 may be used to engage penetrating members inside the cartridge 352. The cartridges may be rotated as indicated by arrows 386 to bring cartridges into alignment with the coupler 384.

FIG. 28 shows a similar embodiment where the roller 388 has teeth 390 to engage spaces between the cartridges 352. The embodiment in FIG. 28 allows the coupler 384 to pierce through the material 362 to engage the penetrating member therein. In one configuration, the punch and opening of the sterility barrier is very similar to those techniques used for cartridge 242.

FIG. 29 shows a still further embodiment where the cartridges 352 are in an elongate strung out configuration that may be layer on top of each other or otherwise configured to efficiently store the tape of cartridges 352. The embodiment of FIG. 29 may include a cutter at the position indicated by dotted line 392 where the material 362 is cut and the used cartridge 352 may be discarded. The cutter (not shown) may any of those known to those of skill in the art.

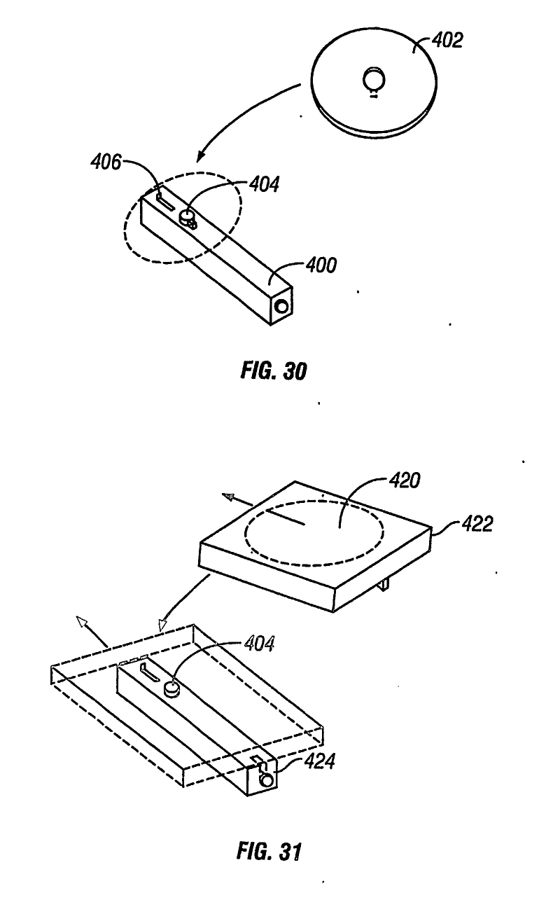

Referring now to FIG. 30, a still further embodiment is shown where a penetrating member driver 400 may be coupled to cartridge 402 containing a plurality of penetrating members. The cartridge 402 may snap on the holder 404 and be held in place similar to that technique used by amaray or cases used to DVDs or CDs. A penetrating member 406 may be included.

FIG. 31 shows a still further embodiment where disc 420 holding the penetrating members are contained inside a housing 422. The housing 422 may be coupled to a penetrating member driver 424. Any of the drivers disclosed herein may be used for the driver 424. Again, the configurations here may teach a method of use where the penetrating members and drivers are brought together only when the sampling is about to occur.

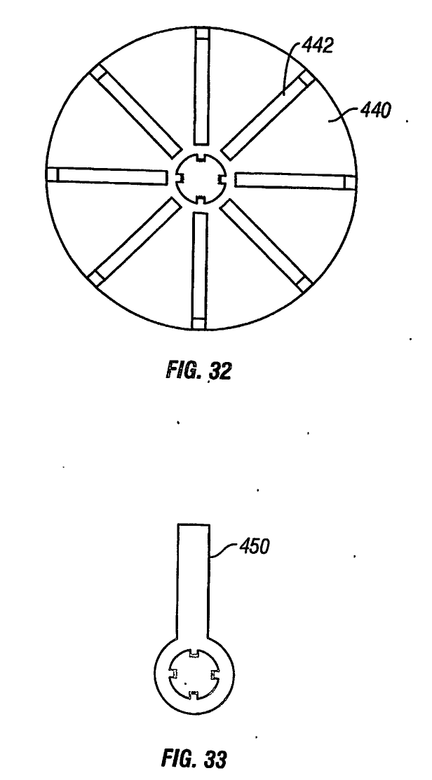

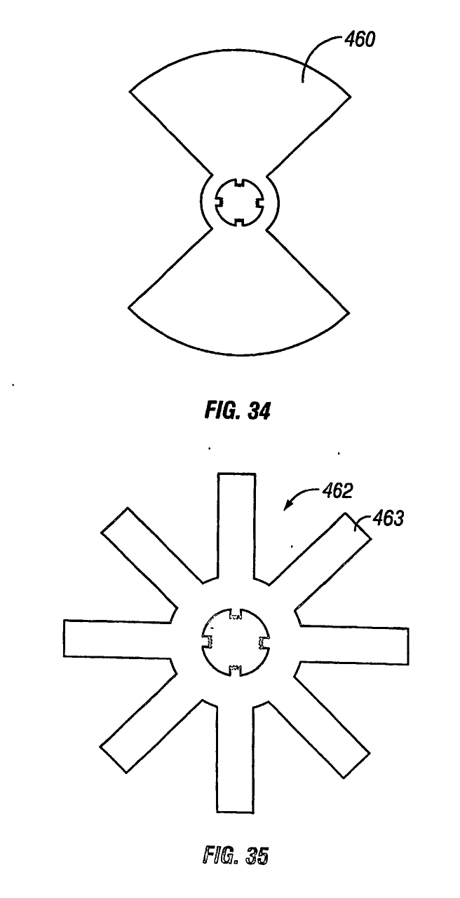

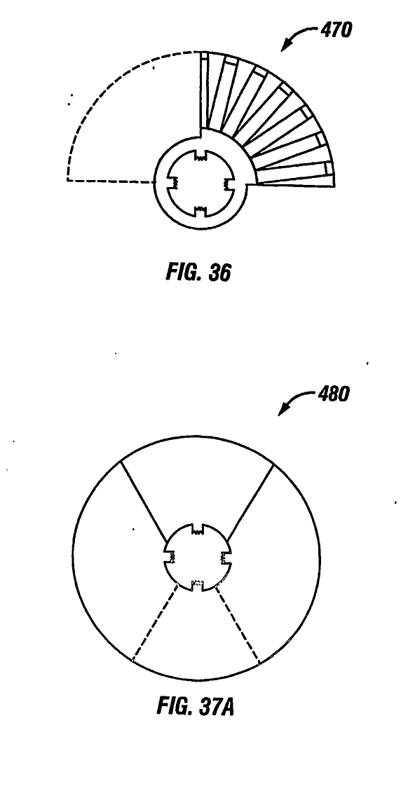

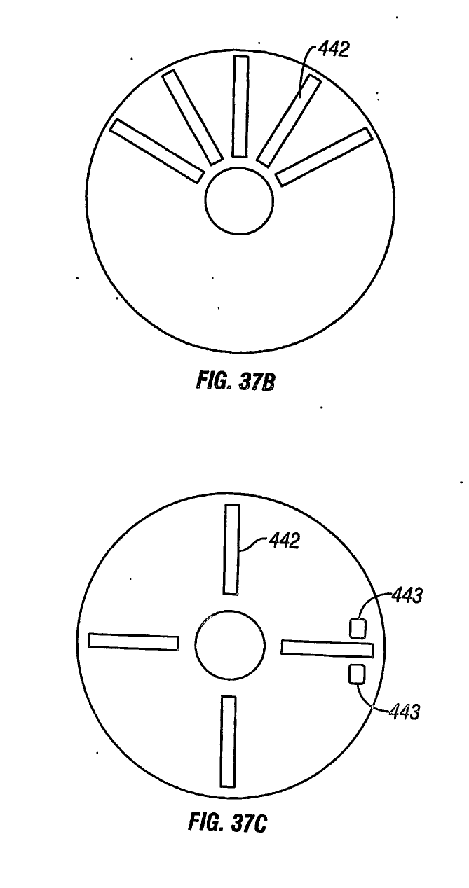

Referring now to FIGS. 32 through 37 show embodiments of cartridges for use with device 240. FIG. 32 shows a full circular cartridge 440 with a reduced number of cavities 442 spaced apart on the cartridge. FIG. 33 shows a single cartridge 450 holding a single penetrating member. FIG. 34 shows a cartridge 460 having two wedge configuration. FIG. 35 shows a cartridge 462 having a single cavity per finger 463. Of course in other embodiments, there may be more than one cavity in each finger. The entire cartridge may also be a partial circular configuration (half-circular, quarter circle, wedge, etc . . . ). FIG. 36 shows an embodiment of a cartridge 470 with a half circular or ¼ circular or wedge configuration. FIG. 37A shows a cartridge 480 that may have portions, such as but not limited to quarters, thirds, or other fractions, on it that are colored or otherwise marked to show, as a nonlimiting example, number of penetrating members remain or the type of analyte test held therein. FIG. 37B shows another embodiment wherein penetrating member cavities are only disposed over a wedge portion of the cartridge. In FIG. 37C, the penetrating member cavities are very loosely dispersed over the cavity. Some embodiments may have one or more analyte detecting members 443 positioned to receive body fluid.

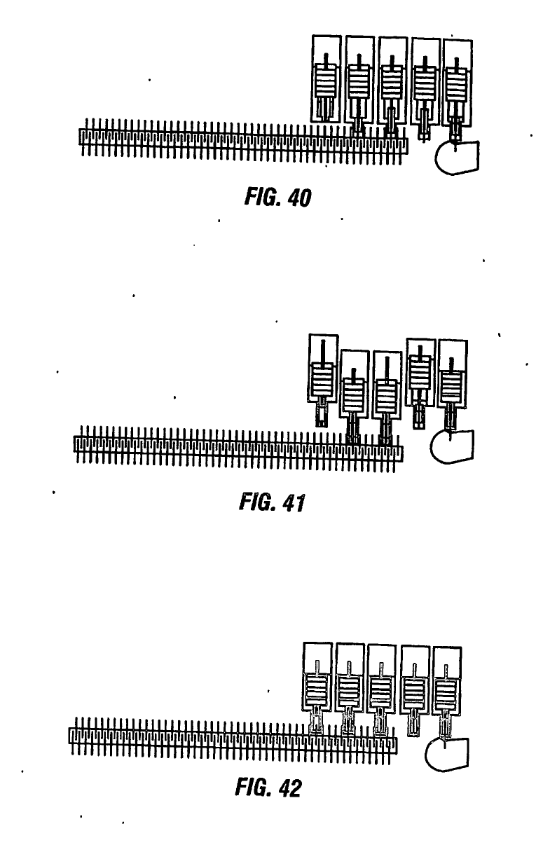

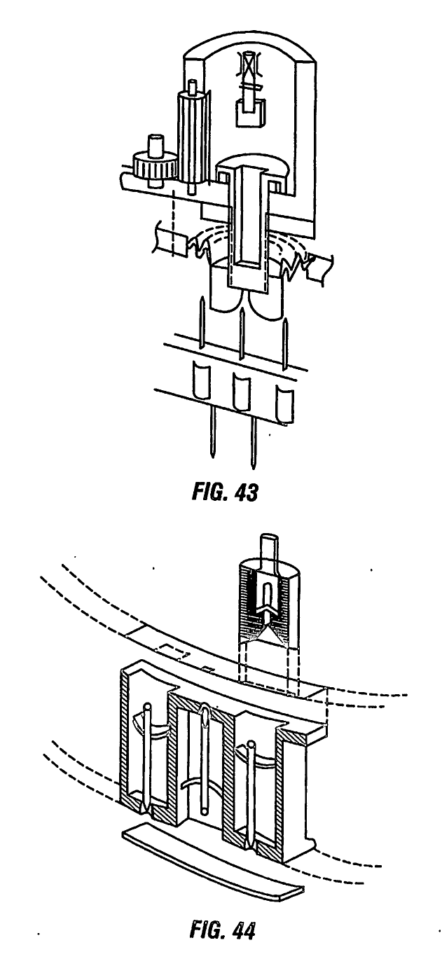

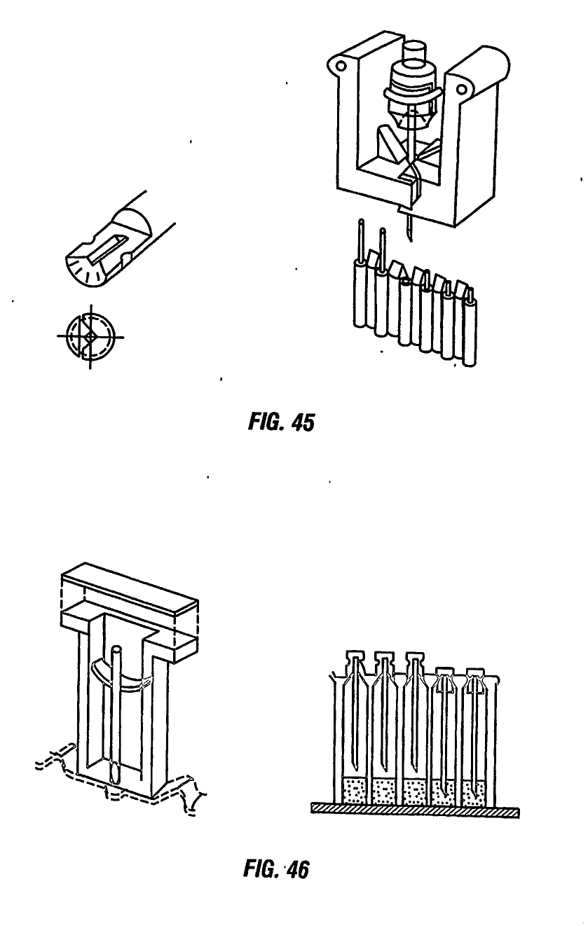

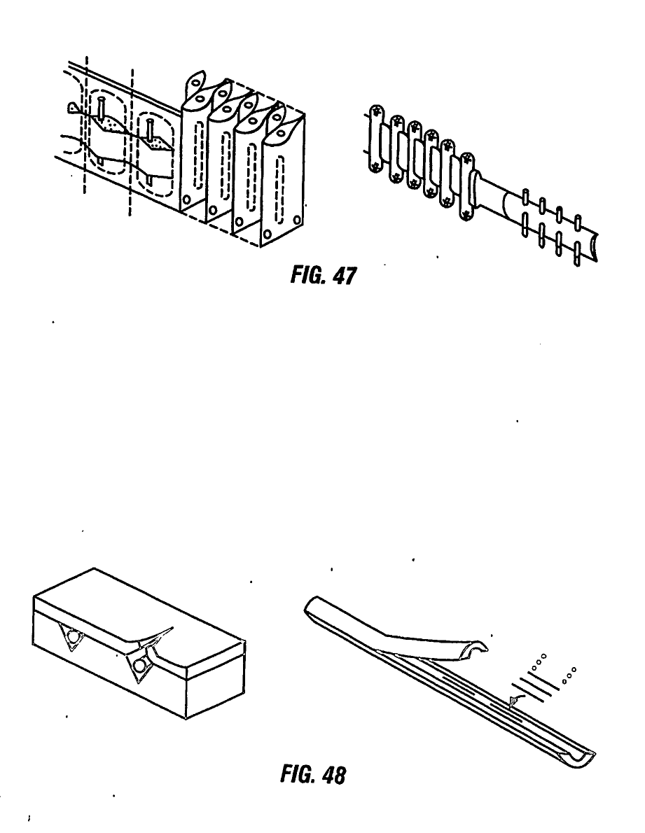

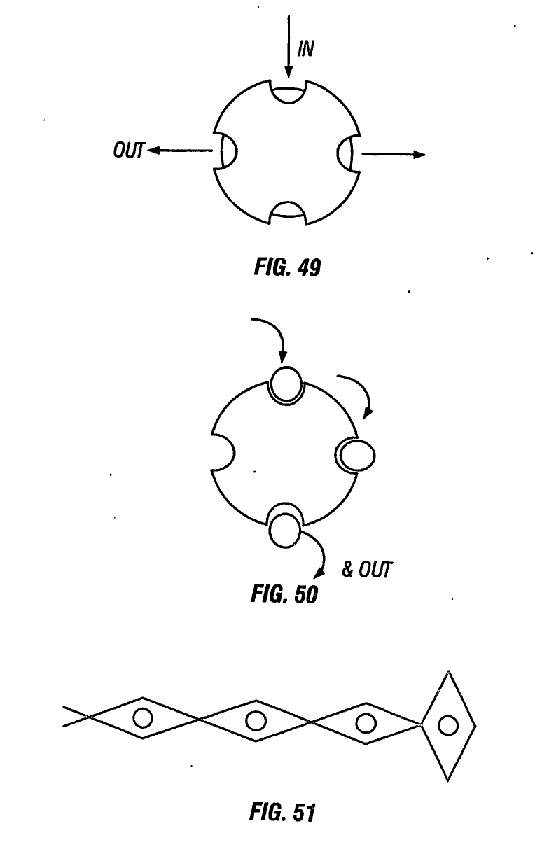

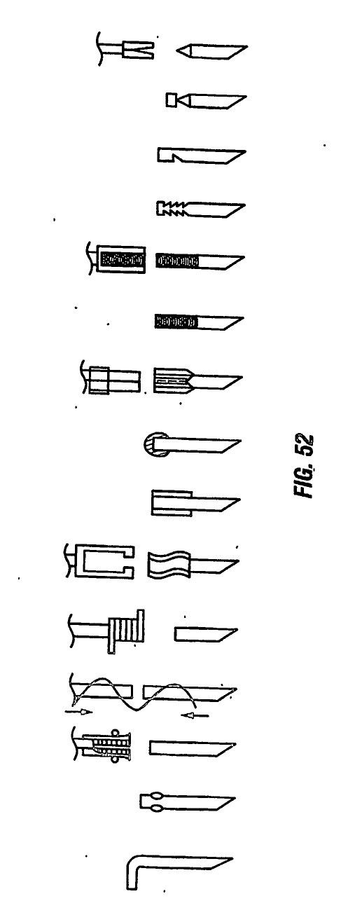

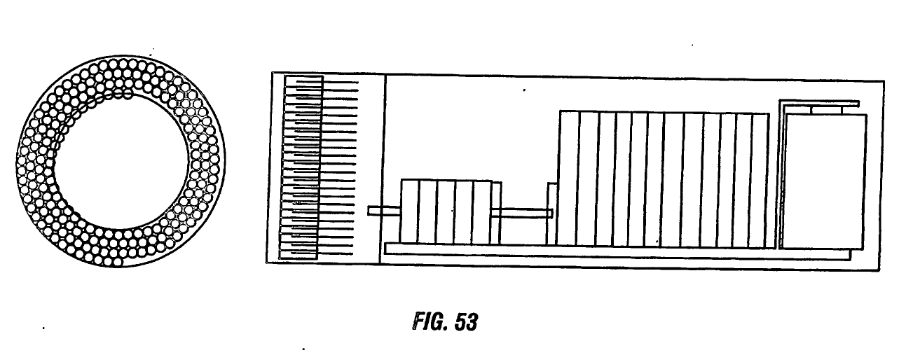

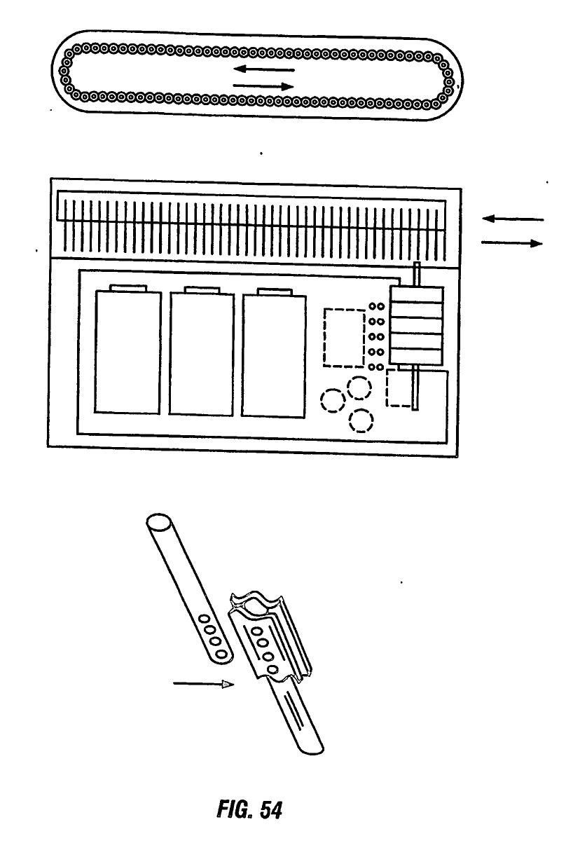

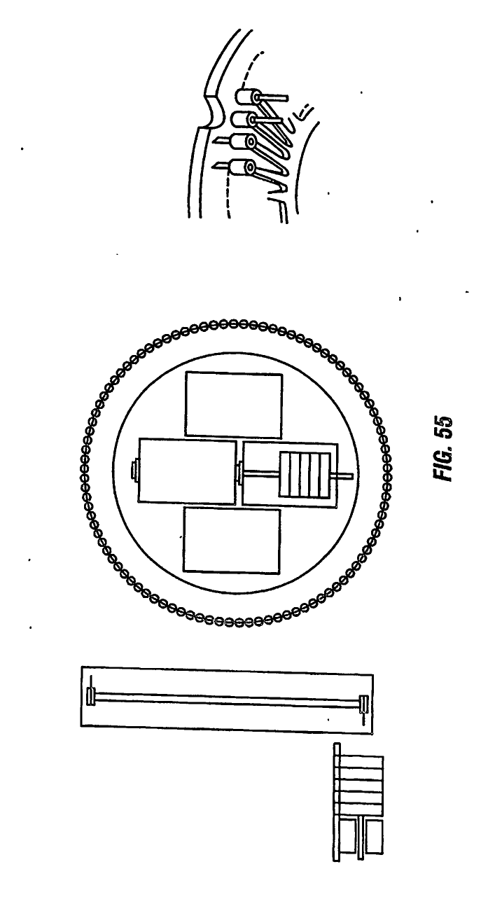

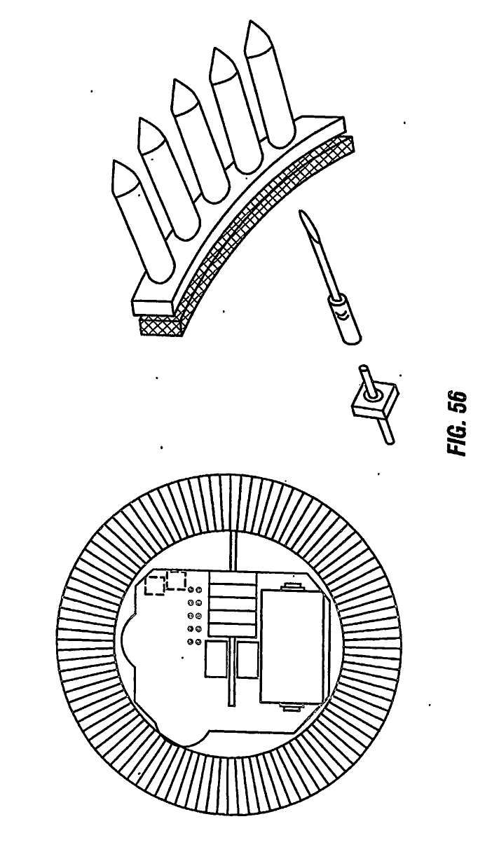

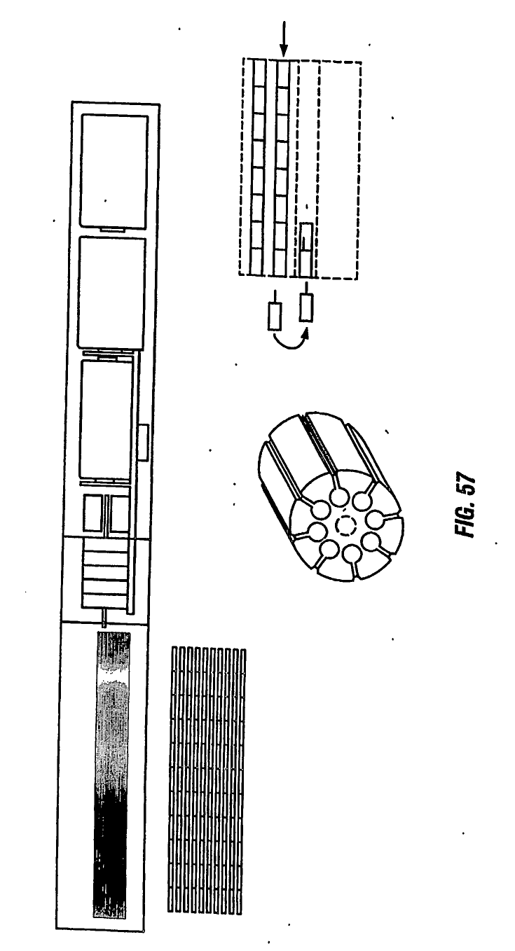

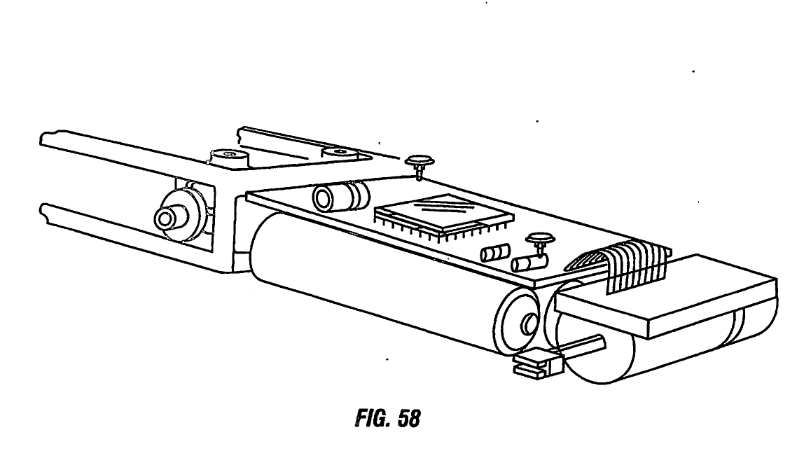

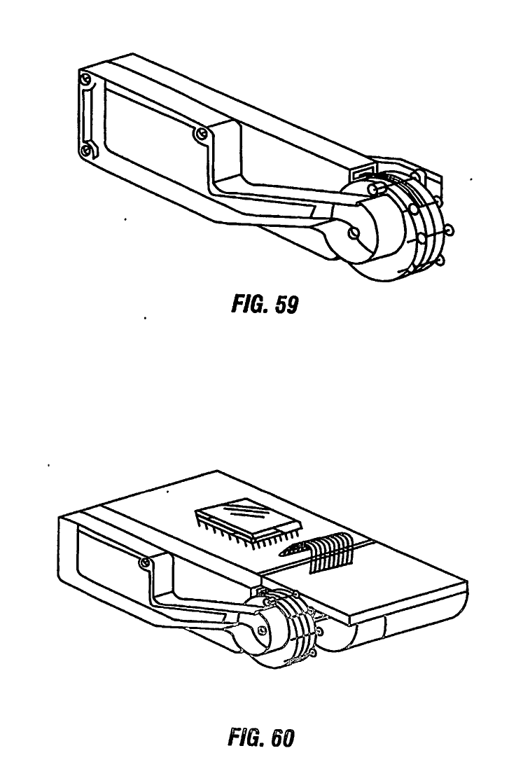

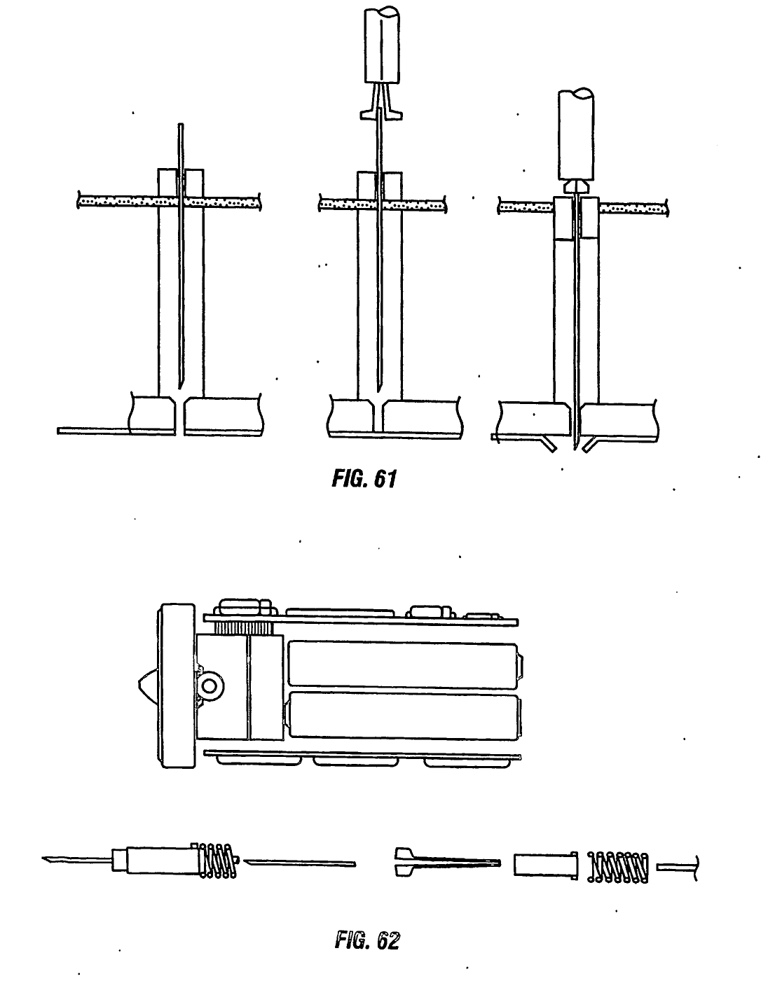

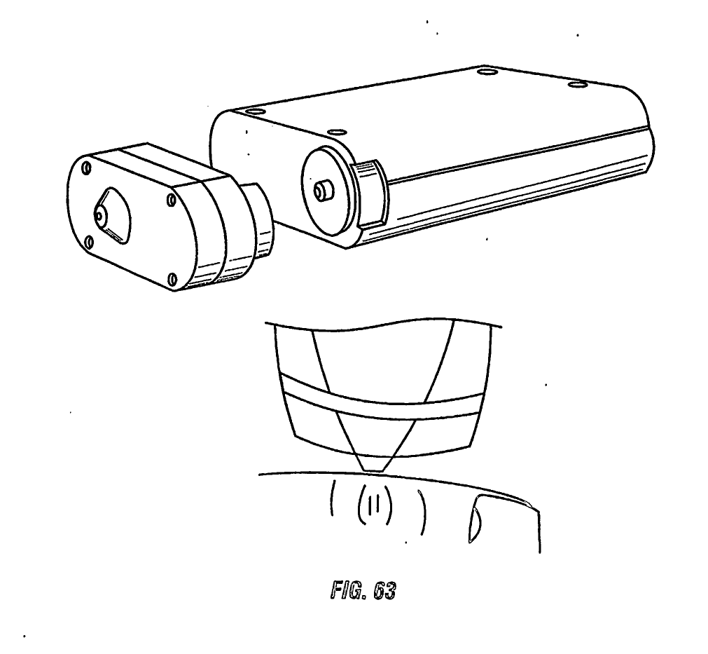

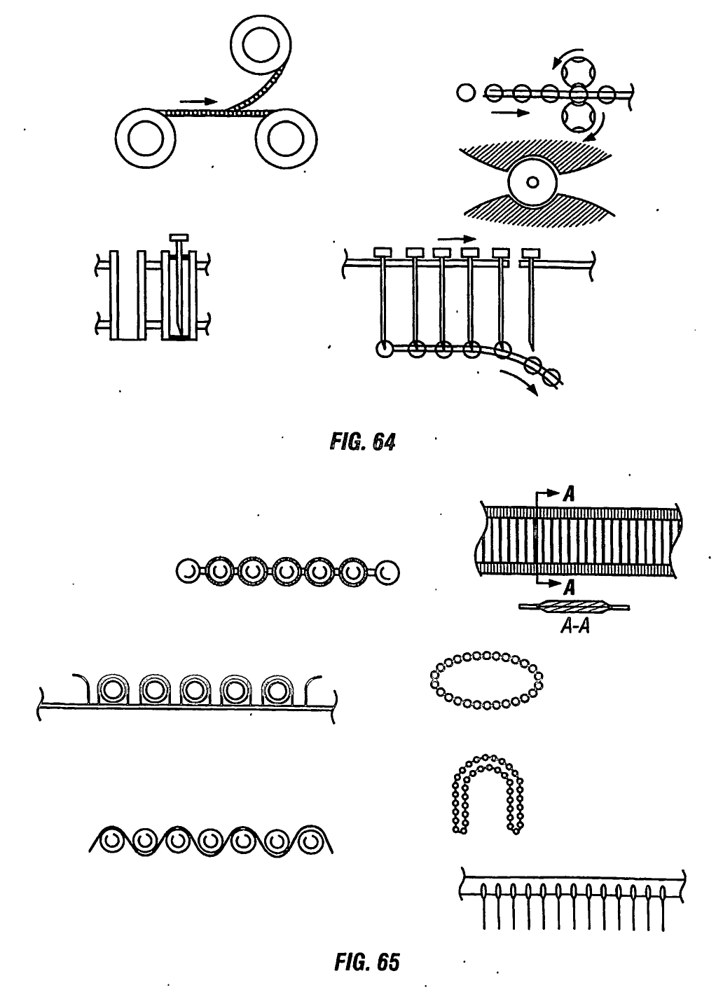

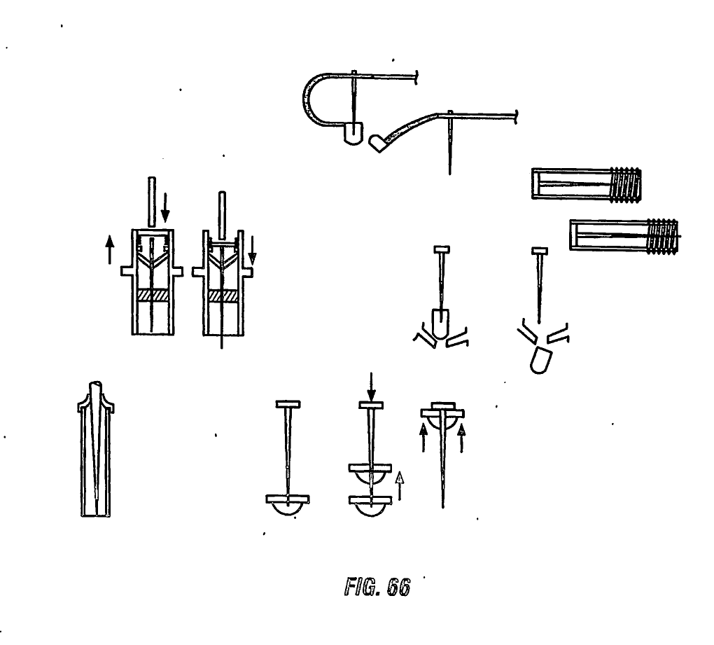

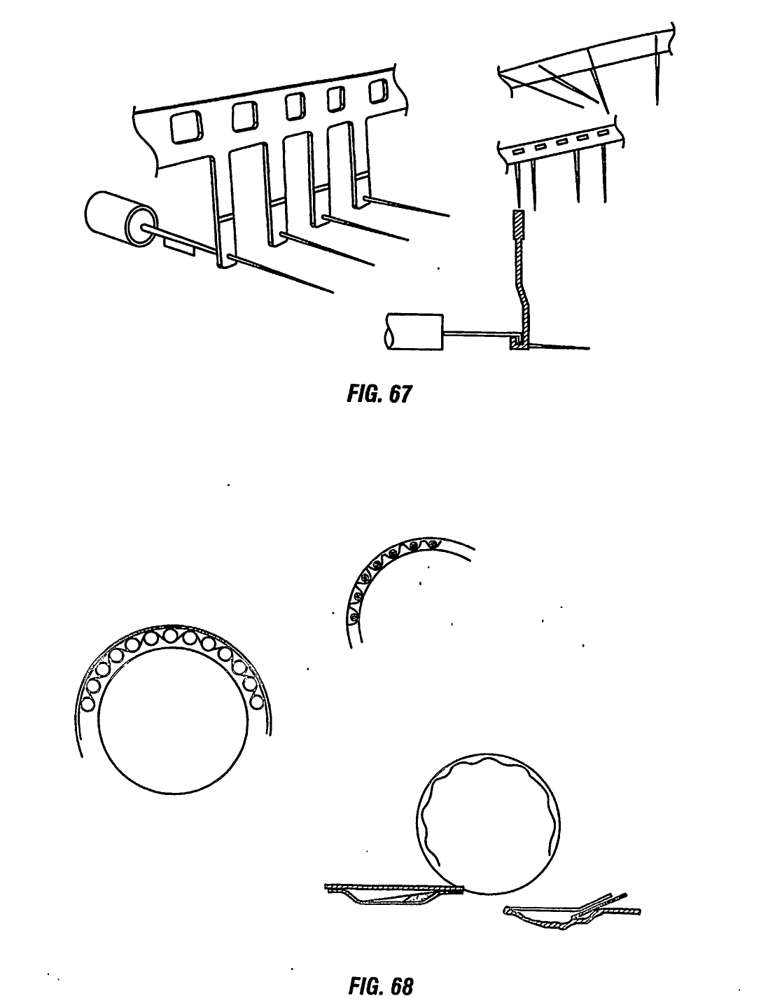

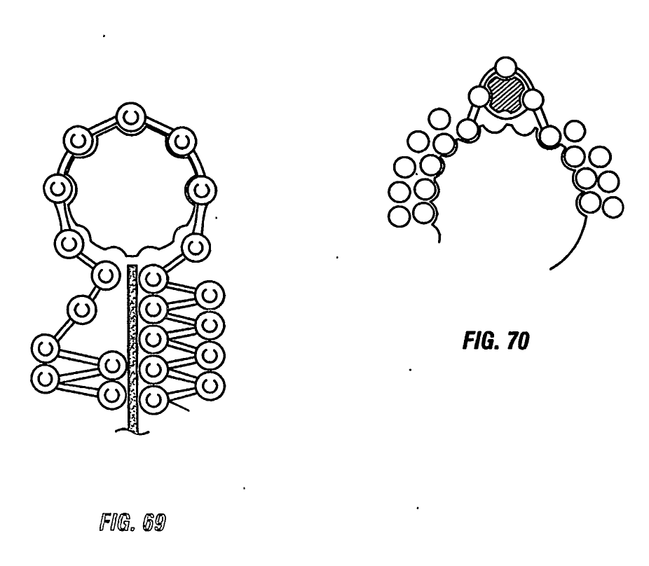

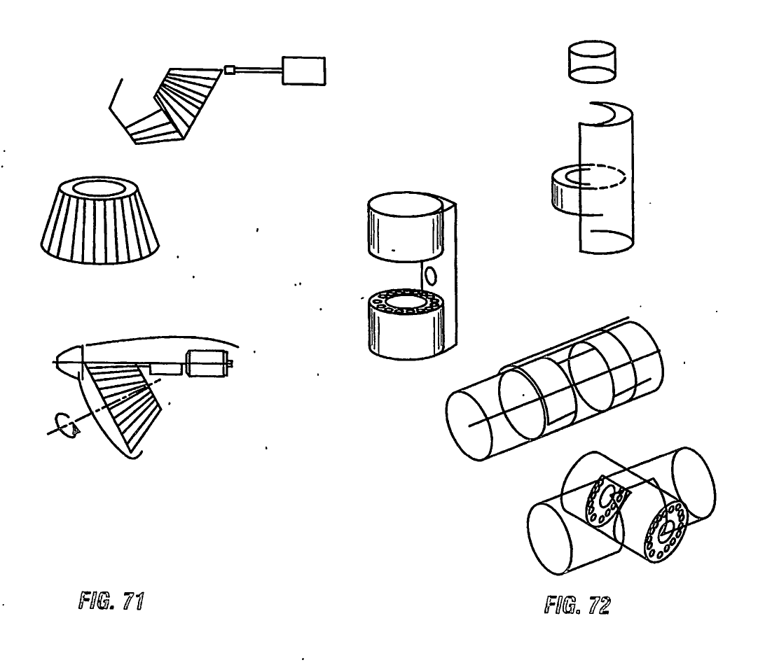

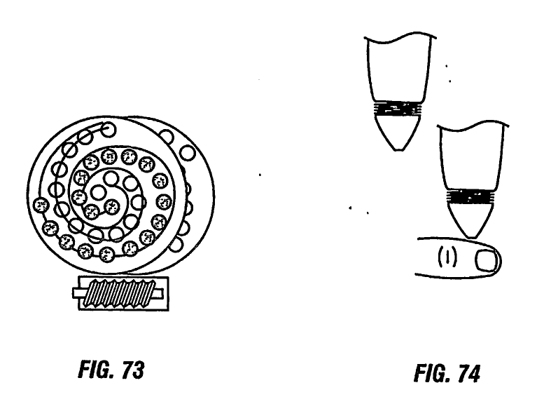

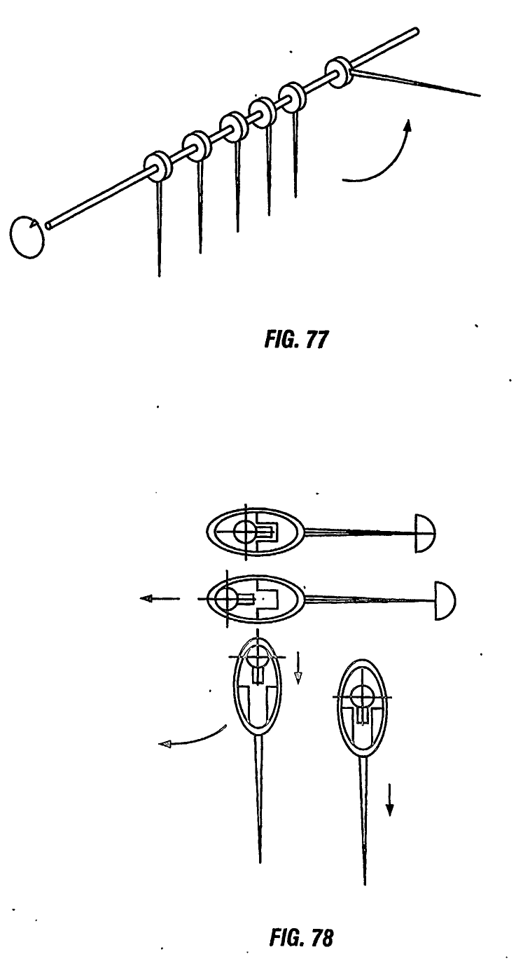

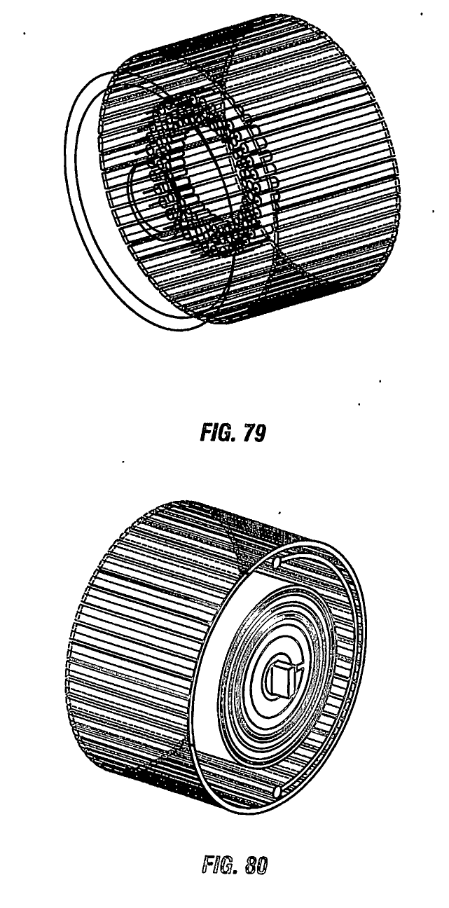

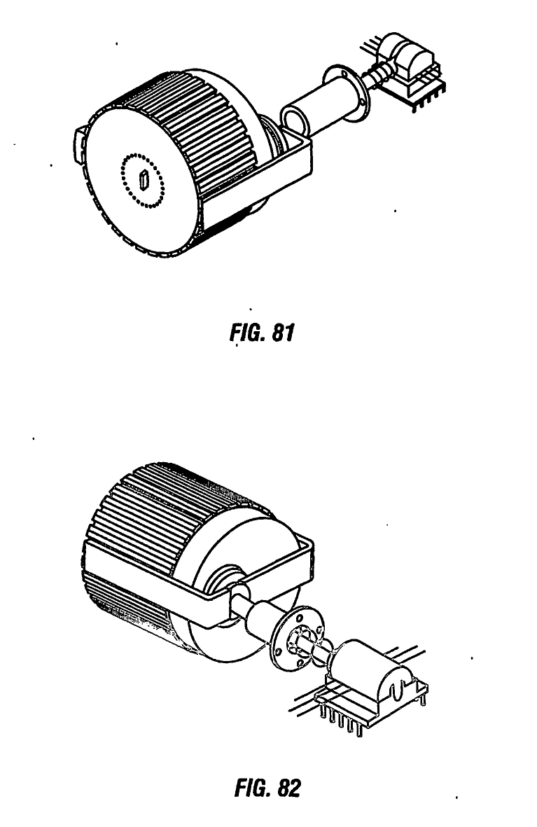

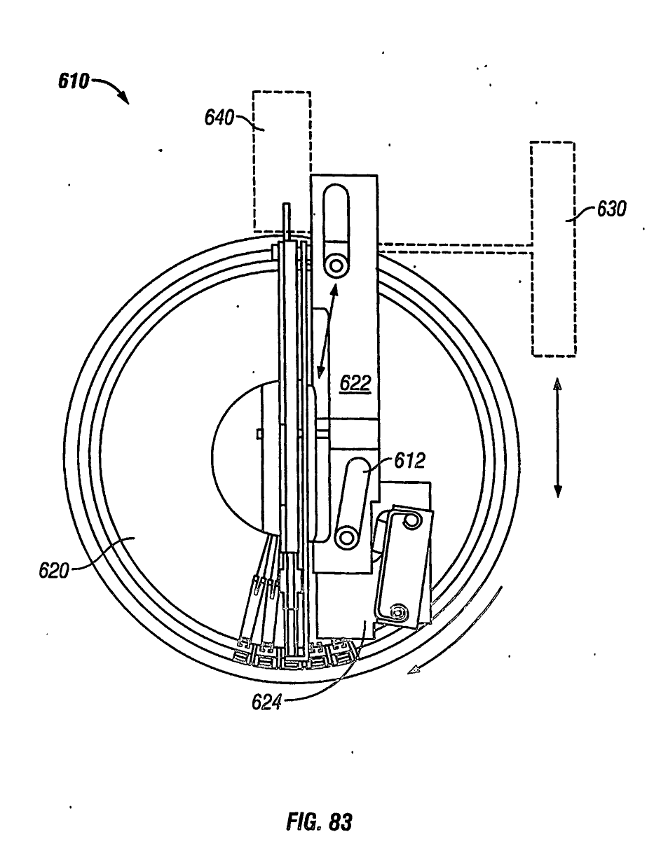

FIGS. 38 to 82 show still further embodiments of the present invention.

2685

Referring now to FIG. 83, another aspect of the present invention will now be described. One embodiment of a cartridge indexing device 610 is show. The indexing device 610 include a cam groove 612. It moves a cutting element (see FIG. 88) that opens a sterility barrier covering the cartridge 620. As seen in FIG. 83, the path of pusher 622 is at a diagonal as indicated by arrow 624. Thus, as the pusher 622 is advanced outward, it both cuts the sterility barrier and it rotates the cartridge 620 since the pusher 622 is moving at a diagonal. A slider 630 may be use by the user to advance and move the pusher 622. A penetrating member driver 640 may be coupled drive the penetrating member gripper 650.

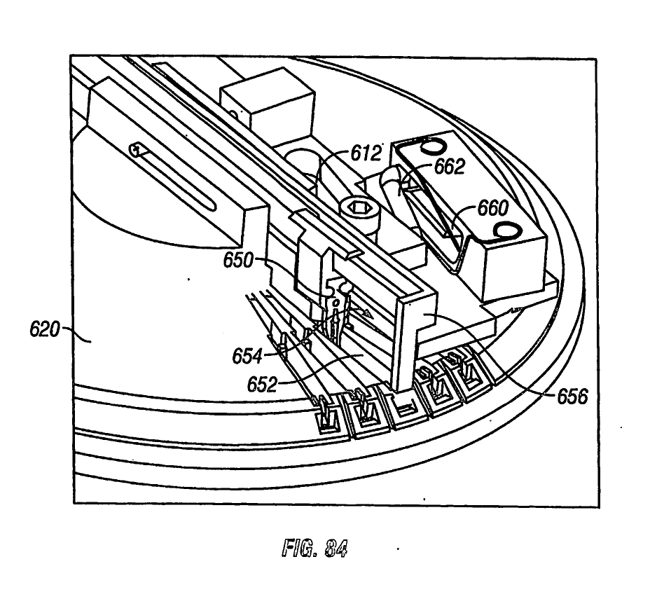

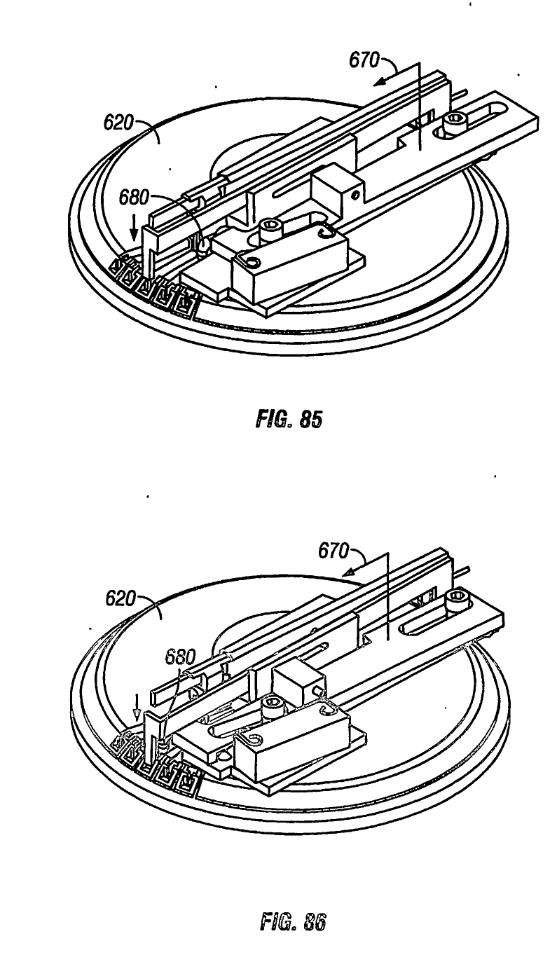

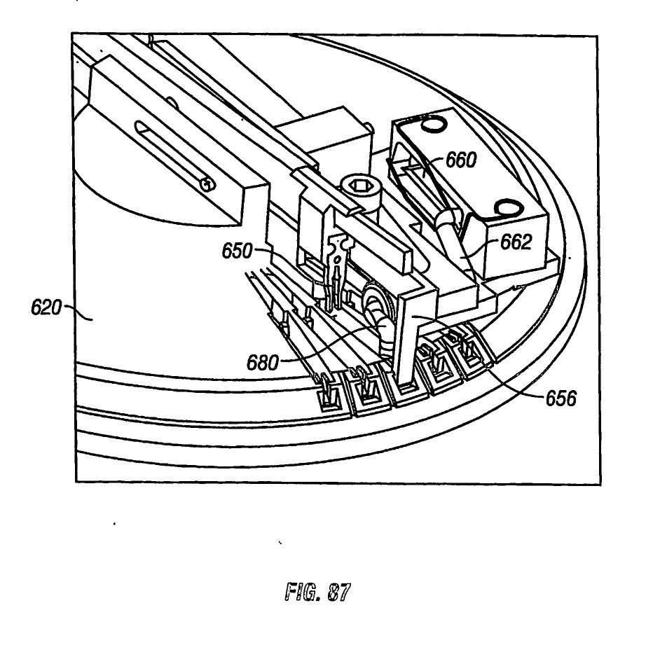

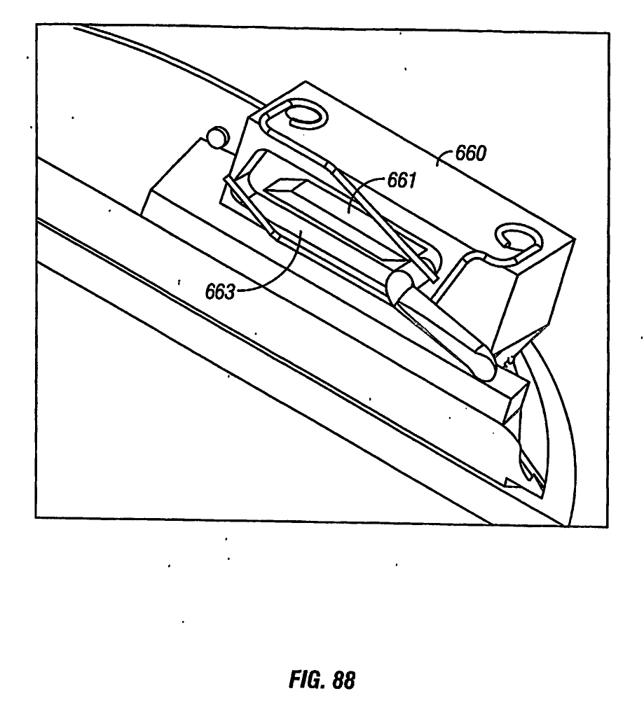

FIG. 84 shows the penetrating member gripper 650 more clearly. This is the start and end position. As seen, the gripper 650 is in the cavity 652. The gripper 650 may be advanced outward as indicated by arrow 654 to direct the penetrating member into tissue. The punch 656, used to break the barrier covering the penetrating member exit, is in an up position to allow the penetrating member to exit. As seen in FIG. 84, a second cam 660 is shown. The follower 662 is coupled to the sterility barrier cutter that cuts the barrier from the top and also rotates the cartridge 620. This cam 660 lowers the cutter on the forward stroke (to cut barrier and rotate cartridge) and raises it on the return stroke (so it does not rotate the cartridge back).

FIGS. 85 and 86 show the pusher 622 being advanced as indicated by arrow 670. FIG. 86 shows the pusher 622 fully advanced. As seen, the punch 656 has dropped to open the front sterility barrier. The cutter 680 has moved from a rear of the cavity (FIG. 85) to a front of the cavity (FIG. 86).

FIG. 87 shows a side view of the same position of the pusher 622 as that in FIG. 86. As seen, the punch 656 has dropped, the cutter 680 has finished its stroke. The follower 662 is in the top groove of the cam 660. When the follower 662 drops to the bottom groove, the cutter 680 will be raised. As seen, the gripper 650 is in a raised position and allows the cartridge 620 to rotate.

FIG. 88 is a close-up view of the second cam 660. It has a top groove 661 and bottom groove 663. The follower 662 travels through the grooves and flips the cutter 860 between up and down positions. In one embodiment, the follower 662 is in the top groove 661 in the forward stroke and in the bottom groove 663 in the backward stroke.

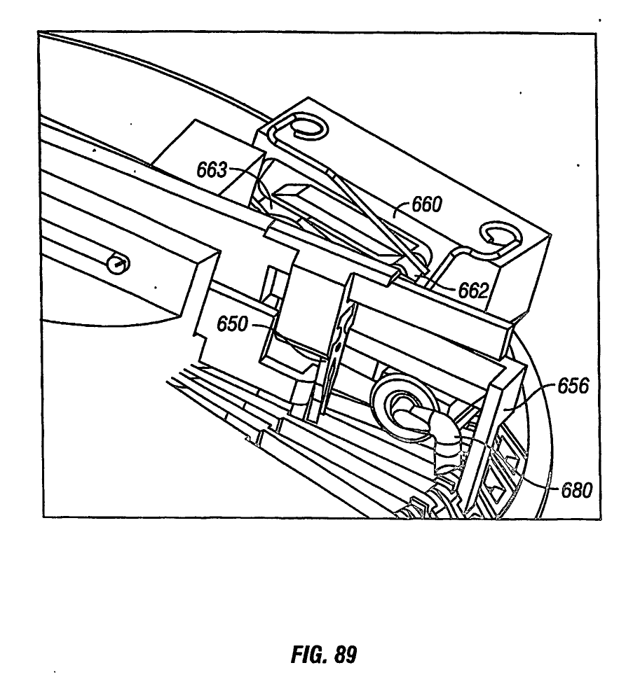

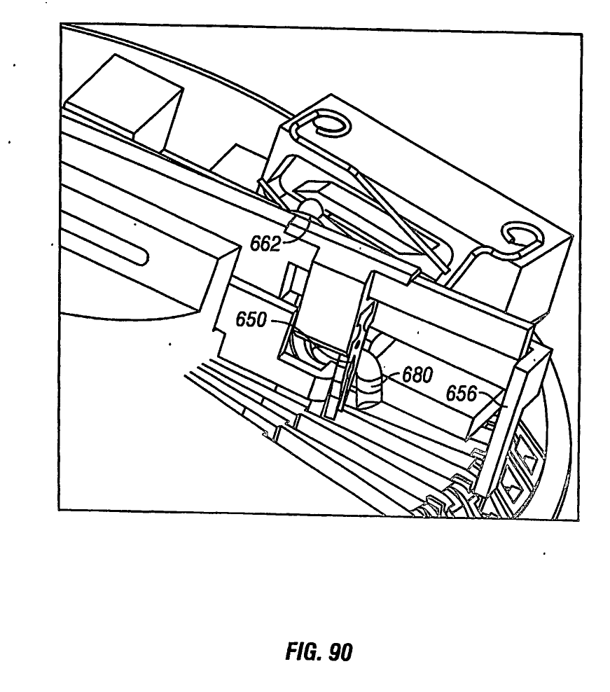

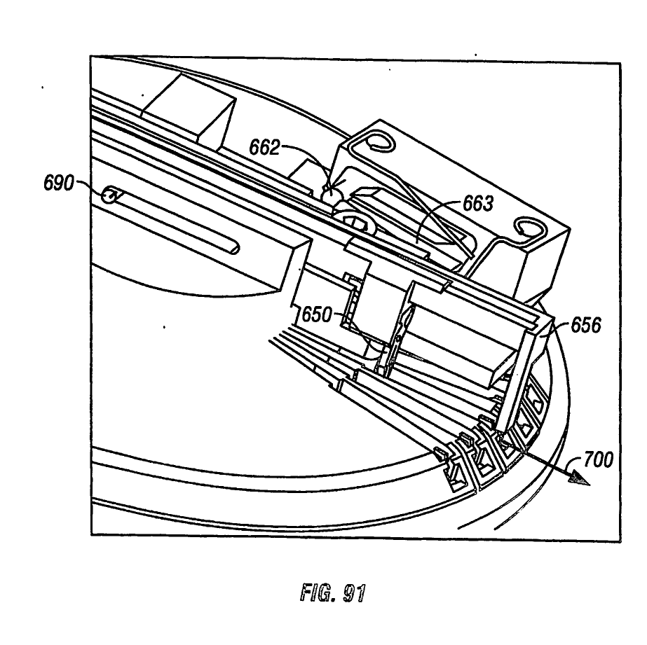

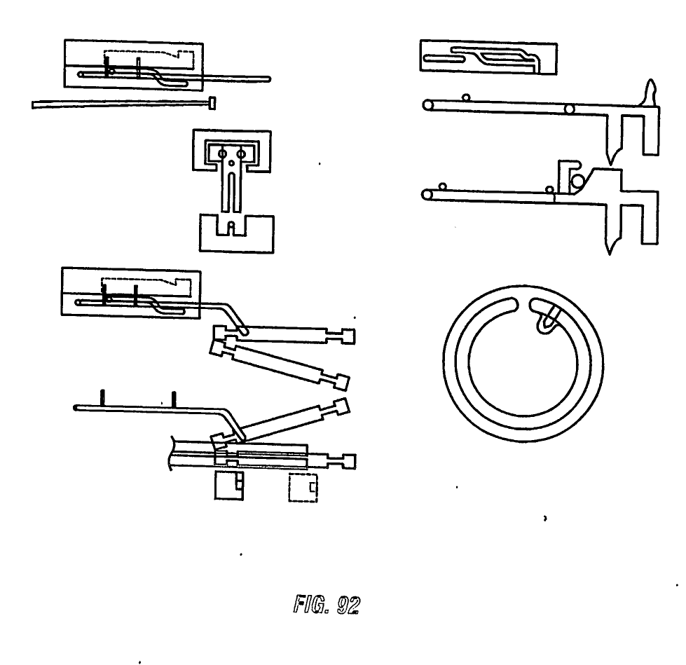

Referring now to the embodiment of FIG. 89, the follower 662 has now moved to the bottom groove 663 and the cutter 680 is moved to an up position. The penetrating member coupler 650 is still in the up position.

FIG. 90 shows the cutter 680 withdrawn, the punch 656 raised and the follower 662 in the bottom groove. In this embodiments, these motions are guided by cam surfaces. The coupler 650 is still raised.

Referring now to FIG. 91, the follower 662 reaches the end of the bottom groove 663. The follower 690 (on pusher 622) pushes down on a cam surface (not shown) that lowers the gripper 650. In this position, the gripper 650 engages a penetrating member in the cavity and can actuate the member outward as indicated by arrow 700.

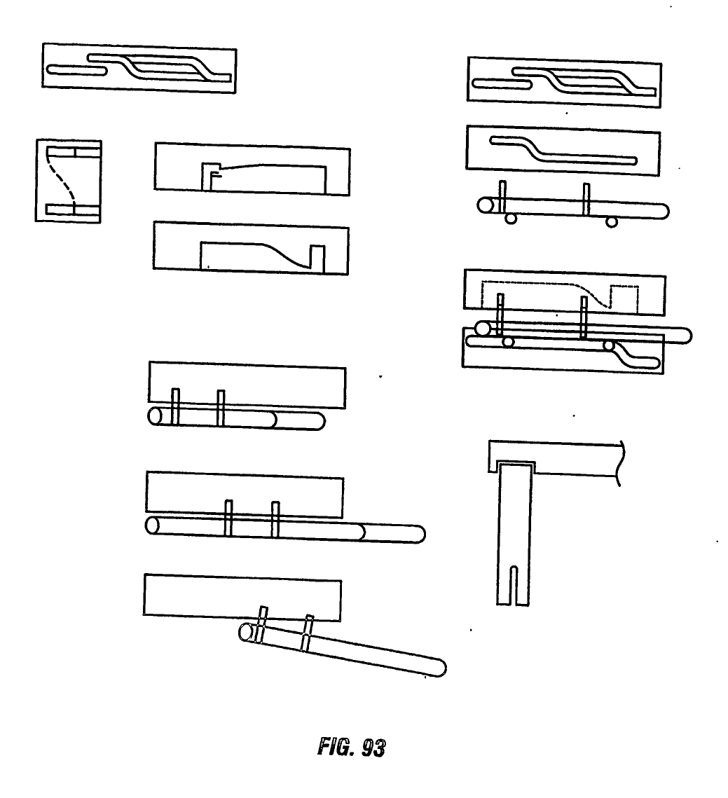

FIGS. 92 and 93 show still further views and embodiments of elements according to the present invention. This embodiment provides a simplified device the both indexes and cuts open the sterility barrier in a single motion. It should be understood that other element besides cams may be used to position the various elements. Rollers, gears, pulleys, electronic, or pneumatic actuators may be used.

Some embodiments may use a pneumatic actuator to advance the penetrating member. The same or a separate pneumatic actuator may be used withdraw the penetrating member. Any other actuator may be used in combination with the pneumatic or other actuator to effect a fast in slow out profile. The devices may be used in place of device 640.

2686

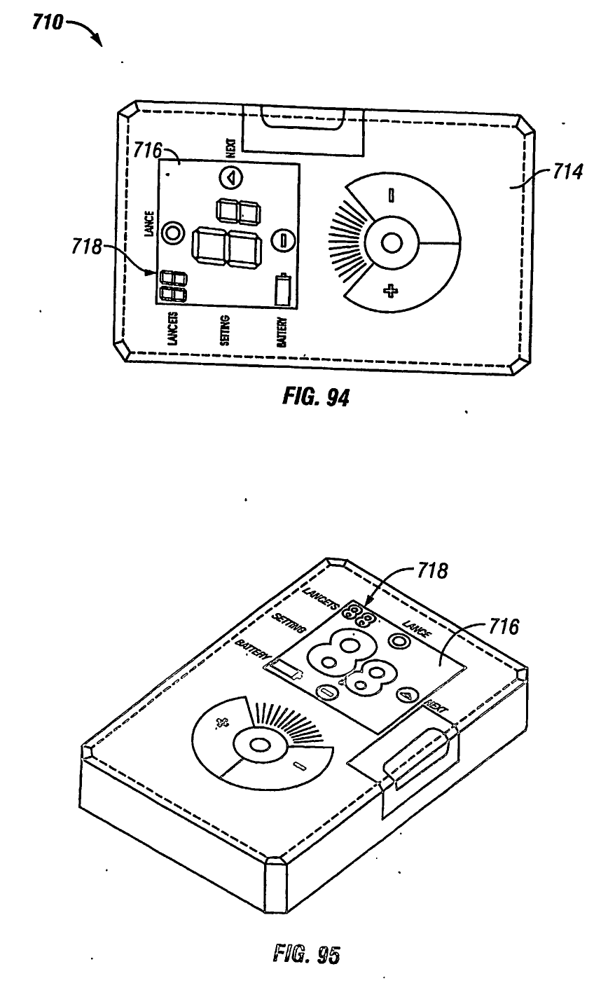

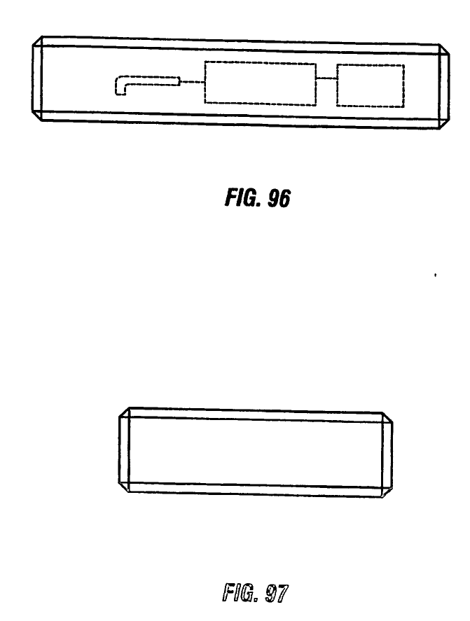

In one embodiment as seen in FIG. 94, a body fluid sampling device 710 for use with a cartridge containing a plurality of penetrating members is provided. The device 710 comprises a penetrating member driver 712 (shown in phantom in FIG. 96) for moving an active one of the penetrating members outward as indicated by arrow 714 from a first position outward to penetrate tissue. A housing 714 is coupled to the driver. A display 716 on the housing shows a number 718 of unused penetrating members remaining. The penetrating member driver is coupled to a position sensor, said sensor used to detect a position of the active one of said penetrating member.

In one embodiment, a body fluid sampling device for use with a cartridge containing a plurality of penetrating members is provided. The device comprises a penetrating member driver for moving an active one of the penetrating members from a first position outward to penetrate tissue. As seen in FIGS. 94 through 97, the housing 714 may have a rectangular configuration.

In one embodiment, a body fluid sampling device for use with a cartridge containing a plurality of penetrating members is provided. The device comprises a penetrating member driver for moving an active one of the penetrating members from a first position outward to penetrate tissue. The housing 714 may have a golden color. As seen in FIG. 96, the position sensor 720 may be used to detect a position of the active one of said penetrating member.

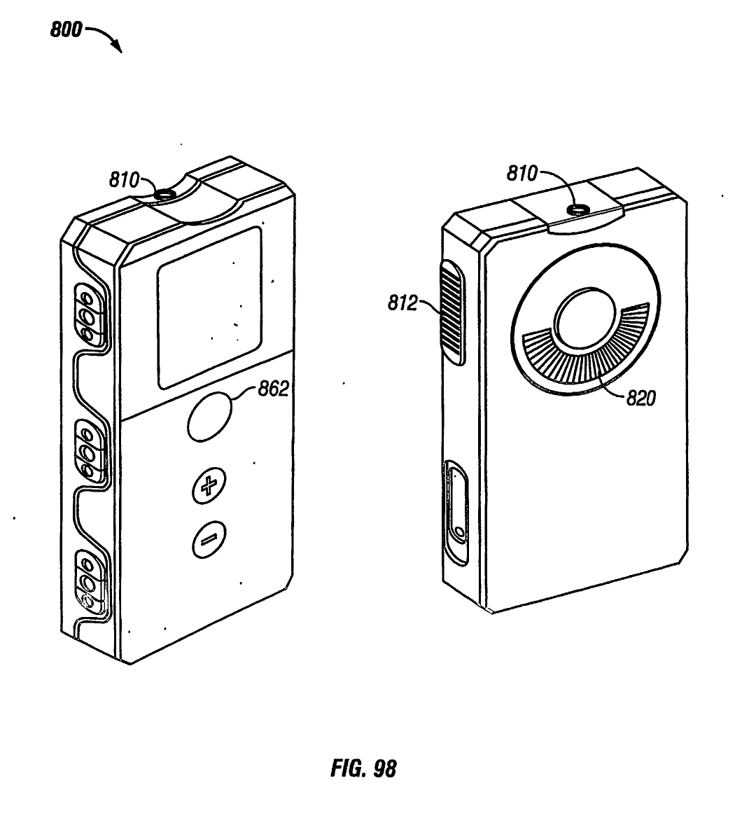

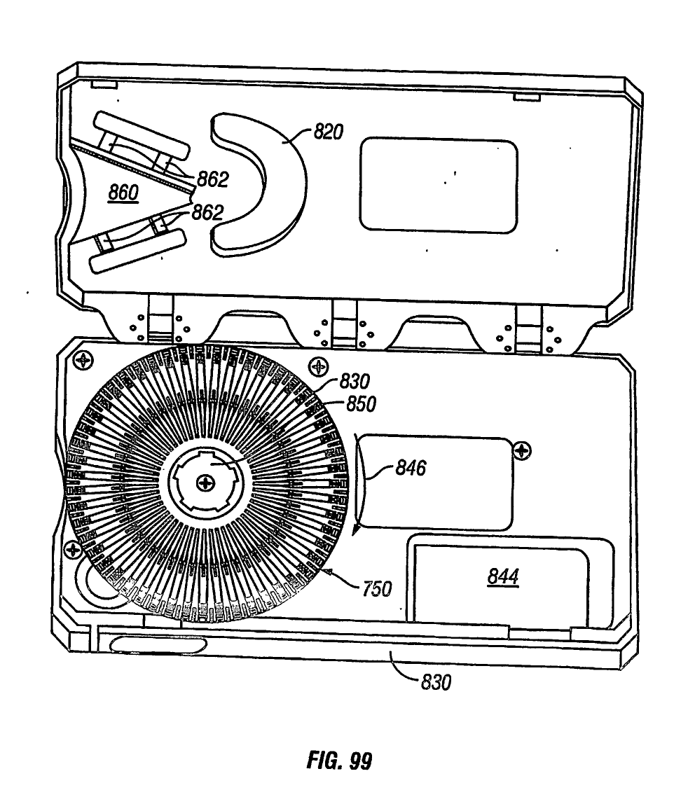

Referring now to FIG. 98, one embodiment of an improved fluid sampling device 800 is shown. The device 800 includes a display 802, a penetrating member actuation button 804, adjustment buttons 806 and 808, and a front end annular ring 810. In this embodiment, a slider 812 is movable as indicated by arrow 814. A pop-open button 816 is provided and is movable as indicated by 818. This opens the device 800 as shown in FIG. 99. This embodiment of device 800 may also include a see-through window 820 that allows a user to see a cartridge inside the device 800. It should be understood that this window 820 may be provided in a variety of shapes including the U-shaped configuration shown in FIG. 97, a full circular window, a U-shaped window on the top portion or mirror-imaged upward from the configuration shown in FIG. 97, comprise of a plurality of smaller windows, or otherwise configured or positioned to show a user that a cartridge is inside the device. Referring now to FIG. 98, the metallic quality of the housing 814 may be observed.

Referring now to FIG. 99, in one embodiment, a body fluid sampling device for use with a cartridge containing a plurality of penetrating members is provided. The device comprises a penetrating member driver for moving an active one of the penetrating members from a first position outward to penetrate tissue. A cavity 750 to house the cartridge 830 containing said penetrating members, said cartridge being a circular disc having a fracturable sterility seal covering a top opening and a side opening.

Referring now to FIG. 99, the embodiment of device 800 is shown with the underside 840 hinged open. A battery 844 is shown in the compartment 842. The cartridge 830 is rotatable as indicated by arrow 846. It should be understood, of course, that the cartridge 830 may be designed to rotate in a counterclockwise direction in another embodiment. A rotatable gear 850 that is linked to slider 812, will rotate to rotate the cartridge 830. In the present embodiment, a support member 860 is provided to position the cartridge 830 in the areas where the penetrating member coupler 870 will engage the penetrating members. Although not limited to the following, the support member 860 is mounted on springs 862 which may allow the support to be moved downward and then urged back to its original position.

Referring now to FIG. 99, in one embodiment, a body fluid sampling device for use with a cartridge containing a plurality of penetrating members is provided. The device comprises a penetrating member driver for moving an active one of the penetrating members from a first position outward to penetrate tissue. The device allows for electronic setting of lancing parameters used by said penetrating member driver. A display 102 may be used to show the settings. Touch pads 180 and 182 may be used to receive input from buttons on the housing.

In one embodiment, a body fluid sampling device for use with a cartridge containing a plurality of penetrating members is provided. The device comprises a penetrating member driver for moving an active one of the penetrating members from a first position outward to penetrate tissue. Settings for lancing parameters used by said penetrating member driver remain in memory without battery. As a nonlimiting example, nonvolatile memory may be used to store the settings. As another nonlimiting example, an EEPROM may be used.

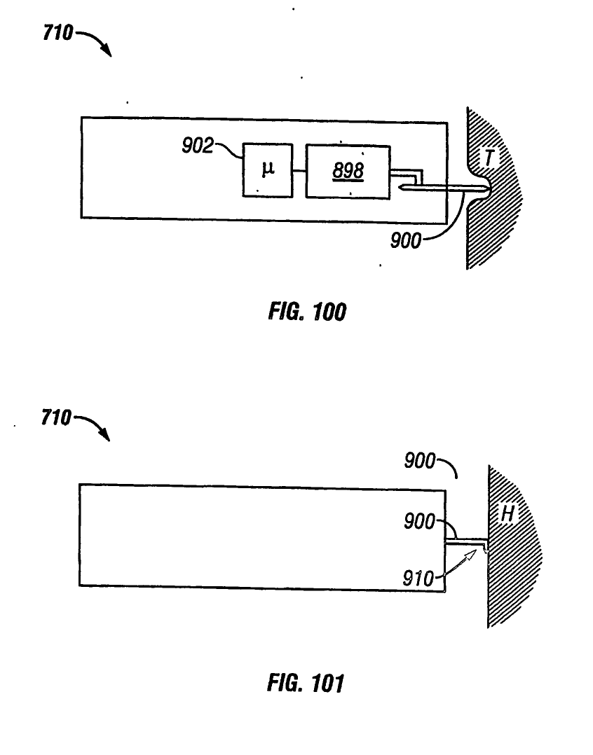

Referring now to FIG. 100, in one embodiment, a body fluid sampling device 710 for use with a cartridge containing a plurality of penetrating members is provided. The device comprises a penetrating member driver 898 for moving an active one 900 of the penetrating members from a first position outward to penetrate tissue. A processor 902 having a safety feature such that the penetrating member driver only lances material with properties similar to skin. Thus as seen in FIG. 100, if the penetrating member 900 in this embodiment fails to decelerate or indicate resistance consistent with that of flesh or skin, the processor 902 will abort the lancing event.

In one embodiment, a body fluid sampling device for use with a cartridge containing a plurality of penetrating members is provided. The device comprises a penetrating member driver for moving an active one of the penetrating members from a first position outward to penetrate tissue. A processor 902 having a safety feature such that the penetrating member driver does not fire the active penetrating member in to air or materials harder than flesh. Thus if the penetrating member fails to sense resistance within a minimum distance such as about 100 microns, 75 microns, 50 microns, or other distance outward from the front end, then the controller will abort lancing event. Of course, other distances may be selected to be the stroke distance before the lancing event is aborted.

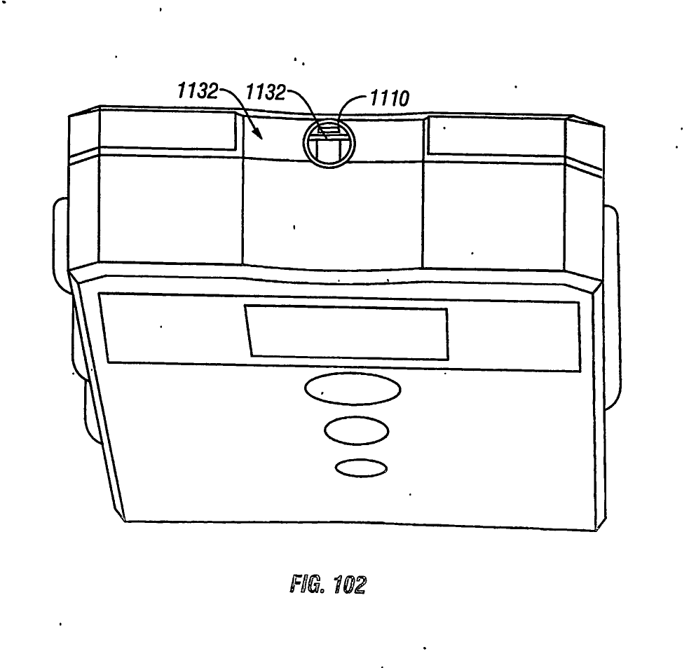

Referring now to FIG. 101, in one embodiment, a body fluid sampling device for use with a cartridge containing a plurality of penetrating members is provided. The device comprises a penetrating member driver for moving an active one of the penetrating members from a first position outward to penetrate tissue. A processor 902 may have a safety feature such that the penetrating member driver wherein a hard detect or an impact against material harder than tissue invalidates usage of the penetrating member 900. As seen in FIG. 100, impact with a hard surface H may cause a spur 910 to form on the distal tip of the penetrating member. Such a penetrating member with a spur 910 or bent tip should not be used.

In one embodiment, a body fluid sampling device for use with a cartridge containing a plurality of penetrating members is provided. The device comprises a penetrating member driver for moving an active one of the penetrating members from a first position outward to penetrate tissue. A display showing penetrating members left/penetrating members spent. A display such as that in FIG. 94 could adapted for such use.

In one embodiment, a body fluid sampling device for use with a cartridge containing a plurality of penetrating members is provided. The device comprises a penetrating member driver for moving an active one of the penetrating members from a first position outward to penetrate tissue. Depth setting of penetrating member penetration into tissue independent of front end geometry. The device of FIG. 94 could be adapted for such use with the buttons on it for adjustments.

In one embodiment, a body fluid sampling device for use with a cartridge containing a plurality of penetrating members is provided. The device comprises a penetrating member driver for moving an active one of the penetrating members from a first position outward to penetrate tissue. The device may include a processor programmed to track position and energy used by the driver to sense position or proximity of skin. Similar to the scheme shown in FIG. 8, if the penetrating member fails to engage tissue or material with tissue like resistance, lancing event is aborted.

In one embodiment, a body fluid sampling device for use with a cartridge containing a plurality of penetrating members is provided. The device comprises a penetrating member driver for moving an active one of the penetrating members from a first position outward to penetrate tissue. A display that has a screen saver may be used with the device. The device of FIG. 94 could be adapted for such use. As a nonlimiting example, the screensaver may be a small circle moving on the screen, a pattern being repeated on the display, or as other screen savers known to those of skill in the art.

In one embodiment, a body fluid sampling device for use with a cartridge containing a plurality of penetrating members is provided. The device comprises a penetrating member driver for moving an active one of the penetrating members from a first position outward to penetrate tissue. A display may be provided that relays a “too deep” signal to a user based on the lancing event. The device of FIG. 1 could be adapted for such use. Thus a processor 902 may determine that based on the resistance or other factors, the penetrating member went too deep. In some embodiments, this may also be based on whether the user indicated if blood was spontaneously generated, degree of pain felt, or other feedback. In other embodiments, the too deep indicator is based purely on tissue qualities detected by device and fed to the processor.

In one embodiment, the lancing device is the first fully-automated and integrated lancing system. Simply hold it up to the skin and press of a button. Instantly and painlessly a blood sample will appear. The device dramatically reduces the pain and inconvenience of other lancing systems. With a self-contained, sterile 50 lancet disk and fast, automatically controlled lancing a patient never needs to handle a new or used lancet again. They experience less pain and benefit from faster wound healing.

In one embodiment, the lancing device is based on the “one-step, one-button” approach. Simply hold the device up to the skin and press the button. Instantly, a painless blood sample will appear. (One can milk if more blood is needed.)

The lancing device incorporates a self-contained lancing disk that eliminates the need to load and discard lancets. The 50-lancet disk simply needs to be replaced after 50 uses.

As the first and only fully automated, self-contained lancing device, in one embodiment, the lancing device offers advantages over other lancing systems in 5 main areas: Pain, Convenience, Safety, Wound Healing, and Reliability.

1. Pain: Smart Lancing technology allows for fast, yet smooth electronically controlled lancing. Additionally, the lancing device is able to assess an individual's skin composition and only lance to lowest possible depth necessary to obtain an adequate sample.

2. & 3. Convenience and Safety: The device is completely self-contained and electronically controlled. The device only requires a user to press a single button to execute the entire lancing process. The device incorporates a self-contained, sterile 50-lancet disk so individuals with diabetes, most importantly children, will never see or handle a new or used lancet ever again.

4. Wound Healing: The fast, yet smooth electronically controlled lancing allows for minimal tissue damage and faster wound healing.

5. Reliability: The device eliminates the need for repetitive lancing due to insufficient blood samples. The lancing device results in an adequate blood sample nearly every single time.

We recommend using the disk to completion. In one embodiment, however, a partially used Disk can be used. Once re-inserted the number of lancets remaining will be displayed on the screen and the device will be ready for use.

In one embodiment, the lancing device is designed for the more convenient and physiologically accurate fingertip testing. Because the lancing device offers dramatically reduced pain and faster wound healing, AST is no longer necessary for patients.

The lancing device is designed for use by all individuals with diabetes and will be marketed to all individuals with diabetes who test their blood glucose levels. However, the lancing device is especially beneficial to children, who have sensitive skin and benefit the most from integrated convenience, safety, reduced pain, and faster wound healing.

In one embodiment, the product is a “one-step, one button” fully integrated blood glucose sampling and measurement solution. This product combines the sample acquisition benefits of the lancing device with superior measurement technology in one convenient device that will significantly reduce pain, eliminate daily medical waste, and provide superior monitoring reliability.

Like the lancing device, the second-generation glucose sampling and measurement device is completely self-contained and fully automated. This will allow a patient to execute the entire sample acquisition and measurement process by pressing one button. Patients will no longer need to have separate lancing devices and glucose meters. This product will be the only device that individuals with diabetes will need for glucose monitoring activities.

In addition to the reduced pain, increased convenience and enhanced reliability of the lancing device, this second-generation device also incorporates a replaceable combined test-strip/lancet disk that eliminates the need for patients to load and discard lancets or test strips.

A plurality of conventional fluid sampling and specifically, glucose spot monitoring devices are shown. As a nonlimiting example, the one step solution of the present invention may combine the steps 1-7 into a single process to the user. In one embodiment, this may comprise pressing a single button to lance and obtain a blood glucose or other analyte reading. In other embodiments, the one step may be augmented by a step using a slider that is movable to load a new unused penetrating member in active position. In some embodiments, this may involve rotating the cartridge to bring the member into alignment with the penetrating member coupler.

The evolution of integrated fluid sampling and monitoring devices will now be described. As seen, adaptive control may be added to a fluid sampling device to provide improved control of penetrating member depth. Adaptive control may involve an algorithm in the device controller which may take input from the user such as but not limited whether blood was spontaneously generated, pain level, sufficient blood generation, time of day during lancing, hydration of the user, location selected for lancing, or like from a lancing event. The algorithm will take these at least one of these variables and adjust the next lancing event to optimize lancing performance. Adjustments may include, but are not limited to, increasing or decreasing desired depth, adjusting velocity profile, changing braking force, changing coasting time, adjusting for tenting of tissue, or the like. It should be understood that other adjustments to penetrating member performance may be made to improve spontaneous blood generation or to reduce user pain.

One embodiment of an improved fluid sampling device 1100 is shown. The device 1100 includes a display 1102, a penetrating member actuation button 1104, adjustment buttons 1106 and 1108, and a front end annular ring 1110. In this embodiment, a slider 1112 is movable as indicated by arrow 1114. A pop-open button 1116 is provided and is movable as indicated by 1118. This opens the device 1100 as shown in FIG. 103. This embodiment of device 1100 may also include a see-through window 1120 that allows a user to see a cartridge inside the device 1100. It should be understood that this window 1120 may be provided in a variety of shapes including the U-shaped configuration shown in FIG. 97, a full circular window, a U-shaped window on the top portion or mirror-imaged upward from the configuration, comprise of a plurality of smaller windows, or otherwise configured or positioned to show a user that a cartridge is inside the device.

Referring now to FIG. 102, close-up view is shown of the front end annular ring 1110. The cartridge 1130 with a sterility barrier can be seen through the front end. It should be understood that in some embodiments, the portion 1132 of the housing having the front end annular ring 1110 may be removed to allow the entire annular ring 1110 to be replaced either for cleaning purposes or for removal in its entirety.

Referring now to FIG. 103, a portion of the interior of the device 1100 is shown with the bottom portion 1140 being opened. A battery compartment 1142 is provided to house a power source for this embodiment of the device 1100.

The embodiment of device 1100 is shown with the underside 1140 hinged open. A battery 1144 is shown in the compartment 1142. The cartridge 1130 is rotatable as indicated by arrow 1146. It should be understood, of course, that the cartridge 1130 may be designed to rotate in a counterclockwise direction in another embodiment. A rotatable gear 1150 (shown more clearly in FIG. 106) that is linked to slider 1112, will rotate to rotate the cartridge 1130. In the present embodiment, a support member 1160 is provided to position the cartridge 1130 in the areas where the penetrating member coupler 1170 will engage the penetrating members. Although not limited to the following, the support member 1160 is mounted on springs 1162 which may allow the support to be moved downward and then urged back to its original position.

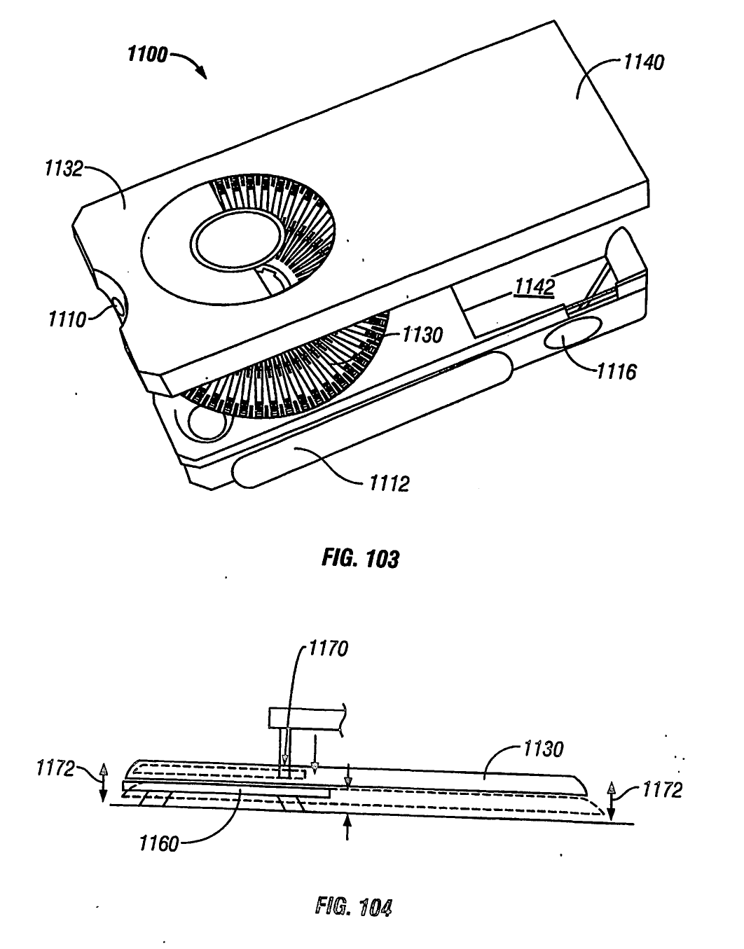

In this embodiment as shown in FIG. 104, this allows the penetrating member coupler 1170 to move downward in a longer stroke. The stroke length helps to ensure that the coupler 1170 engages the penetrating member in the cartridge 1130. The support member 1160 and the entire cartridge 1130 may be moved downward and upward as indicated by arrows 1172. The maximum downward position of the cartridge 1130 and the support member 1160 is shown in phantom. In the present embodiment, the support member 1160 is positioned to not interfere with the rotation of the cartridge 1130 but allows the cartridge 1130 be sufficient supported and positioned in place to allow a penetrating member coupler to accurately engage the cartridge 1130 and a penetrating member held therein.

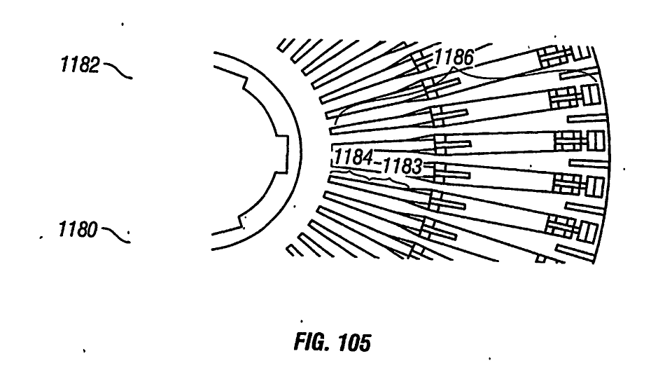

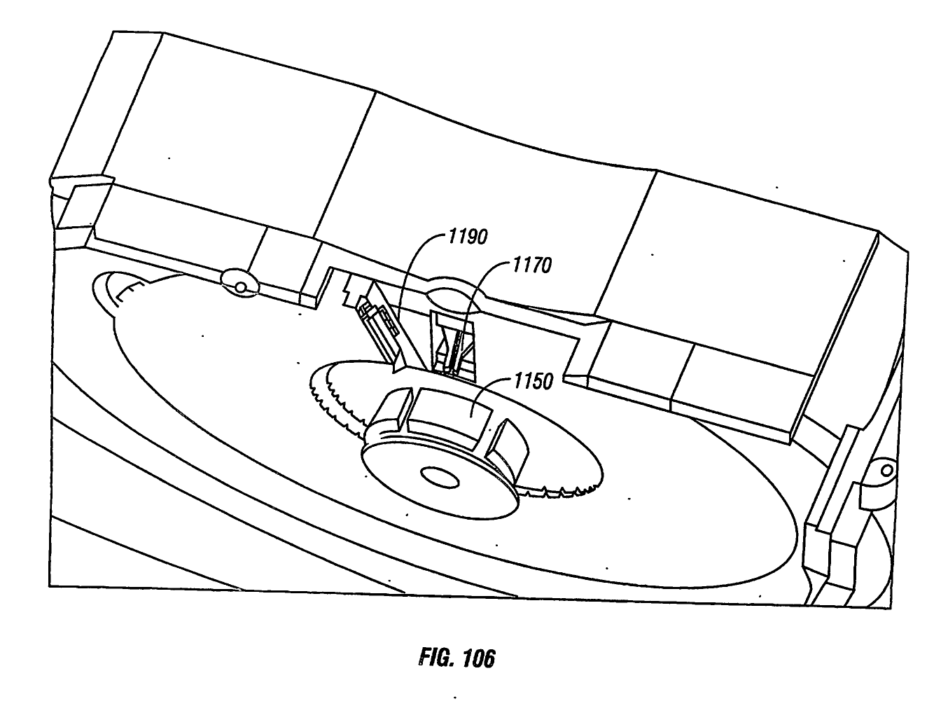

Referring now to FIG. 105, a close-up top down view is shown of an embodiment of a cartridge 1130 according to the present invention. As seen in FIG. 104, the inner circumference may include a notch 1180 to facilitate positioning and/or rotation of the cartridge 1130. The cartridge 1130 may also include gear teeth 1182 to engage a corresponding gear on the gear 1150 shown in FIG. 106. As seen in the FIG. 104, the cartridge 1130 may include a park portion 1184 to hold a penetrating member 1186 in the cartridge. The rear bearing 1188 will, in this embodiment, remain in guide contact with the penetrating member 1186 so that the penetrating member does not stray while it is being actuated.

Referring now to FIG. 106, a close-up view is shown of the components used to engage and actuate a penetrating member. A penetrating member coupler 1170 is shown along with a punch 1190 used to open a sterility barrier covering the cartridge 1130 and keeping the penetrating member in a sterile condition prior to use. Further details on the function of the coupler 1170 and punch 1190 are discussed in U.S. patent application Attorney Docket No. 38187-2606. Gear 1150 will rotate to increment the cartridge 1130 as desired. Movement of the gear 1150 may be coupled to a slider 1112.

It may not take much time to obtain a fluid sample and provide a reading. In one embodiment of the present invention, the device may provide a time of less than about 20 seconds using an electronic lancing device. The method may also be one that uses actuation based on a single button. Improved embodiments such as GM1 and GM2 as seen in U.S. patent application Attorney Docket No. 38187-2662 fully incorporated herein by reference may provide total times of less than about 15 seconds and less than about 10 seconds.

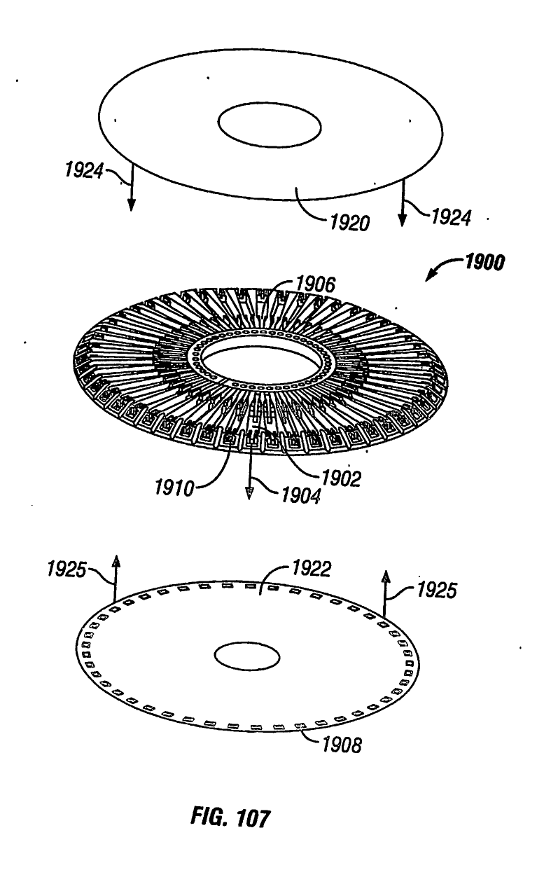

Referring now to FIG. 107, a still further embodiment of a cartridge according to the present invention will be described. FIG. 107 shows one embodiment of a cartridge 900 which may be removably inserted into an apparatus for driving penetrating members to pierce skin or tissue. The cartridge 900 has a plurality of penetrating members 902 that may be individually or otherwise selectively actuated so that the penetrating members 902 may extend outward from the cartridge, as indicated by arrow 904, to penetrate tissue. In the present embodiment, the cartridge 900 may be based on a flat disc with a number of penetrating members such as, but in no way limited to, (25, 50, 75, 100, . . . ) arranged radially on the disc or cartridge 800. It should be understood that although the cartridge 900 is shown as a disc or a disc-shaped housing, other shapes or configurations of the cartridge may also work without departing from the spirit of the present invention of placing a plurality of penetrating members to be engaged, singly or in some combination, by a penetrating member driver.

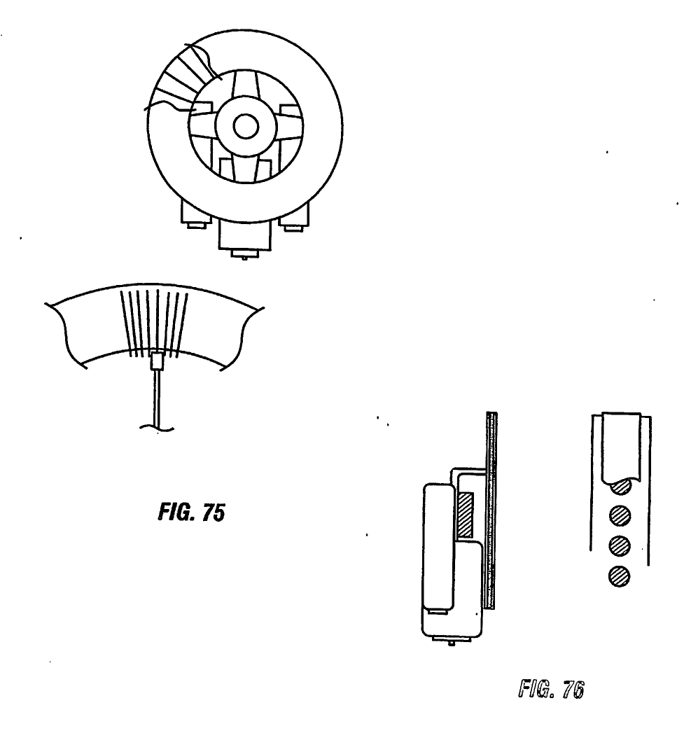

Each penetrating member 902 may be contained in a cavity 906 in the cartridge 900 with the penetrating member's sharpened end facing radially outward and may be in the same plane as that of the cartridge. The cavity 906 may be molded, pressed, forged, or otherwise formed in the cartridge. Although not limited in this manner, the ends of the cavities 906 may be divided into individual fingers (such as one for each cavity) on the outer periphery of the disc. The particular shape of each cavity 906 may be designed to suit the size or shape of the penetrating member therein or the amount of space desired for placement of the analyte detecting members 808. For example and not limitation, the cavity 906 may have a V-shaped cross-section, a U-shaped cross-section, C-shaped cross-section, a multi-level cross section or the other cross-sections. The opening 810 through which a penetrating member 902 may exit to penetrate tissue may also have a variety of shapes, such as but not limited to, a circular opening, a square or rectangular opening, a U-shaped opening, a narrow opening that only allows the penetrating member to pass, an opening with more clearance on the sides, a slit, a configuration as shown in FIG. 75, or the other shapes.

In this embodiment, after actuation, the penetrating member 902 is returned into the cartridge and may be held within the cartridge 900 in a manner so that it is not able to be used again. By way of example and not limitation, a used penetrating member may be returned into the cartridge and held by the launcher in position until the next lancing event. At the time of the next lancing, the launcher may disengage the used penetrating member with the cartridge 900 turned or indexed to the next clean penetrating member such that the cavity holding the used penetrating member is position so that it is not accessible to the user (i.e. turn away from a penetrating member exit opening). In some embodiments, the tip of a used penetrating member may be driven into a protective stop that hold the penetrating member in place after use. The cartridge 900 is replaceable with a new cartridge 900 once all the penetrating members have been used or at such other time or condition as deemed desirable by the user.

Referring still to the embodiment in FIG. 107, the cartridge 900 may provide sterile environments for penetrating members via seals, foils, covers, polymeric, or similar materials used to seal the cavities and provide enclosed areas for the penetrating members to rest in. In the present embodiment, a foil seal layer 920 is applied to one surface of the cartridge 900. The seal layer 920 may be made of a variety of materials such as a metallic foil or other seal materials and may be of a tensile strength and other quality that may provide a sealed, sterile environment until the seal layer 920 is penetrate by a suitable or penetrating device providing a preselected or selected amount of force to open the sealed, sterile environment. Each cavity 906 may be individually sealed with a layer 920 in a manner such that the opening of one cavity does not interfere with the sterility in an adjacent or other cavity in the cartridge 800. As seen in the embodiment of FIG. 107, the seal layer 920 may be a planar material that is adhered to a top surface of the cartridge 800.

Depending on the orientation of the cartridge 900 in the penetrating member driver apparatus, the seal layer 920 may be on the top surface, side surface, bottom surface, or other positioned surface. For ease of illustration and discussion of the embodiment of FIG. 107, the layer 920 is placed on a top surface of the cartridge 800. The cavities 906 holding the penetrating members 902 are sealed on by the foil layer 920 and thus create the sterile environments for the penetrating members. The foil layer 920 may seal a plurality of cavities 906 or only a select number of cavities as desired.

In a still further feature of FIG. 107, the cartridge 900 may optionally include a plurality of analyte detecting members 908 on a substrate 922 which may be attached to a bottom surface of the cartridge 900. The substrate may be made of a material such as, but not limited to, a polymer, a foil, or other material suitable for attaching to a cartridge and holding the analyte detecting members 908. As seen in FIG. 107, the substrate 922 may hold a plurality of analyte detecting members, such as but not limited to, about 10-50, 50-100, or other combinations of analyte detecting members. This facilitates the assembly and integration of analyte detecting members 908 with cartridge 900. These analyte detecting members 908 may enable an integrated body fluid sampling system where the penetrating members 902 create a wound tract in a target tissue, which expresses body fluid that flows into the cartridge for analyte detection by at least one of the analyte detecting members 908. The substrate 922 may contain any number of analyte detecting members 908 suitable for detecting analytes in cartridge having a plurality of cavities 906. In one embodiment, many analyte detecting members 908 may be printed onto a single substrate 922 which is then adhered to the cartridge to facilitate manufacturing and simplify assembly. The analyte detecting members 908 may be electrochemical in nature. The analyte detecting members 908 may further contain enzymes, dyes, or other detectors which react when exposed to the desired analyte. Additionally, the analyte detecting members 908 may comprise of clear optical windows that allow light to pass into the body fluid for analyte analysis. The number, location, and type of analyte detecting member 908 may be varied as desired, based in part on the design of the cartridge, number of analytes to be measured, the need for analyte detecting member calibration, and the sensitivity of the analyte detecting members. If the cartridge 900 uses an analyte detecting member arrangement where the analyte detecting members are on a substrate attached to the bottom of the cartridge, there may be through holes (as shown in FIG. 76), wicking elements, capillary tube or other devices on the cartridge 900 to allow body fluid to flow from the cartridge to the analyte detecting members 908 for analysis. In other configurations, the analyte detecting members 908 may be printed, formed, or otherwise located directly in the cavities housing the penetrating members 902 or areas on the cartridge surface that receive blood after lancing.

The use of the seal layer 920 and substrate or analyte detecting member layer 822 may facilitate the manufacture of these cartridges 10. For example, a single seal layer 920 may be adhered, attached, or otherwise coupled to the cartridge 900 as indicated by arrows 924 to seal many of the cavities 906 at one time. A sheet 922 of analyte detecting members may also be adhered, attached, or otherwise coupled to the cartridge 900 as indicated by arrows 925 to provide many analyte detecting members on the cartridge at one time. During manufacturing of one embodiment of the present invention, the cartridge 900 may be loaded with penetrating members 902, sealed with layer 920 and a temporary layer (not shown) on the bottom where substrate 922 would later go, to provide a sealed environment for the penetrating members. This assembly with the temporary bottom layer is then taken to be sterilized. After sterilization, the assembly is taken to a clean room (or it may already be in a clear room or equivalent environment) where the temporary bottom layer is removed and the substrate 922, with analyte detecting members is coupled to the cartridge as shown in FIG. 107. This process allows for the sterile assembly of the cartridge with the penetrating members 902 using processes and/or temperatures that may degrade the accuracy or functionality of the analyte detecting members on substrate 922. As a nonlimiting example, the entire cartridge 900 may then be placed in a further sealed container such as a pouch, bag, plastic molded container, etc. to facilitate contact, improve ruggedness, and/or allow for easier handling.

In some embodiments, more than one seal layer 920 may be used to seal the cavities 906. As examples of some embodiments, multiple layers may be placed over each cavity 906, half or some selected portion of the cavities may be sealed with one layer with the other half or selected portion of the cavities sealed with another sheet or layer, different shaped cavities may use different seal layer, or the like. The seal layer 920 may have different physical properties, such as those covering the penetrating members 902 near the end of the cartridge may have a different color such as red to indicate to the user (if visually inspectable) that the user is down to say 10, 5, or other number of penetrating members before the cartridge should be changed out.

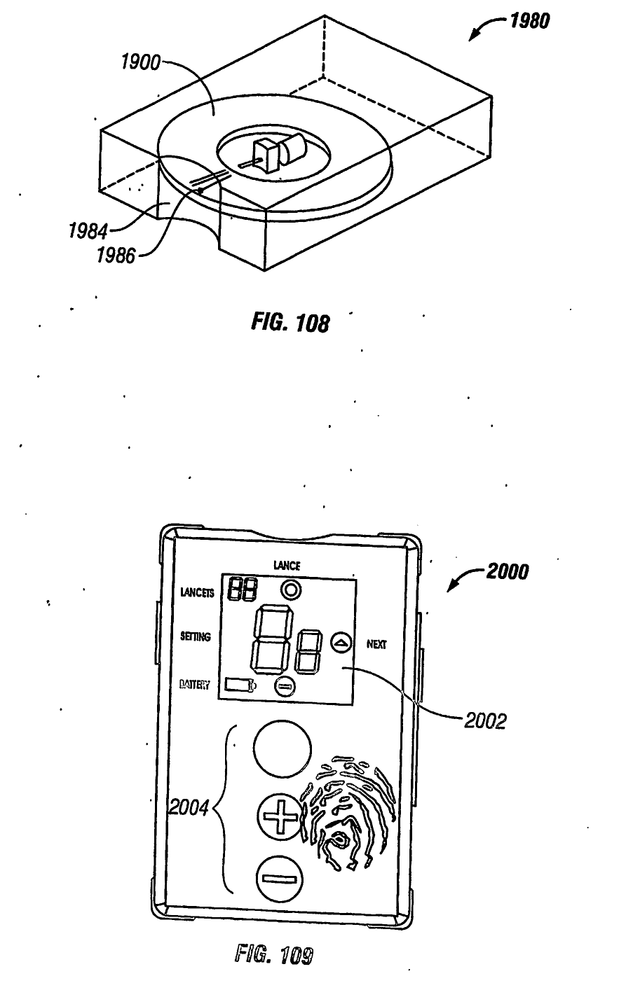

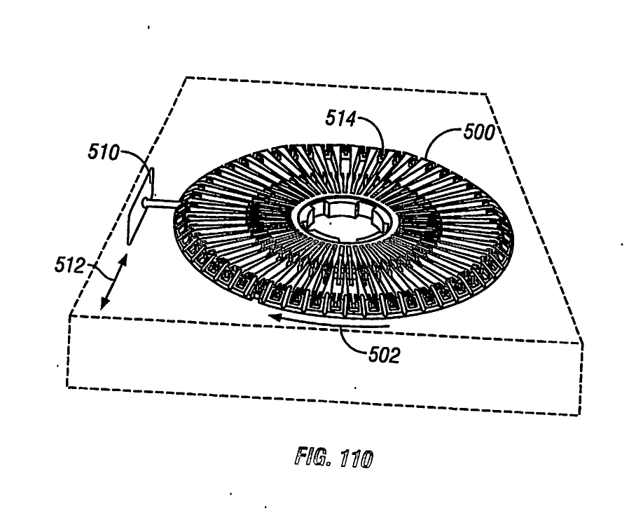

Referring now to FIG. 108, one embodiment of an apparatus 980 using a radial cartridge 900 with a penetrating member driver 982 is shown. A contoured surface 884, is located near a penetrating member exit port 986, allowing for a patient to place their finger in position for lancing. Although not shown, the apparatus 980 may include a human readable or other type of visual display to relay status to the user. The display may also show measured analyte levels or other measurement or feedback to the user without the need to plug apparatus 980 or a separate test strip into a separate analyte reader device. The apparatus 980 may include a processor or other logic for actuating the penetrating member or for measuring the analyte levels. The cartridge 900 may be loaded into the apparatus 980 by opening a top housing of the apparatus which may be hinged or removably coupled to a bottom housing. The cartridge 900 may also drawn into the apparatus 980 using a loading mechanism similar in spirit to that found on a compact disc player or the like. In such an embodiment, the apparatus may have a slot (similar to a CD player in an automobile) that allows for the insertion of the cartridge 900 into the apparatus 980 which is then automatically loaded into position or otherwise seated in the apparatus for operation therein. The loading mechanism may be mechanically powered or electrically powered. In some embodiments, the loading mechanism may use a loading tray in addition to the slot. The slot may be placed higher on the housing so that the cartridge 900 will have enough clearance to be loaded into the device and then dropped down over the penetrating member driver 982. The cartridge 900 may have an indicator mark or indexing device that allows the cartridge to be properly aligned by the loading mechanism or an aligning mechanism once the cartridge 900 is placed into the apparatus 980. The cartridge 900 may rest on a radial platform that rotates about the penetrating member driver 982, thus providing a method for advancing the cartridge to bring unused penetrating members to engagement with the penetrating member driver. The cartridge 800 on its underside or other surface, may shaped or contoured such as with notches, grooves, tractor holes, optical markers, or the like to facilitate handling and/or indexing of the cartridge. These shapes or surfaces may also be varied so as to indicate that the cartridge is almost out of unused penetrating members, that there are only five penetrating members left, or some other cartridge status indicator as desired.

A suitable method and apparatus for loading penetrating members has been described previously in commonly assigned, copending U.S. patent applications Attorney Docket 38187-2589 and 38187-2590, and are included here by reference for all purposes. Suitable devices for engaging the penetrating members and for removing protective materials associated with the penetrating member cavity are described in commonly assigned, copending U.S. patent applications Attorney Docket 38187-2601 and 38187-2602, and are included here by reference for all purposes. For example in the embodiment of FIG. 107, the foil or seal layer 920 may cover the cavity by extending across the cavity along a top surface 990 and down along the angled surface 892 to provide a sealed, sterile environment for the penetrating member and sensors therein. A piercing element described in U.S. patent applications Attorney Docket 38187-2602 has a piercing element and then a shaped portion behind the element which pushes the foil to the sides of the cavity or other position so that the penetrating member 902 may be actuated and body fluid may flow into the cavity.

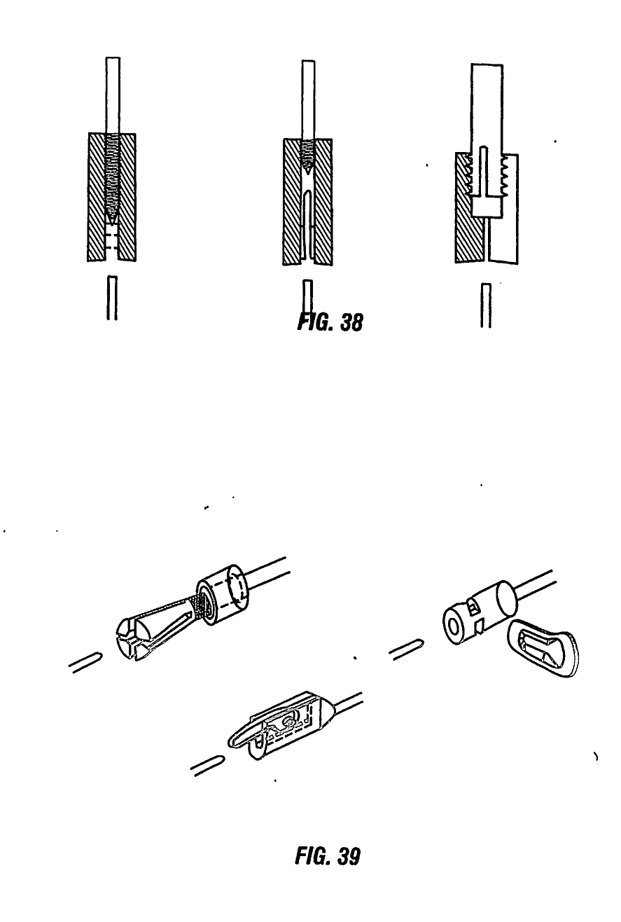

Referring now to FIG. 39, one embodiment of a device that may use a disc 900 is shown. This embodiment of device 1000 include a display 1002 that shows lancing performance and setting such as penetration depth setting the like. Various buttons 1004 may also be placed on the housing to adjust settings and/or to activate lancing.

It should be understood that device 1000 may include a processor for implementing any of the control methodologies set forth herein. The processor may control the penetrating member driver and/or active braking device such a pads, stops, dampers, dashpots and other mechanism to control penetrating member speed. The characteristic phases of penetrating member advancement and retraction can be plotted on a graph of force versus time illustrating the force exerted by the penetrating member driver on the penetrating member to achieve the desired displacement and velocity profile. The characteristic phases are the penetrating member introduction phase A-C where the penetrating member is longitudinally advanced into the skin, the penetrating member rest phase D where the penetrating member terminates its longitudinal movement reaching its maximum depth and becoming relatively stationary, and the penetrating member retraction phase E-G where the penetrating member is longitudinally retracted out of the skin. The duration of the penetrating member retraction phase E-G is longer than the duration of the penetrating member introduction phase A-C, which in turn is longer than the duration of the penetrating member rest phase D.

The introduction phase further comprises a penetrating member launch phase prior to A when the penetrating member is longitudinally moving through air toward the skin, a tissue contact phase at the beginning of A when the distal end of the penetrating member makes initial contact with the skin, a tissue deformation phase A when the skin bends depending on its elastic properties which are related to hydration and thickness, a tissue lancing phase which comprises when the penetrating member hits the inflection point on the skin and begins to cut the skin B and the penetrating member continues cutting the skin C. The penetrating member rest phase D is the limit of the penetration of the penetrating member into the skin. Pain is reduced by minimizing the duration of the penetrating member introduction phase A-C so that there is a fast incision to a certain penetration depth regardless of the duration of the deformation phase A and inflection point cutting B which will vary from user to user. Success rate is increased by measuring the exact depth of penetration from inflection point B to the limit of penetration in the penetrating member rest phase D. This measurement allows the penetrating member to always, or at least reliably, hit the capillary beds which are a known distance underneath the surface of the skin.

The penetrating member retraction phase further comprises a primary retraction phase E when the skin pushes the penetrating member out of the wound tract, a secondary retraction phase F when the penetrating member starts to become dislodged and pulls in the opposite direction of the skin, and penetrating member exit phase G when the penetrating member becomes free of the skin. Primary retraction is the result of exerting a decreasing force to pull the penetrating member out of the skin as the penetrating member pulls away from the finger. Secondary retraction is the result of exerting a force in the opposite direction to dislodge the penetrating member. Control is necessary to keep the wound tract open as blood flows up the wound tract. Blood volume is increased by using a uniform velocity to retract the penetrating member during the penetrating member retraction phase E-G regardless of the force required for the primary retraction phase E or secondary retraction phase F, either of which may vary from user to user depending on the properties of the user's skin.

Displacement versus time profile of a penetrating member for a controlled penetrating member retraction can be plotted. Velocity vs. time profile of the penetrating member for the controlled retraction cann also be plotted. The penetrating member driver controls penetrating member displacement and velocity at several steps in the lancing cycle, including when the penetrating member cuts the blood vessels to allow blood to pool 2130, and as the penetrating member retracts, regulating the retraction rate to allow the blood to flood the wound tract while keeping the wound flap from sealing the channel 2132 to permit blood to exit the wound.

The tenting process and retrograde motion of the penetrating member during the lancing cycle can be illustrated graphically which shows both a velocity versus time graph and a position versus time graph of a penetrating member tip during a lancing cycle that includes elastic and inelastic tenting. From point 0 to point A, the penetrating member is being accelerated from the initialization position or zero position. From point A to point B, the penetrating member is in ballistic or coasting mode, with no additional power being delivered. At point B, the penetrating member tip contacts the tissue and begins to tent the skin until it reaches a displacement C. As the penetrating member tip approaches maximum displacement, braking force is applied to the penetrating member until the penetrating member comes to a stop at point D. The penetrating member then recoils in a retrograde direction during the settling phase of the lancing cycle indicated between D and E. Note that the magnitude of inelastic tenting indicated in FIG. 148 is exaggerated for purposes of illustration.

The amount of inelastic tenting indicated by Z tends to be fairly consistent and small compared to the magnitude of the elastic tenting. Generally, the amount of inelastic tenting Z can be about 120 to about 140 microns. As the magnitude of the inelastic tenting has a fairly constant value and is small compared to the magnitude of the elastic tenting for most patients and skin types, the value for the total amount of tenting for the penetration stroke of the penetrating member is effectively equal to the rearward displacement of the penetrating member during the settling phase as measured by the processor 193 plus a predetermined value for the inelastic recoil, such as 130 microns. Inelastic recoil for some embodiments can be about 100 to about 200 microns. The ability to measure the magnitude of skin tenting for a patient is important to controlling the depth of penetration of the penetrating member tip as the skin is generally known to vary in elasticity and other parameters due to age, time of day, level of hydration, gender and pathological state.

This value for total tenting for the lancing cycle can then be used to determine the various characteristics of the patient's skin. Once a body of tenting data is obtained for a given patient, this data can be analyzed in order to predict the total penetrating member displacement, from the point of skin contact, necessary for a successful lancing procedure. This enables the tissue penetration device to achieve a high success rate and minimize pain for the user. A rolling average table can be used to collect and store the tenting data for a patient with a pointer to the last entry in the table. When a new entry is input, it can replace the entry at the pointer and the pointer advances to the next value. When an average is desired, all the values are added and the sum divided by the total number of entries by the processor 193. Similar techniques involving exponential decay (multiply by 0.95, add 0.05 times current value, etc.) are also possible.

With regard to tenting of skin generally, some typical values relating to penetration depth are now discussed. A cross sectional view of the layers of the skin can be shown. In order to reliably obtain a useable sample of blood from the skin, it is desirable to have the penetrating member tip reach the venuolar plexus of the skin. The stratum corneum is typically about 0.1 to about 0.6 mm thick and the distance from the top of the dermis to the venuole plexus can be from about 0.3 to about 1.4 mm. Elastic tenting can have a magnitude of up to about 2 mm or so, specifially, about 0.2 to about 2.0 mm, with an average magnitude of about 1 mm. This means that the amount of penetrating member displacement necessary to overcome the tenting can have a magnitude greater than the thickness of skin necessary to penetrate in order to reach the venuolar plexus. The total penetrating member displacement from point of initial skin contact may have an average value of about 1.7 to about 2.1 mm. In some embodiments, penetration depth and maximum penetration depth may be about 0.5 mm to about 5 mm, specifically, about 1 mm to about 3 mm. In some embodiments, a maximum penetration depth of about 0.5 to about 3 mm is useful.

In some embodiments, the penetrating member is withdrawn with less force and a lower speed than the force and speed during the penetration portion of the operation cycle. Withdrawal speed of the penetrating member in some embodiments can be about 0.004 to about 0.5 m/s, specifically, about 0.006 to about 0.01 m/s. In other embodiments, useful withdrawal velocities can be about 0.001 to about 0.02 meters per second, specifically, about 0.001 to about 0.01 meters per second. For embodiments that use a relatively slow withdrawal velocity compared to the penetration velocity, the withdrawal velocity may up to about 0.02 meters per second. For such embodiments, a ratio of the average penetration velocity relative to the average withdrawal velocity can be about 100 to about 1000. In embodiments where a relatively slow withdrawal velocity is not important, a withdrawal velocity of about 2 to about 10 meters per second may be used.

Another example of an embodiment of a velocity profile for a penetrating member can be seen shown, which illustrates a penetrating member profile with a fast entry velocity and a slow withdrawal velocity. A lancing profile showing velocity of the penetrating member versus position. The lancing profile starts at zero time and position and shows acceleration of the penetrating member towards the tissue from the electromagnetic force generated from the electromagnetic driver. At point A, the power is shut off and the penetrating member begins to coast until it reaches the skin indicated by B at which point, the velocity begins to decrease. At point C, the penetrating member has reached maximum displacement and settles momentarily, typically for a time of about 8 milliseconds. %

A retrograde withdrawal force is then imposed on the penetrating member by the controllable driver, which is controlled by the processor to maintain a withdrawal velocity of no more than about 0.006 to about 0.01 meters/second. The same cycle is illustrated in the velocity versus time plot where the penetrating member is accelerated from the start point to point A. The penetrating member coasts from A to B where the penetrating member tip contacts tissue 233. The penetrating member tip then penetrates the tissue and slows with braking force eventually applied as the maximum penetration depth is approached. The penetrating member is stopped and settling between C and D. At D, the withdrawal phase begins and the penetrating member is slowly withdrawn until it returns to the initialization point shown by E. Note that retrograde recoil from elastic and inelastic tenting was not shown in the lancing profiles for purpose of illustration and clarity.

In another embodiment, the withdrawal phase may use a dual speed profile, with the slow 0.006 to 0.01 meter per second speed used until the penetrating member is withdrawn past the contact point with the tissue, then a faster speed of 0.01 to 1 meters per second may be used to shorten the complete cycle.

Referring now to FIG. 110, yet another aspect of the present invention will now be described. To bring a new, unused penetrating member to use, the cartridge 1500 may be rotated as indicated by arrow 1502. A linear slider 1510 moves forward and backward as indicated by arrow 1512. The forward motion of the slider 1510 rotates the cartridge, among other things. In some embodiments, backward motion may be used to rotate the cartridge (it all depends on where the slider starts). Rotation occurs when a keyed gear (not shown) that the opening 1514 fits over is rotated by motion of the slider 1510. Of course, the slider 1510 in the present embodiment also actuates a plurality of other motions such as clearing the gripper, shield, and drive assembly, to lift them clear so that the cartridge 1500 can rotate.

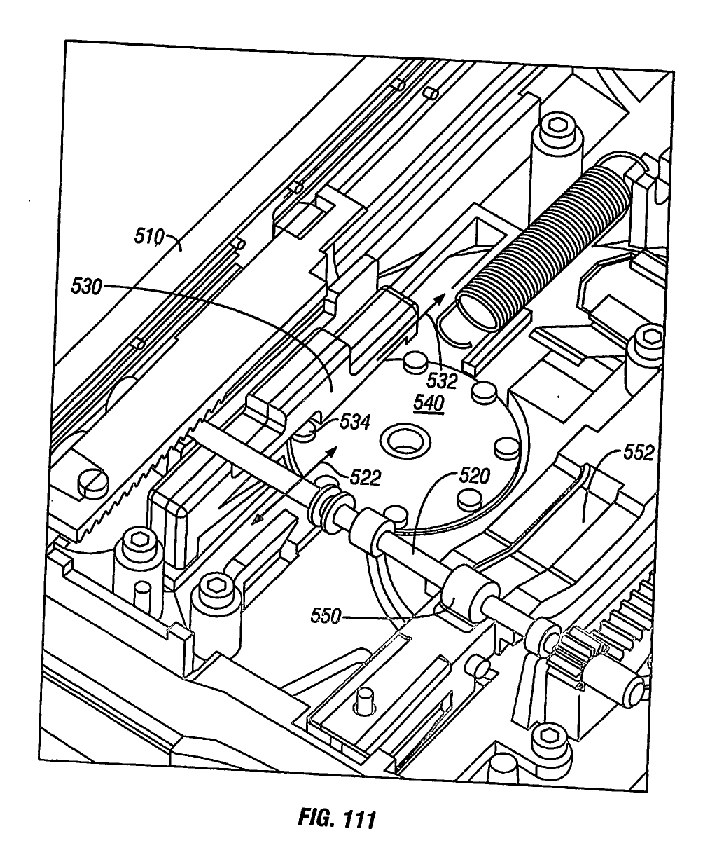

Referring now to FIG. 111 shows how movement of the slider 1510 moves rod 1520 as indicate by arrows 1522. For ease of illustration, certain portions of the device are removed to allow easier visualization of the moving parts. The motion of rod 1522 causes a second slider 1530 to move as indicated by arrow 1532 and engage a stub 1534 on the rotating wheel 1540. This wheel 1540 turns the gear the fits inside the opening 1514, which rotates the cartridge. In the present embodiment, a roller 1550 also travels on a cam surface 1552. Some of these elements are more clearly illustrated in FIGS. 7 through 9.

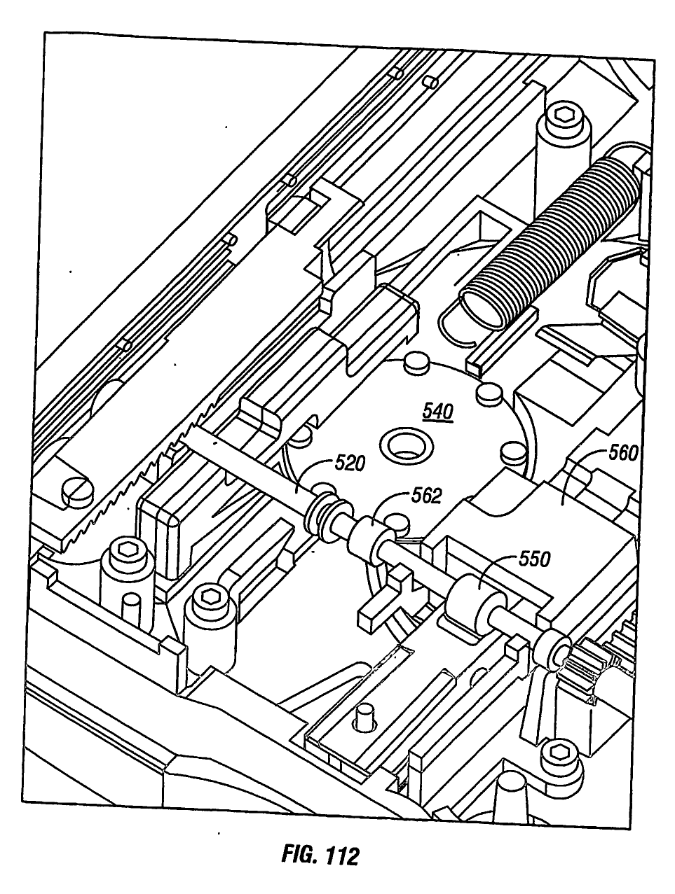

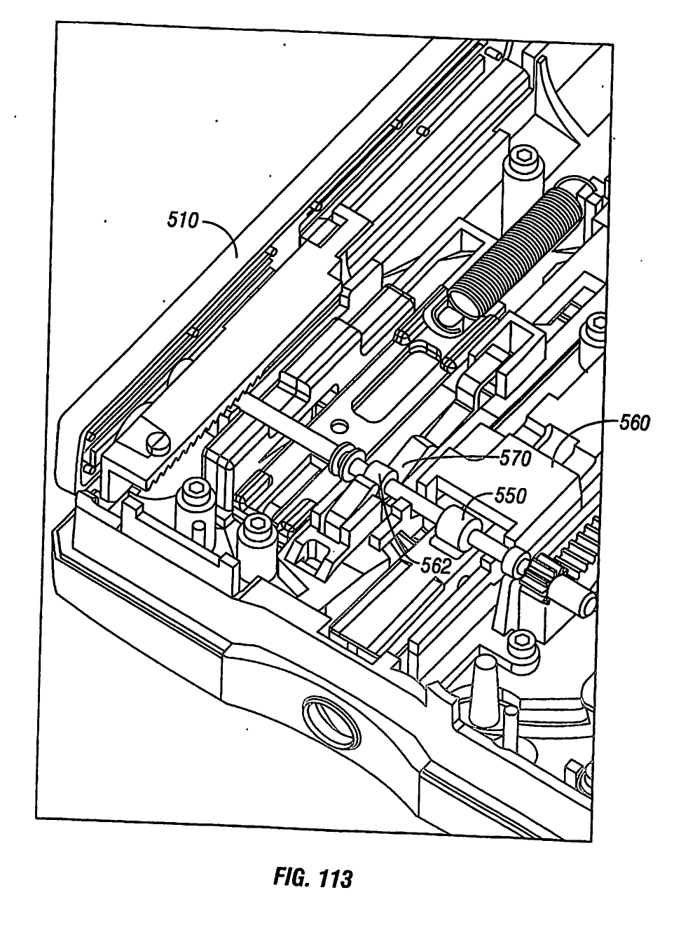

As seen in FIG. 112, the roller 1550 also move a slider 1560. The rod 1520 also includes yet another roller 1562. This roller as seen in FIG. 113, follows another cam surface 1570. The cam surfaces 1552 (FIG. 111) and 1570 (FIG. 113) allow for raising and lowering of the punch, shield, gripper, drive assembly, etc. . . . to allow for the cartridge to rotate and a new penetrating member cavity to be opened and a member loaded for firing. In some embodiments, the various steps that need to happen are similar to those described in commonly assigned copending U.S. patent application Ser. No. 10/323,623 (Attorney Docket No. 38187-2607) filed Dec. 18, 2002.

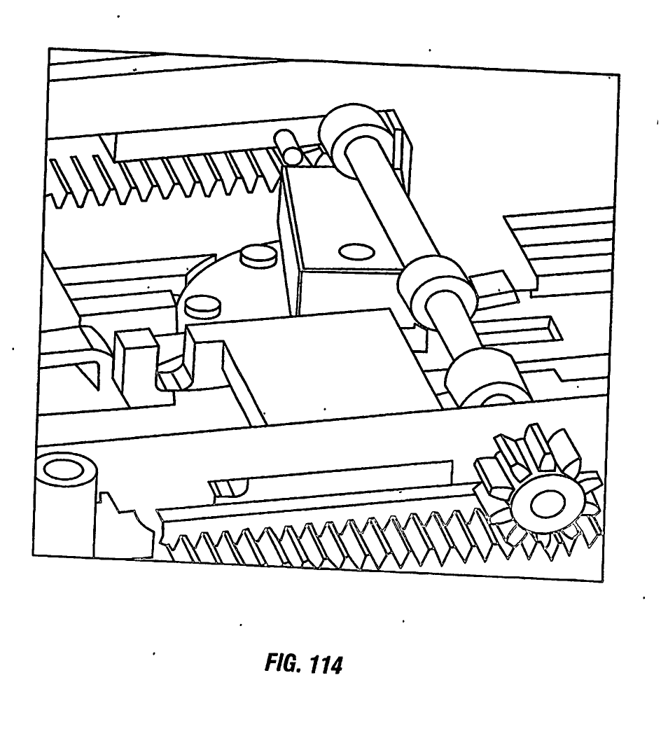

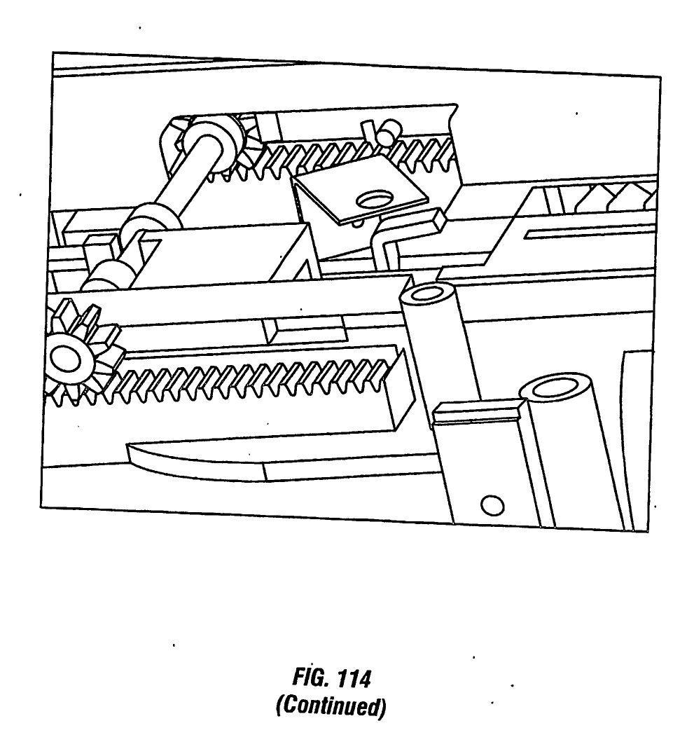

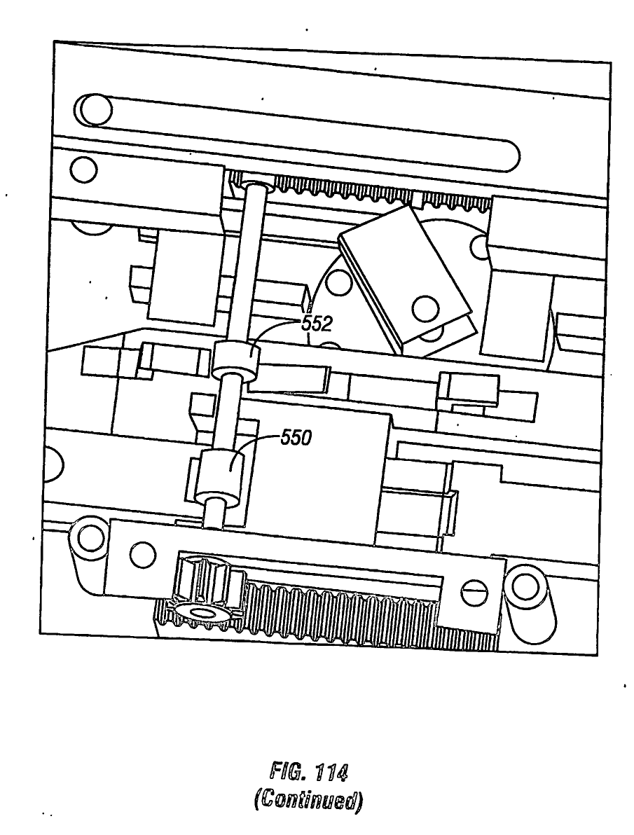

FIG. 114 shows still further embodiments of the present invention. It more clearly shows some of the elements such as roller 1562. Embodiments using the linear motion of the slider 1510 and linear motion of the rod 1520 pushing linear sliders and pushing rollers to follow linear cam surfaces are very robust and will not easily fail. In some embodiments, the sequence involves raising the gripper of the penetrating member driver to disengage the penetrating member, raise it out of the way to allow the cartridge to rotate. Simultaneously or before rotation, a punch will open the next cavity to be used for a lancing event. With gripper out of the way, the cartridge is rotated and the gripper and newly opened cavity are aligned. The gripper is then lowered to engage the new penetrating member in the cartridge cavity. Some embodiments may just lower and/raise the gripper. Other embodiments raise and lower the gripper and the entire penetrating member driver.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, with any of the above embodiments, the location of the penetrating member drive device may be varied, relative to the penetrating members or the cartridge. With any of the above embodiments, the penetrating member tips may be uncovered during actuation (i.e. penetrating members do not pierce the penetrating member enclosure or protective foil during launch). With any of the above embodiments, the penetrating members may be a bare penetrating member during launch. With any of the above embodiments, the penetrating members may be bare penetrating members prior to launch as this may allow for significantly tighter densities of penetrating members. In some embodiments, the penetrating members may be bent, curved, textured, shaped, or otherwise treated at a proximal end or area to facilitate handling by an actuator. The penetrating member may be configured to have a notch or groove to facilitate coupling to a gripper. The notch or groove may be formed along an elongate portion of the penetrating member. With any of the above embodiments, the cavity may be on the bottom or the top of the cartridge, with the gripper on the other side. In some embodiments, analyte detecting members may be printed on the top, bottom, or side of the cavities. The front end of the cartridge may be in contact with a user during lancing. The same driver may be used for advancing and retraction of the penetrating member. The penetrating member may have a diameters and length suitable for obtaining the blood volumes described herein. The penetrating member driver may also be in substantially the same plane as the cartridge. The driver may use a through hole or other opening to engage a proximal end of a penetrating member to actuate the penetrating member along a path into and out of the tissue. The present penetrating member may be used with multiple penetrating member cartridges or single penetrating member cartridges. They may be used with penetrating member cartridges which are oval, square, rectangular, triangular, hexagonal, polygonal, or other shaped or combinations of shapes. The penetrating members may be used in a bandolier configuration or held in a tape containing a plurality of penetrating members between two tapes. The penetrating members may be used electric drive devices or conventional spring-based launchers.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, with any of the above embodiments, the location of the penetrating member drive device may be varied, relative to the penetrating members or the cartridge. With any of the above embodiments, the penetrating member tips may be uncovered during actuation (i.e. penetrating members do not pierce the penetrating member enclosure or protective foil during launch). With any of the above embodiments, the penetrating members may be a bare penetrating member during launch. With any of the above embodiments, the penetrating members may be bare penetrating members prior to launch as this may allow for significantly tighter densities of penetrating members. In some embodiments, the penetrating members may be bent, curved, textured, shaped, or otherwise treated at a proximal end or area to facilitate handling by an actuator. The penetrating member may be configured to have a notch or groove to facilitate coupling to a gripper. The notch or groove may be formed along an elongate portion of the penetrating member. With any of the above embodiments, the cavity may be on the bottom or the top of the cartridge, with the gripper on the other side. In some embodiments, analyte detecting members may be printed on the top, bottom, or side of the cavities. The front end of the cartridge may be in contact with a user during lancing. The same driver may be used for advancing and retraction of the penetrating member. The penetrating member may have a diameters and length suitable for obtaining the blood volumes described herein. The penetrating member driver may also be in substantially the same plane as the cartridge. The driver may use a through hole or other opening to engage a proximal end of a penetrating member to actuate the penetrating member along a path into and out of the tissue.

Any of the features described in this application or any reference disclosed herein may be adapted for use with any embodiment of the present invention. For example, the devices of the present invention may also be combined for use with injection penetrating members or needles as described in commonly assigned, copending U.S. patent application Ser. No. 10/127,395 (Attorney Docket No. 38187-2551) filed Apr. 19, 2002. An analyte detecting member to detect the presence of foil may also be included in the lancing apparatus. For example, if a cavity has been used before, the foil or sterility barrier will be punched. The analyte detecting member can detect if the cavity is fresh or not based on the status of the barrier. It should be understood that in optional embodiments, the sterility barrier may be designed to pierce a sterility barrier of thickness that does not dull a tip of the penetrating member. The lancing apparatus may also use improved drive mechanisms. For example, a solenoid force generator may be improved to try to increase the amount of force the solenoid can generate for a given current. A solenoid for use with the present invention may have five coils and in the present embodiment the slug is roughly the size of two coils. One change is to increase the thickness of the outer metal shell or windings surround the coils. By increasing the thickness, the flux will also be increased. The slug may be split; two smaller slugs may also be used and offset by ½ of a coil pitch. This allows more slugs to be approaching a coil where it could be accelerated. This creates more events where a slug is approaching a coil, creating a more efficient system.

In another optional alternative embodiment, a gripper in the inner end of the protective cavity may hold the penetrating member during shipment and after use, eliminating the feature of using the foil, protective end, or other part to retain the used penetrating member. Some other advantages of the disclosed embodiments and features of additional embodiments include: same mechanism for transferring the used penetrating members to a storage area; a high number of penetrating members such as 25, 50, 75, 100, 500, or more penetrating members may be put on a disk or cartridge; molded body about a lancet becomes unnecessary; manufacturing of multiple penetrating member devices is simplified through the use of cartridges; handling is possible of bare rods metal wires, without any additional structural features, to actuate them into tissue; maintaining extreme (better than 50 micron—lateral—and better than 20 micron vertical) precision in guiding; and storage system for new and used penetrating members, with individual cavities/slots is provided. The housing of the lancing device may also be sized to be ergonomically pleasing. In one embodiment, the device has a width of about 56 mm, a length of about 105 mm and a thickness of about 15 mm. Additionally, some embodiments of the present invention may be used with non-electrical force generators or drive mechanism. For example, the punch device and methods for releasing the penetrating members from sterile enclosures could be adapted for use with spring based launchers. The gripper using a frictional coupling may also be adapted for use with other drive technologies.

Still further optional features may be included with the present invention. For example, with any of the above embodiments, the location of the penetrating member drive device may be varied, relative to the penetrating members or the cartridge. With any of the above embodiments, the penetrating member tips may be uncovered during actuation (i.e. penetrating members do not pierce the penetrating member enclosure or protective foil during launch). The penetrating members may be a bare penetrating member during launch. In some embodiments, the penetrating member may be a patent needle. The same driver may be used for advancing and retraction of the penetrating member. Different analyte detecting members detecting different ranges of glucose concentration, different analytes, or the like may be combined for use with each penetrating member. Non-potentiometric measurement techniques may also be used for analyte detection. For example, direct electron transfer of glucose oxidase molecules adsorbed onto carbon nanotube powder microelectrode may be used to measure glucose levels. In some embodiments, the analyte detecting members may formed to flush with the cartridge so that a “well” is not formed. In some other embodiments, the analyte detecting members may formed to be substantially flush (within 200 microns or 100 microns) with the cartridge surfaces. In all methods, nanoscopic wire growth can be carried out via chemical vapor deposition (CVD). In all of the embodiments of the invention, preferred nanoscopic wires may be nanotubes. Any method useful for depositing a glucose oxidase or other analyte detection material on a nanowire or nanotube may be used with the present invention. Additionally, for some embodiments, any of the cartridge shown above may be configured without any of the penetrating members, so that the cartridge is simply an analyte detecting device. Still further, the indexing of the cartridge may be such that adjacent cavities may not necessarily be used serially or sequentially. As a nonlimiting example, every second cavity may be used sequentially, which means that the cartridge will go through two rotations before every or substantially all of the cavities are used. As another nonlimiting example, a cavity that is 3 cavities away, 4 cavities away, or N cavities away may be the next one used. This may allow for greater separation between cavities containing penetrating members that were just used and a fresh penetrating member to be used next. For any of the embodiments herein, they may be configured to provide the various velocity profiles described.

This application cross-references commonly assigned copending U.S. patent application Ser. No. 10/323,622 (Attorney Docket No. 38187-2606) filed Dec. 18, 2002; commonly assigned copending U.S. patent application Ser. No. 10/323,623 (Attorney Docket No. 38187-2607) filed Dec. 18, 2002; and commonly assigned copending U.S. patent application Ser. No. 10/323,624 (Attorney Docket No. 38187-2608) filed Dec. 18, 2002. This application is also related to commonly assigned copending U.S. patent application Ser. Nos. 10/335,142, 10/335,215, 10/335,258, 10/335,099, 10/335,219, 10/335,052, 10/335,073, 10/335,220, 10/335,252, 10/335,218, 10/335,211, 10/335,257, 10/335,217, 10/335,212, and 10/335,241, 10/335,183, (Attorney Docket Nos. 38187-2633 through 38187-2652) filed Dec. 31, 2002. This application is also a continuation-in-part of commonly assigned, copending U.S. patent application Ser. No. 10/425,815 (Attorney Docket No. 38187-2663) filed May 30, 2003. This application is a continuation-in-part of commonly assigned, copending U.S. patent application Ser. No. 10/323,622 (Attorney Docket No. 38187-2606) filed on Dec. 18, 2002, which is a continuation-in-part of commonly assigned, copending U.S. patent application Ser. No. 10/127,395 (Attorney Docket No. 38187-2551), filed Apr. 19, 2002. This application is also a continuation-in-part of commonly assigned, copending U.S. patent application Ser. No. 10/237,261 (Attorney Docket No. 38187-2595) filed Sep. 5, 2002. This application is further a continuation-in-part of commonly assigned, copending U.S. patent application Ser. No. 10/420,535 (Attorney Docket No. 38187-2664) filed Apr. 21, 2003. This application is further a continuation-in-part of commonly assigned, copending U.S. patent application Ser. No. 10/335,142 (Attorney Docket No. 38187-2633) filed Dec. 31, 2002. This application is further a continuation-in-part of commonly assigned, copending U.S. patent application Ser. No. 10/423,851 (Attorney Docket No. 38187-2657) filed Apr. 24, 2003. This application also claims the benefit of priority from commonly assigned, copending U.S. Provisional Patent Application Ser. No. 60/422,988 (Attorney Docket No. 38187-2601) filed Nov. 1, 2002; commonly assigned, copending U.S. Provisional Patent Application Ser. No. 60/424,429 (Attorney Docket No. 38187-2602) filed Nov. 6, 2002; and commonly assigned, copending U.S. Provisional Patent Application Ser. No. 60/424,429 (Attorney Docket No. 38187-2604) filed Nov. 20, 2002. All applications listed above are incorporated herein by reference for all purposes. Additionally, U.S. Applications Ser. Nos. 60/478,041, 60/478,692, 60/478,662, and 60/483,324 are fully incorporated herein by reference for all purposes.

The publications discussed or cited herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. All publications mentioned herein are incorporated herein by reference to disclose and describe the structures and/or methods in connection with which the publications are cited.

Expected variations or differences in the results are contemplated in accordance with the objects and practices of the present invention. It is intended, therefore, that the invention be defined by the scope of the claims which follow and that such claims be interpreted as broadly as is reasonable.

1-6. (canceled)

7. A device for body fluid sampling usable with a cartridge housing a plurality of penetrating members, the device comprising:
a housing;
a penetrating member driver coupled to said housing and for use with said cartridge;
a processor for controlling said penetrating member driver to move at least one of said penetrating members at velocities which conform with a selectable velocity profile.

8. The device of claim 7 comprising a cartridge housing a plurality of penetrating members and a window allowing a user to see the cartridge while the cartridge is in said housing.

9. The device of claim 7 comprising display showing device status.

10. The device of claim 7 comprising display showing lancing performance.

11. The device of claim 7 comprising display showing lancing parameters.

12. The device of claim 7 comprising a single button for actuating said penetrating member driver along an inbound path into tissue and then an outbound path out of the tissue.

13. The device of claim 7 wherein said penetrating member driver moves an active one of said penetrating members along a velocity profile that reduces initial pain and residual pain to levels below that of known devices.

14. The device of claim 7 wherein said penetrating member driver moves an active one of said penetrating members along a velocity profile that reduces initial pain and residual pain to levels at least 1.5 times less than that of known devices.

15. The device of claim 7 wherein said penetrating member driver moves an active one of said penetrating members along a velocity profile that reduces residual pain to levels at least 2 times less than that of known devices.

16-31. (canceled)

32. A body fluid sampling device for use with a cartridge containing a plurality of penetrating members comprising:
a penetrating member driver for moving an active one of said penetrating members from a first position outward to penetrate tissue;
a display that has a screen saver.

33. (canceled)

34. A fluid sampling device comprising:
a housing;
a cartridge defining a plurality of cavities, said cartridge sized to fit within said housing; and
a plurality of penetrating members at least partially contained in said cavities of the cartridge wherein the penetrating members are slidably movable to extend outward from said cartridge to penetrate tissue, said cavities each having a longitudinal opening providing access to an elongate portion of the penetrating member;
a sterility barrier coupled to said cartridge, said sterility barrier covering a plurality of the longitudinal openings, wherein the sterility barrier covering the lateral openings is configured to be moved so that the elongate portion may be accessed by the gripper without touching the barrier; and
a slider located on a surface of said housing, said slider movable in a linear direction to rotate said cartridge to bring an unused penetrating member into position for use;
a tooth gear coupled to said slider to control a distance said slider can travel;
a follower coupled to said slider;
a cam surface engaged by said follower to lift said cartridge a desired distance above a first position to allow for rotation of the cartridge without engaging a gripper used to advance the penetrating member.

35. The device of claim 34 wherein said cam surface is aligned parallel to said slider.

36. The device of claim 34 wherein said linear motion of the cam rotates the cartridge and moves a plunger to break the sterility barrier on the cartridge.

37. The device of claim 34 wherein cam surface comprise a linear strip of material with at least two raised portions and two depressed portions.

38-40. (canceled)

41. A device comprising:
a housing;
a penetrating member driver;
a cartridge containing a plurality of penetrating members;
a display on said cartridge;
a linear slider on the housing, said slider coupled to a rod;
said rod moving with said slider, said rod having at least one roller
using the linear motion of the slider to rotate the cartridge, punch open a new cavity and load a new penetrating member.

42. A method of indexing comprising:
moving a linear slider;
said linear slider coupled to a rod;
said rod moving with said slider, said rod having at least one roller
using the linear motion of the slider and linear motion of the rod to push at least one linear slider and to roll a roller along a linear cam surfaces to lift clear a drive assembly, rotate the cartridge, punch open a new cavity and load a new penetrating member.
